(19) United States
(12) Patent Application Publication
(10) Pub. No.: US 2025/0223330 A1
(43) Pub. Date: Jul. 10, 2025

(54) DESIGNED CYTOKINE COMPOSITIONS AND METHODS OF USE

(71) Applicant: Outpace Bio, Inc., Seattle, WA (US)

(72) Inventors: Scott BOYKEN, Seattle, WA (US); Marc LAJOIE, Seattle, WA (US); Thaddeus M. DAVENPORT, Seattle, WA (US); Howell MOFFETT, Seattle, WA (US); Paul Joseph SAMPLE, Seattle, WA (US); Brian WEITZNER, Colorado Springs, CO (US); Andrew Howen NG, San Francisco, CA (US)

(21) Appl. No.: 18/727,646

(22) PCT Filed: Jan. 11, 2023
(86) PCT No.: PCT/US2023/060508

§ 371 Date: Jul. 9, 2024

Related U.S. Application Data
(60) Provisional application No. 63/298,623, filed on Jan. 11, 2022.
(60) Provisional application No. 63/479,178, filed on Jan. 9, 2023.
(60) Provisional application No. 63/479,177, filed on Jan. 9, 2023.
(60) Provisional application No. 63/479,176, filed on Jan. 9, 2023.

(51) Int. Cl. C07K 14/55; C07K 14/54

(52) U.S. Cl. CPC C07K 14/55; C07K 14/5443; C07K 2319/30

(57) ABSTRACT

The disclosure provides a designed cytokine comprising alpha helices H1, H2, H3, and H4, wherein, from an amino terminus to a carboxy terminus, a first loop (L1) connects H1 and H4; a second loop (L2) connects H4 and H2; a third loop (L3) connects H2 and H3; and wherein the polypeptide binds to IL-2 receptor βγ (IL-2Rβγ).

### CROSS-REFERENCE TO RELATED APPLICATIONS

This application claims the benefit of U.S. Provisional Application No. 63/298,623, filed Jan. 11, 2022; U.S. Provisional Application No. 63/479,176, filed Jan. 9, 2023; U.S. Provisional Application No. 63/479,177, filed Jan. 9, 2023; U.S. Provisional Application No. 63/479,178, filed Jan. 9, 2023; all of which are incorporated herein in their entirety.

### FIELD OF THE DISCLOSURE

The disclosure relates to the fields of immunology, gene therapy, protein design, cell signaling, biologics and cellular therapies.

### BACKGROUND

IL-2 has shown promise as an anti-cancer immunotherapy, but efficacy and safety have been diminished by dose-limiting toxicity due to preferential stimulation of Treg cells and dose-limiting toxicity due to IL-2Rα binding. The disclosure provides a non-naturally occurring designed cytokine as a solution to this unmet need in the art.

### SUMMARY

The disclosure provides non-naturally occurring designed cytokines having enhanced function and improved stability when compared to wild type IL-2. Non-naturally occurring designed cytokines of the disclosure, also referred to herein as IL-2/15 cytokines named for their dual functionality of signaling through both the IL-2 and IL-15 receptors, reduce preferential Treg stimulation, potentiates T cell subtype targeting, stabilizes protein folding, and reduces immunogenicity. In some embodiments, designed cytokines eliminate preferential Treg stimulation. In some embodiments, designed cytokines eliminate immunogenicity. In some embodiments, designed cytokines provide enhanced function and improved stability with reduced post-translational modifications compared to the post-translational modifications made to wild type IL-2. In some embodiments, designed cytokines provide enhanced function and improved stability without post-translational modifications.

The disclosure provides a Designed Cytokine comprising alpha helices H1, H2, H3, and H4, wherein: from an amino terminus to a carboxy terminus, a first loop (L1) connects H1 and H4; a second loop (L2) connects H4 and H2; a third loop (L3) connects H2 and H3; and wherein the polypeptide binds to IL-2 receptor By (IL-2Rβγ), also referred to as IL-2/15Rβγ. In some embodiments, the designed cytokine does not bind to IL-2 receptor alpha (IL-2Rα).

In some embodiments of the Designed Cytokines of the disclosure, the Designed Cytokine comprises one or more of a sequence of SEQ ID NO: 1-350.

In some embodiments of the Designed Cytokine of the disclosure, the polypeptide comprises one or more of: (a) a sequence of SEQ ID NO: 1-350 and (b) a sequence having at least 70% identity to a sequence of (a).

In some embodiments of the Designed Cytokine of the disclosure, the polypeptide comprises a sequence of SEQ ID NO: 1-38 or 150-350.

In some embodiments of the Designed Cytokine of the disclosure, the L3 loop comprises a sequence of QSKNFHLR (SEQ ID NO: 132).

In some embodiments of the Designed Cytokine of the disclosure, the H1 helix comprises a sequence of SEQ ID NO: 39-51.

In some embodiments of the Designed Cytokine of the disclosure, the H2 helix comprises a sequence of SEQ ID NO: 52-77.

In some embodiments of the Designed Cytokine of the disclosure, the H3 helix comprises a sequence of SEQ ID NO: 78-97.

In some embodiments of the Designed Cytokine of the disclosure, the H4 helix comprises a sequence of SEQ ID NO: 98-101.

In some embodiments of the Designed Cytokine of the disclosure, the L1 loop comprises a sequence of SEQ ID NO: 102-111.

In some embodiments of the Designed Cytokine of the disclosure, the L2 loop comprises a sequence of SEQ ID NO: 112-131.

In some embodiments of the Designed Cytokine of the disclosure, the polypeptide comprises a sequence of SEQ ID NO: 1-38 or 150-350.

In some embodiments of the Designed Cytokine of the disclosure, the polypeptide comprises the sequence of

APTSSSTKKTQLQLEHLLLDLQMILNGINNMNADPELVEFLNRWITFCQ
SIISTGSLEDLKHLQALEEELKPLEEVLNLAQSKNFHLNPRDLISNINV
LVLELK.

In some embodiments of the Designed Cytokine of the disclosure, the polypeptide comprises the sequence of

APTSSSTKKTQLQLEHLLLDLQMILNGINNMNADPELVEFLNRWITFCQ
SIISTGSLEDLKHLQALEEELKPLEEVLNLAQSKNFHLRPRDLISNINV
IVLELK.

In some embodiments of the Designed Cytokine of the disclosure, the polypeptide comprises the sequence of

APTSSSTKKTQLQLEHLLLDLQMILNGINNMNADPELVEFLNRWITFCQ
SIISTGSLEDLKHLQALEEELKPLEEVLNLAQSKNFHLRPRDLISNINV
IVLELK

In some embodiments of the Designed Cytokine of the disclosure, the polypeptide comprises the sequence of

APTSSSTKKTQLQLEHLLLDLQMILNGINNMNADPELVEFLNRWITFCQ
SIISTLTAGGSLSGDLKHLQNLSEELKPLEEVLNLAQSKNFHLRPRDLI
SNINVIVLELK.

In some embodiments of the Designed Cytokine of the disclosure, the polypeptide comprises the sequence of

APTSSSTKKTQLQLEHLLLDLQMILNGINNMNADPELVEFLNRWITFCQ
SIISTGSVDPEELAKELQKLEEELKPLEEVLNLAQSKNFHLRPRDLISN
INVIVLELK.

In some embodiments of the Designed Cytokine of the disclosure, the polypeptide is operably linked to a targeting moiety. In some embodiments, the polypeptide comprises a targeting moiety. In some embodiments, a fusion protein comprises the polypeptide and a targeting moiety. In some embodiments, the targeting moiety binds to a component of a tumor microenvironment (TME). In some embodiments, the targeting moiety binds to one or more of T-cell surface glycoprotein CD8 (also known as cluster of differentiation 8), Programmed cell death protein 1 (PD-1), Programmed death-ligand 1 (PD-L1; also known as cluster of differentiation 274 (CD274) or B7 homolog 1 (B7-H1) polypeptide), T-cell immunoreceptor with Ig and ITIM domains (TIGIT), Cytotoxic T-lymphocyte protein 4 (CTLA4), Lymphocyte activation gene 3 protein (LAG3), T-cell immunoglobulin mucin receptor 3 (TIM3). In some embodiments, the targeting moiety binds CD8. In some embodiments, the targeting moiety binds PD-1. In some embodiments, the targeting moiety binds PD-L1. In some embodiments, the targeting moiety comprises an antibody, an antibody mimetic, or a functional fragment thereof. In some embodiments, the targeting moiety comprises one or more of a monoclonal antibody, an antigen-binding fraction (Fab), a single-chain variable fraction (scFv), a domain antibody, one or more of a heavy chain (VH) and a light chain (VL) domain of an immunoglobulin (Ig) polypeptide or gene encoding the same, a heavy-chain antibody (a VH or a VHH), a camelid or camelid-like structured antibody, and a nanobody. In some embodiments, the targeting moiety comprises a scFv. In some embodiments, the targeting moiety comprises a VHH.

In some embodiments of the Designed Cytokine of the disclosure, the polypeptide is operably linked to a tether. In some embodiments, the polypeptide comprises a tether. In some embodiments, a fusion protein comprises the polypeptide and a tether. In some embodiments, the tether comprises one or more of a nucleic acid sequence, an amino acid sequence, a small molecule. In some embodiments, the tether comprises a sequence isolated or derived from a transmembrane sequence. In some embodiments, the tether comprises the sequence

PLFIPVAVMVTAFSGLAFIIWLARRLKKGKK.

In some embodiments of the Designed Cytokine of the disclosure, the polypeptide is operably linked to a second cytokine or a second Designed Cytokine. In some embodiments, the polypeptide comprises a second cytokine or a second Designed Cytokine. In some embodiments, a fusion protein comprises the polypeptide and a second cytokine or a second Designed Cytokine. I

In some embodiments of the Designed Cytokine of the disclosure, the second cytokine comprises a sequence isolated or derived from one or more of an IL-2 polypeptide, an IL-12 polypeptide, an IL-15 polypeptide, an IL-18 polypeptide, an IL-21 polypeptide, an IL-23 polypeptide, an interferon alpha polypeptide, an interferon beta polypeptide, an interferon gamma polypeptide, and an interferon omega polypeptide. In some embodiments, the polypeptide comprises a first targeting moiety and the second cytokine comprises a second targeting moiety. In some embodiments, the first targeting moiety and the second targeting moiety are identical. In some embodiments, the first targeting moiety and the second targeting moiety are not identical.

In some embodiments of the Designed Cytokine of the disclosure, the second Designed Cytokine comprises a sequence of any one or more of SEQ ID NO: 1-38 or 150-350. In some embodiments, the polypeptide comprises a first targeting moiety and the second Designed Cytokine comprises a second targeting moiety. In some embodiments, the first targeting moiety and the second targeting moiety are identical. In some embodiments, the first targeting moiety and the second targeting moiety are not identical.

In some embodiments of the Designed Cytokine of the disclosure, the polypeptide comprises a first tether and the second cytokine comprises a second tether. In some embodiments, the first tether and the second tether are identical. In some embodiments, the first tether and the second tether are not identical.

In some embodiments of the Designed Cytokine of the disclosure, the polypeptide comprises a first tether and the second Designed Cytokine comprises a second tether. In some embodiments, the first tether and the second tether are identical. In some embodiments, the first tether and the second tether are not identical.

The disclosure provides a nucleic acid encoding the designed cytokine of the disclosure or a fusion protein comprising the designed cytokine of the disclosure. In some embodiments, the nucleic acid further comprises a regulatory element capable of driving expression of the designed cytokine. In some embodiments, the regulatory element comprises a promoter. In some embodiments, the promoter comprises a minimal promoter. In some embodiments, the minimal promoter comprises a sequence isolated or derived from one or more of minimal promoter-1 (“minP1”), YB-TATA and human beta globin. In some embodiments, the minP1 comprises a sequence of AGAGGGTATATAAAAGCTCGACTTCCAG. In some embodiments, the minimal promoter comprises the sequence of TAGAGGGTATATAATGGGGGCCACTAGTCTACTACCAGAAAGCTTGGTACCGAGCT CGGATCCAGCCACC. In some embodiments, the minimal promoter comprises the sequence of CTAGAGGGTATATAATGGGGGCCACTAGTCTACTACCAGAAAGCTTGGTACCGAGC TCGGATCCAGCCACC. In some embodiments, the promoter is inducible. In some embodiments, the regulatory element comprises a response element. In some embodiments, the regulatory element comprises a noncoding or an untranslated sequence isolated or derived from one or more of NFAT, NFkB, REL, RELA, IRF2, GATA3 and ATF3. In some embodiments, the regulatory element comprises a noncoding or an untranslated sequence isolated or derived from a GATA3 gene. In some embodiments, the regulatory element comprises a noncoding or an untranslated sequence isolated or derived from RELA. In some embodiments, the response element comprises a repeated sequence.

The disclosure provides a vector comprising a nucleic acid of the disclosure. In some embodiments, the vector comprises an expression vector. In some embodiments, the vector comprises a delivery vector. In some embodiments, the vector further comprises a sequence encoding an exogenous receptor. In some embodiments, the exogenous receptor comprises an antigen binding moiety. In some embodiments, the exogenous receptor comprises a T Cell Receptor (TCR). In some embodiments, the exogenous receptor comprises a chimeric antigen receptor (CAR). In some embodiments, the antigen is expressed on or secreted within one or more of a tumor cell, a cancer cell, a component of a TME, and a TME.

In some embodiments of the disclosure, a vector is a non-viral vector. In some embodiments, a non-viral vector comprises one or more of a plasmid, a nucleic acid, a polymer, a micelle, a polymersome, an exosome, a lysosome, a nanoparticle, and any combination thereof.

In some embodiments of the disclosure, a vector is a viral vector. In some embodiments, the viral vector comprise a sequence isolated or derived from a sequence of a virus, a lentivirus or a lentiviral vector.

The disclosure provides a cell comprising a designed cytokine of the disclosure. The disclosure provides a cell comprising a nucleic acid of the disclosure. The disclosure provides a cell comprising a vector of the disclosure. In some embodiments of the disclosure, the cell is a mammalian cell. In some embodiments of the disclosure, the cell is a human cell. In some embodiments of the disclosure, the cell is a primary cell. In some embodiments of the disclosure, the cell is a cultured cell. In some embodiments, the cultured cell is an immortalized cell. In some embodiments, the cell is ex vivo or in vitro. In some embodiments, the cell is in vivo. In some embodiments, the cell is an immune cell. In some embodiments, the cell is a stem cell or a precursor cell capable of producing the immune cell. In some embodiments, the stem cell is a hematopoietic stem cells (HSC), an induced pluripotent stem cell (iPSC) or a dedifferentiated immune cell. In some embodiments, the immune cell is a T lymphocyte (T cell), a B lymphocyte (B cell), a macrophage or a natural killer (NK) cell. In some embodiments, the immune cell is a T cell. In some embodiments, the T cell is an alpha beta T cell. In some embodiments, the T cell is a gamma delta T cell. In some embodiments, the immune cell is a NK cell.

The disclosure provides a composition comprising a designed cytokine of the disclosure. The disclosure provides a composition comprising a nucleic acid of the disclosure. The disclosure provides a composition comprising a vector of the disclosure. The disclosure provides a composition comprising a cell of the disclosure.

The disclosure provides a pharmaceutical composition comprising one or more of (1) a designed cytokine of the disclosure, a nucleic acid of the disclosure, a vector of the disclosure, and a cell of the disclosure and (2) a pharmaceutically acceptable carrier.

The disclosure provides a use of a designed cytokine of the disclosure, a nucleic acid of the disclosure, a vector of the disclosure, a cell of the disclosure or a pharmaceutical composition of the disclosure in the manufacture of a medicament for the treatment of a disease or condition. In some embodiments, the disease or disorder comprises a cancer or a subtype thereof. In some embodiments, the cancer or the subtype thereof comprises a liquid cancer. In some embodiments, the cancer or the subtype thereof comprises a hematological cancer. In some embodiments, the cancer or the subtype thereof comprises a solid cancer.

The disclosure provides a designed cytokine of the disclosure, a nucleic acid of the disclosure, a vector of the disclosure, a cell of the disclosure or a pharmaceutical composition of the disclosure for use in the treatment of a disease or condition. In some embodiments, the disease or disorder comprises a cancer or a subtype thereof. In some embodiments, the cancer or the subtype thereof comprises a liquid cancer. In some embodiments, the cancer or the subtype thereof comprises a hematological cancer. In some embodiments, the cancer or the subtype thereof comprises a solid cancer.

The disclosure provides a method of treating a disease or disorder comprising administering to a subject an effective amount of a designed cytokine of the disclosure, a nucleic acid of the disclosure, a vector of the disclosure, a cell of the disclosure or a pharmaceutical composition of the disclosure, wherein a severity of a sign or symptom of the disease or disorder is decreased, thereby treating the disease or disorder. In some embodiments, the disease or disorder comprises a cancer or a subtype thereof. In some embodiments, the cancer or the subtype thereof comprises a liquid cancer. In some embodiments, the cancer or the subtype thereof comprises a hematological cancer. In some embodiments, the cancer or the subtype thereof comprises a solid cancer.

The disclosure provides a method of preventing a disease or a disorder, comprising administering to a subject an effective amount of a designed cytokine of the disclosure, a nucleic acid of the disclosure, a vector of the disclosure, a cell of the disclosure or a pharmaceutical composition of the disclosure, wherein an onset or a relapse of a sign or symptom of the disease or disorder is delayed or inhibited, thereby preventing the disease or disorder. In some embodiments, the disease or disorder comprises a cancer or a sub type thereof. In some embodiments, the cancer or the subtype thereof comprises a liquid cancer. In some embodiments, the cancer or the subtype thereof comprises a hematological cancer. In some embodiments, the cancer or the subtype thereof comprises a solid cancer.

### BRIEF DESCRIPTION OF THE DRAWINGS

FIG. 1A is a schematic diagram depicting a naturally occurring form of IL-2 (left) and an exemplary non-naturally occurring designed cytokine of the disclosure, in which one or more loops are redesigned to alter binding of the designed cytokine to the naturally-occurring or endogenous IL-2 receptor beta (IL-2Rβ) or IL-2 receptor gamma (IL-2Rγ, also known as the common gamma chain receptor), but not IL-2 receptor alpha (IL-2Rα). As shown in this diagram, in some embodiments, exemplary non-naturally occurring designed cytokines of the disclosure contain a sequence isolated or derived from a helix of an IL-2 cytokine with the connectivity of the designed cytokine either according to the 1-4-2-3 plan depicted herein or according to a structure of four helices connected by loops lacking the capacity to bind and/or activate the IL-2 receptor alpha by lacking an interface for binding to the IL-2 receptor alpha.

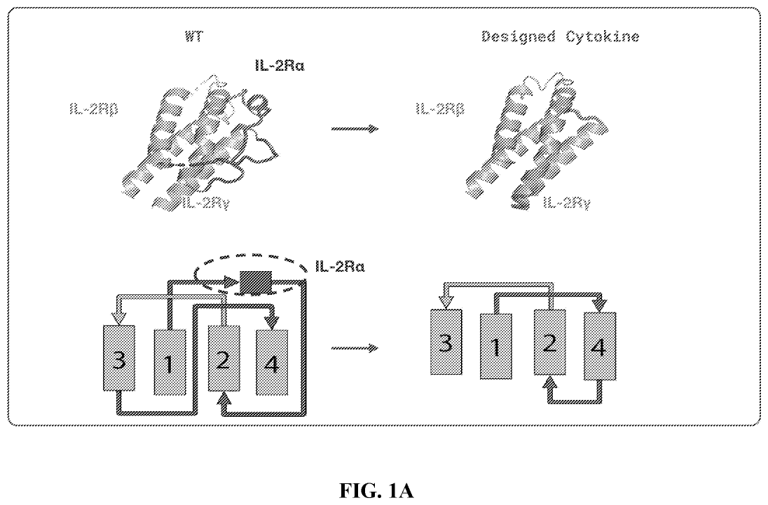

FIG. 1B is a series of schematic diagram depicting on the left half, an updated diagram highlighting the interactions with each subunit of the IL-2 receptor that allow for IL-15 functionality and on the right half, a diagram according to FIG. 1A, showing a correspondence between the abstract schematic and the ribbon schematic. As depicted in this figure, not only does the designed cytokine lack an interface for binding and/or activating the alpha subunit of the IL-2 receptor, but also, the “helix 4” of the designed cytokine binds to the gamma subunit of the IL-2 receptor, which is shared with the IL-15 Receptor, and also, “helix 1” and “helix 3” of the designed cytokine bind to IL-2 receptor beta, which is also shared with the IL-15 Receptor. As a consequence, the designed cytokines of the disclosure have functionality through the IL-2 Receptor and/or the IL-15 Receptor.

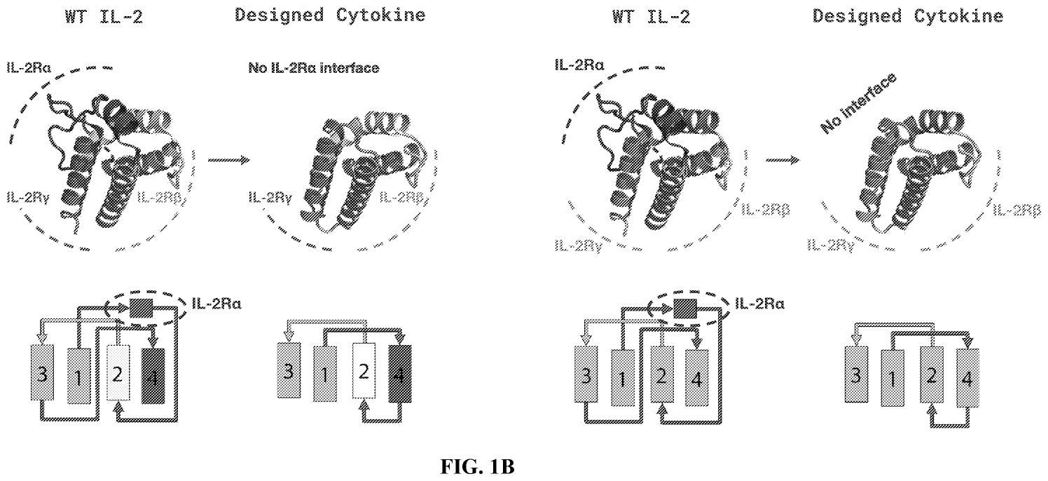

FIG. 2 is a schematic diagram depicting T cell subtype-specific IL-2 WT signaling. As shown in FIG. 1A-B, designed cytokines of the disclosure signal through one or more of the beta and gamma subunits of the IL-2 Receptor.

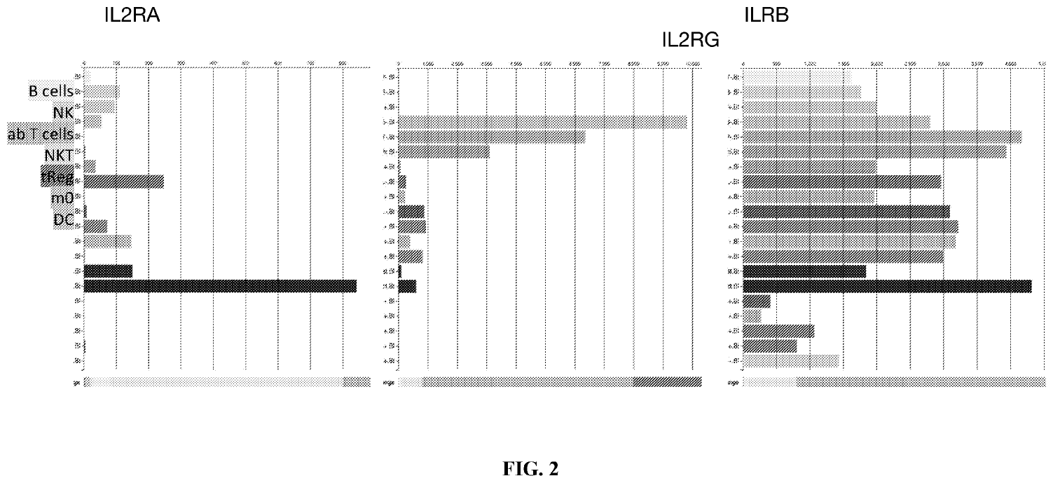

FIG. 3A is a schematic diagram showing that designed cytokines of the disclosure, as beta/gamma binders, may target specific cell subtypes. Moreover, designed cytokines of the disclosure may be linked to or may comprise a targeting moiety. While the targeting moiety may bind to any target, including, but not limited to, an antigen present or expressed within a cell or other component of a tumor microenvironment (TME) such as CD8 or PDL1.

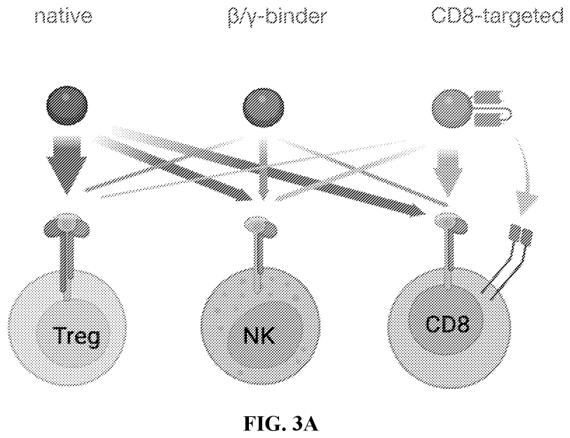

FIG. 3B is a schematic diagram showing that designed cytokines of the disclosure, may target specific cell subtypes, as a result of (1) being designed as beta/gamma binders, (2) targeted to a cellular subtype, (3) tethered to a T cell or subtype thereof (e.g., alpha-beta, gamma-delta, CD8+, CD4+, natural killer T cell (NKT cell)), or any combination thereof. Targeted designed cytokines may be linked to or comprise a targeting moiety, which may bind to any target, including, but not limited to, an antigen present or expressed within a cell or other component of a tumor microenvironment (TME) such as CD8 or PDL1. Tethered designed cytokines may be linked to or comprise a tether, which may comprise a nucleic acid, an amino acid, a small molecule or any combination thereof, and which may link the designed cytokine to cell (e.g., T-cell, NK cell or other immune cell) expressing the designed cytokine or to any component of that cell. In some embodiments, the use of a targeting moiety may be advantageous to localize the designed cytokine or a cell expressing it to a solid tumor or to a TME for localized signaling from the designed cytokine (as opposed to systemic signaling from the designed cytokine). In some embodiments, the use of a tether may be advantageous to minimize bystander activity of cells proximal to either the target cell or to the cell expressing the designed cytokine. In some embodiments, the use of a tether may be advantageous for the treatment of liquid or hematological tumors. In some embodiments, the use of a tether may be advantageous for use with T cells, wherein the bystander cell may be a Natural Killer (NK) cell.

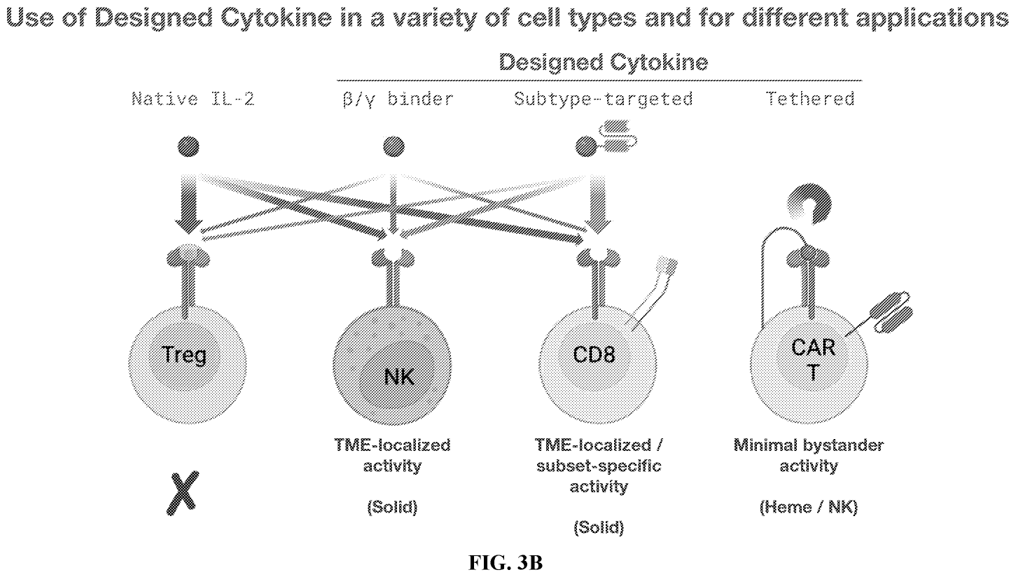

FIG. 4 is a schematic diagram showing that designed cytokines of the disclosure lack an interface to bind and/or activate the alpha subunit of the IL-2 Receptor. Moreover, the ablation of this interface retains native interfaces for binding IL-2 Receptor beta and IL-2 Receptor gamma subunits that are shared between the IL-2 and IL-15 Receptors. The design of short and structured loops between helices increases protein stability when compared to the loop length, loop structure and/or protein stability of wild type (WT) IL-2.

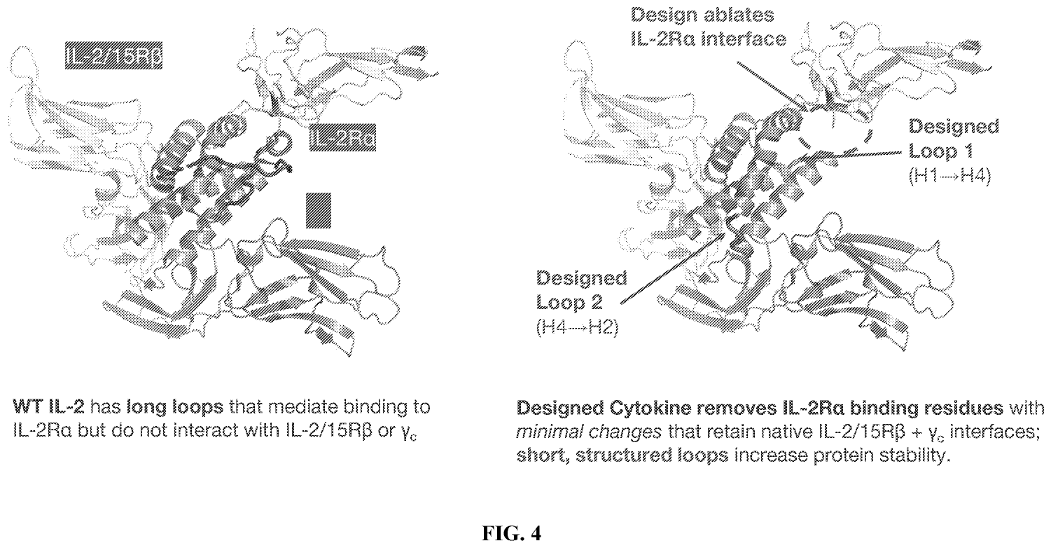

FIG. 5A is a pair of graphs demonstrating that of thirty-eight (38) designed cytokines tested alone with a WT IL-2 and a negative control, the common structural arrangement of the designed cytokines ablates IL-2 Receptor alpha biding in all designed cytokines (top) while retaining signaling through the beta and gamma subunits of the IL-2 Receptor (the gamma subunit being shared with the IL-15 Receptor).

FIG. 5B is a series graphs demonstrating that designed cytokines of the disclosure bind to IL-2/15Rβγ with very low nanomolar affinity. For each plot shown the affinity (measured in nanomolar (nm) concentration) of either WT IL2 (top) or a designed cytokine (bottom) when contacted with either IL-1Rα (left) or IL-2/15Rβγ (right) is show as a function of time (measured in seconds(s)). Compositions of either WT IL-2 or a designed cytokine were tested at between 0.9 nM and 3000 nM to generate these plots.

FIG. 6 is a series graphs demonstrating that activity of designed cytokines of the disclosure may be tuned over a wide range. As used throughout the disclosure, designed cytokines may be “tuned” to perform optimally either within a desired cell type, a desired TME, a desired target cell, and/or a desired cytokine receptor (e.g., a ratio of activity between IL-2 and IL-15). An initial design of a designed cytokine may comprise a sequence of the disclosure or a sequence having at least 70% identity thereto. An optimized design of a designed cytokine may comprise a sequence of the disclosure or a sequence having at least 70% identity thereto. An optimized design of a designed cytokine may comprise a sequence derived from a sequence of the disclosure that is generated according to the teachings provided by the disclosure. Top plot: cell proliferation was assessed by measuring the percentage of phosphorylated Signal transducer and activator of transcription 5 (STAT5) of CD8+ T cells following stimulation with antigen as a function of concentration of either WT IL-2 or designed cytokine provided (concentration measured as nanograms per milliliter (ng/ml)). Designed Cytokines of the disclosure may be “optimized” as shown in the top right plot to have a desired activity profile. With respect to the activity of binding IL-2/15Rβγ as opposed to IL-2Rα, the two bottom plots show that each Designed Cytokine binds to IL-2/15Rβγ with a distinct activity while no Designed Cytokine binds to IL-2Rα. In this figure, Design 1=Designed Cytokine No. 169, Design 2=Designed Cytokine No. 175, and Design 3=Designed Cytokine No. 153.

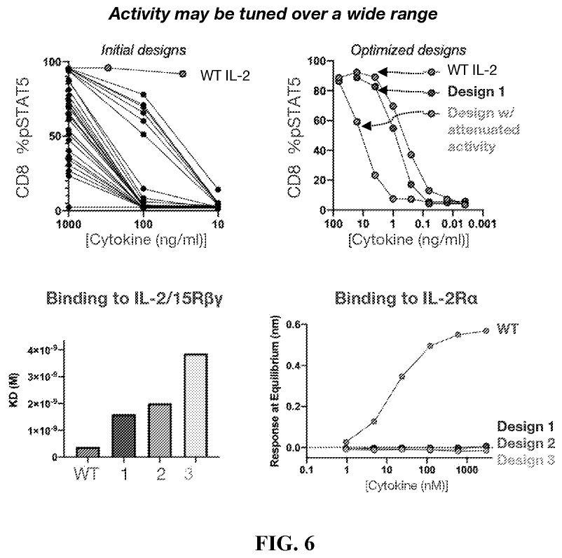

FIG. 7 is a series graphs demonstrating that Designed Cytokines do not have a regulatory T cell (Treg) preference when compared to WT IL-2, however, Designed Cytokines retain NK and CD8+ T cell activity. WT IL-2 (top plots) or Designed Cytokine (bottom plots) were contacted to CD8+ T cells, NK cells or Tregs and cell proliferation was assessed by measuring the percentage of phosphorylated Signal transducer and activator of transcription 5 (STAT5) of CD8+ T cells following stimulation with antigen as a function of concentration of either WT IL-2 or designed cytokine provided (shown as a nanomolar (nM) concentration). Two cell donors are shown (left versus right plots). Whereas the WT IL-2 favors activation of Tregs, the Designed Cytokines demonstrate a reduced Treg potency that eliminates the WT IL-2 Treg preference.

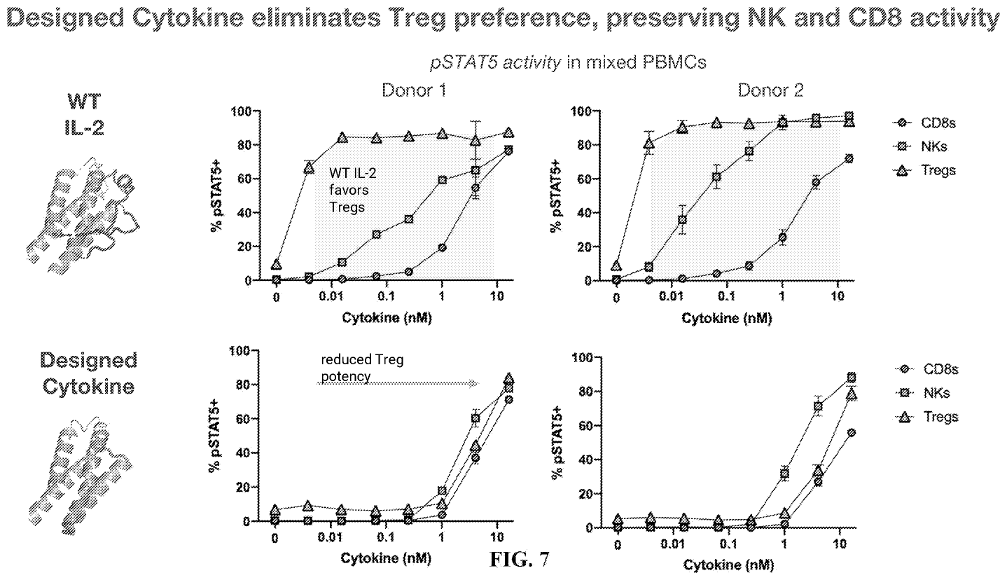

FIG. 8 is a graph demonstrating in vitro cell proliferation in the presence of 1 nanomolar (nM) WT IL-2 or Designed Cytokine. The measure on the Y-axis is a relative scale in which the cell expansion, measured as shown in FIG. 7, is normalized to WT activity (held at a value of 1.0). Within either a population of mixed peripheral blood mononuclear cells (PBMCs) or an isolated populations of NK cells or activated CD8+ T cells, respectively, Designed Cytokines eliminate the Treg preference shown by WT IL-2 while retaining the ability to proliferate NK and CD8+ T cells.

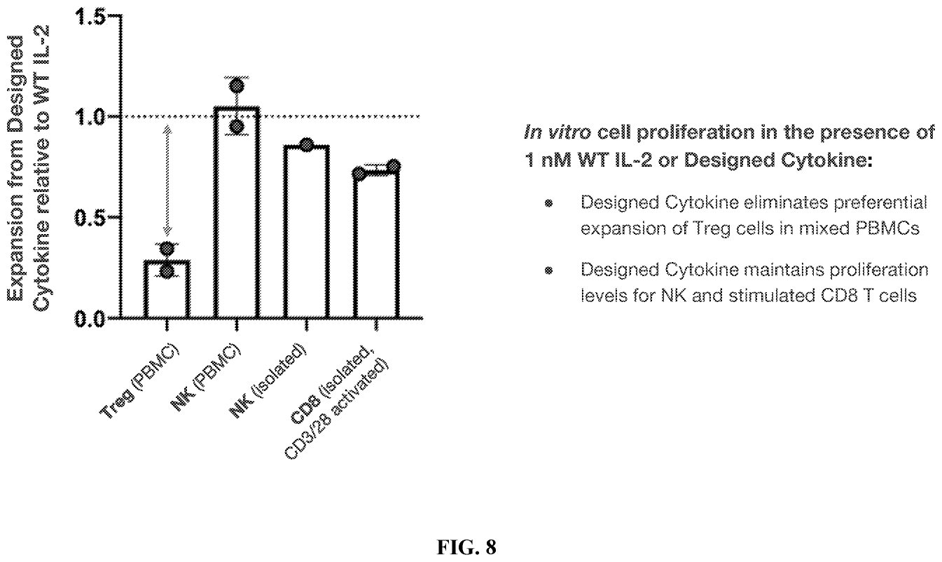

FIG. 9 is a series graphs demonstrating that Designed Cytokines do not have a regulatory T cell (Treg) preference when compared to WT IL-2, however, Designed Cytokines retain NK and CD8+ T cell activity. For each plot, either WT IL-2 (top) or Designed Cytokines (bottom) were provided in vitro to mixed PBMCs (left), isolated NK cells (middle) and CD3/28-activated T cells, in nanomolar (nM) concentrations to measure fold expansion. Of note, the bottom left plot shows a significant right shift of the activity for Treg cells as the Designed Cytokine has a reduced potency for Treg cells when compared to WT IL-2.

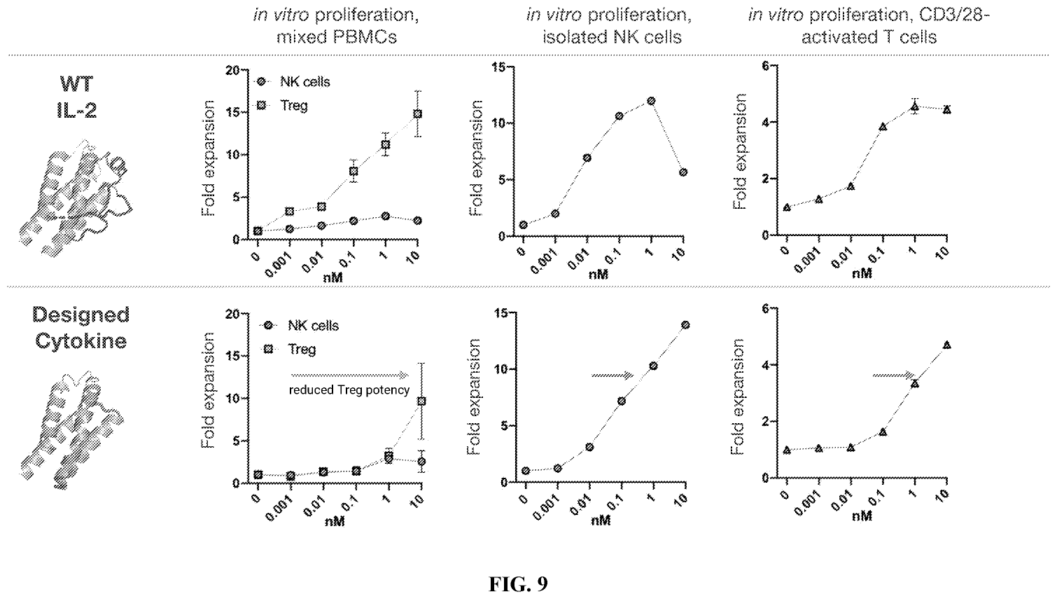

FIG. 10 is a schematic diagram and a graph demonstrating the enhanced activity of T cells expressing both a CAR and a Designed Cytokine of the disclosure. The schematic diagram depicts the experimental design that was used to generate the data displayed the graph. The fold expansion of cells mock transfected, transfected with only a chimeric antigen receptor (CAR), transfected with a CAR in combination with a WT IL-2 construct, or transfected with a CAR and a construct providing a Designed Cytokine of the disclosure were repeatedly stimulated by plate-bound antigen and, following each round, the fold expansion was assessed.

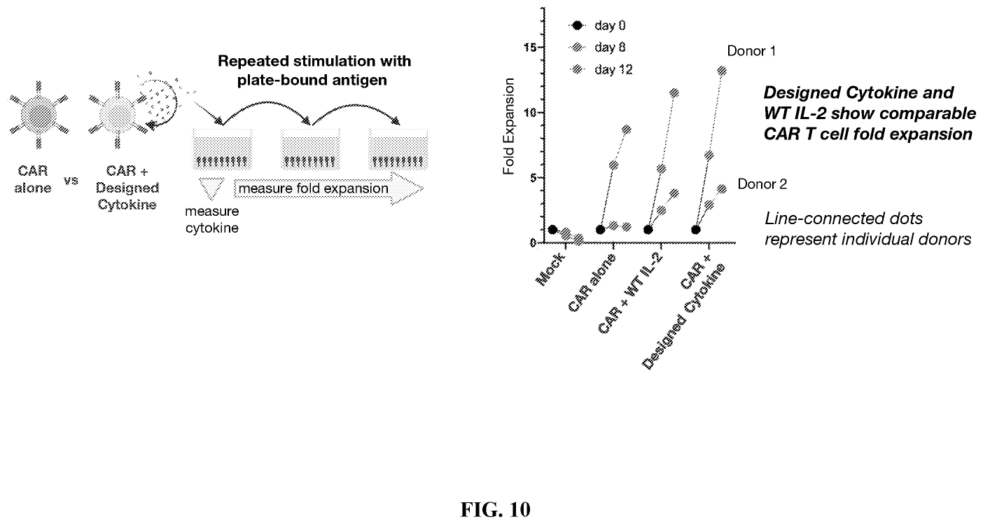

FIG. 11 is a schematic diagram and a graph demonstrating that T-cell activation induced secretion of Designed Cytokines using inducible promoters capable of driving expression, conditionally, of the Designed Cytokine in response to antigen receptor signaling (e.g., CAR or TCR) or cell state (stimulated T cell). Regulation of the expression and/or secretion of Designed Cytokines of the disclosure reduced systemic exposure in vivo and provides an advantageous safety profile for the Designed Cytokines.

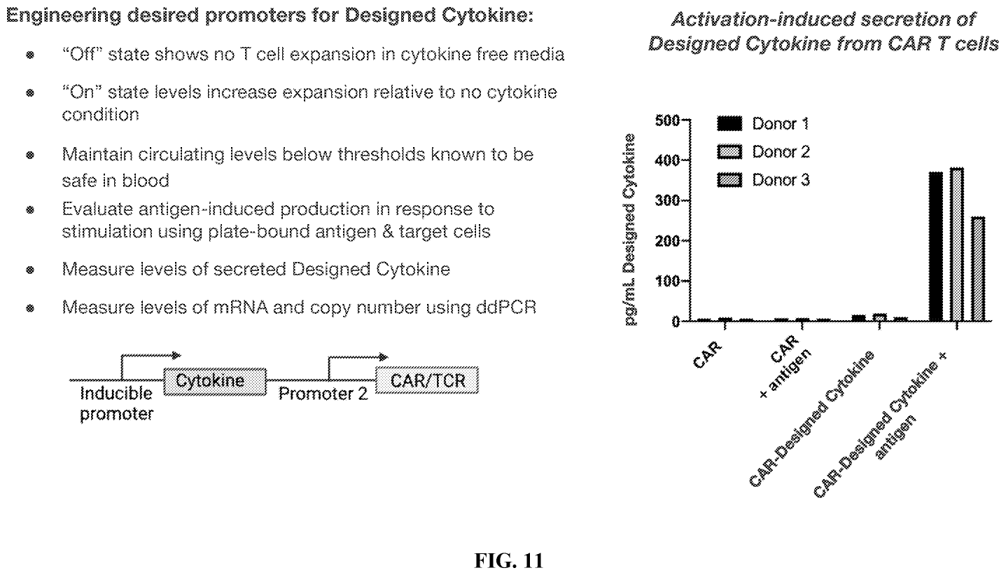

FIG. 12 is a schematic diagram and a graph demonstrating that, when compared to WT IL-2, incorporating short and structured loops within the Designed Cytokines increases the protein stability of the Designed Cytokine. The schematic diagram on the left depicts the structural differences of the loops in WT IL-2 versus the Designed Cytokines of the disclosure. The graph depicts protein unfolding/instability (measured by intrinsic fluorescence) as a function of function of increasing molar concentrations of guanidine hydrochloride ([GdnHCl (M)]), an agent that denatures/unfolds proteins. The greater stability of the Designed Cytokine (circle data points) is illustrated by the lower fluorescence at higher concentrations (right shift) compared to the WT IL-2. As used throughout the disclosure, the label “WT IL-2 internal” is meant to describe a WT IL-2 having an identical sequence to commercially available WT IL-2 proteins, that was synthesized by the applicant.

FIG. 13 is a graph demonstrating that two exemplary Designed Cytokines of the disclosure exhibit IL-15-like effects on NK cells in vitro. In this experiment, the activity of a commercially purchased WT IL-2 and the internally generated WT IL-2 were compared. A WT IL-15 polypeptide was used as a positive control and for a basis of comparison for IL-15-like, as opposed to IL-2-like effects.

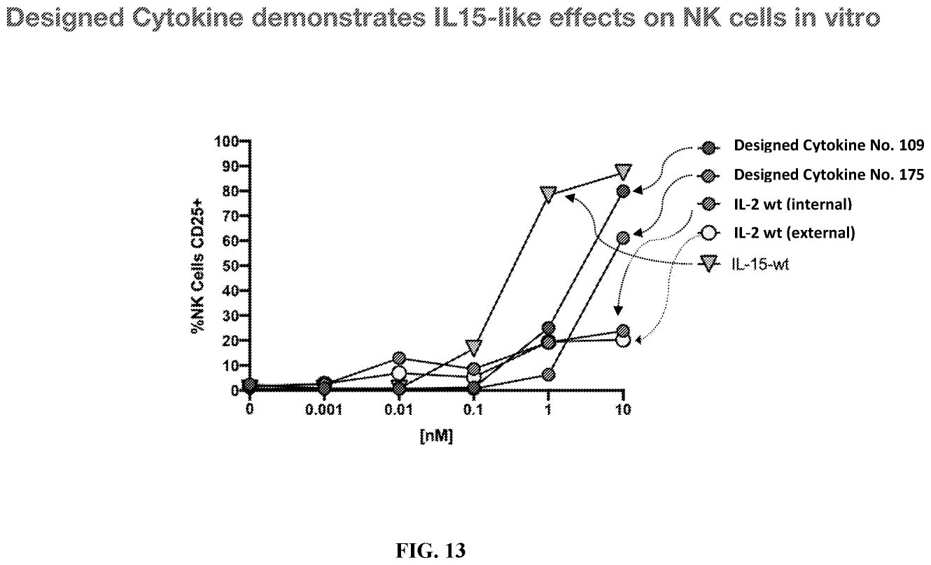

FIG. 14A is a series of schematic diagrams demonstrating that, in some embodiments, Designed Cytokines of the disclosure may be fused to a targeting moiety, or, described from another perspective, a fusion protein may comprise a Designed Cytokine and a targeting moiety. The right-most diagram depicts signaling in trans.

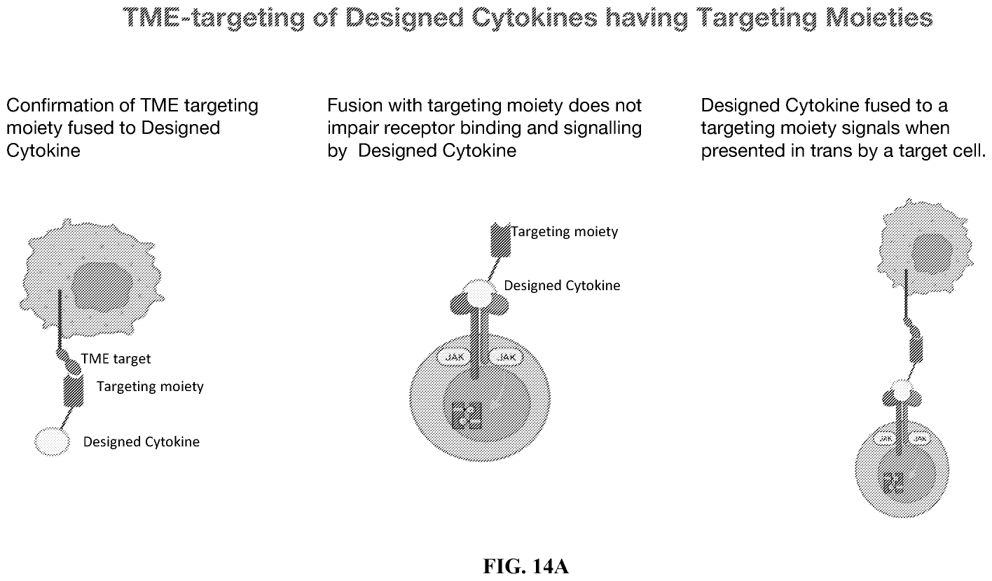

FIG. 14B is a graph demonstrating that the use of the targeting moiety to localize the physical presence and/or signaling activity of the Designed Cytokine to, for example, a TME, does not impair any activity, receptor binding of or signaling from the Designed Cytokine. Within this graph, the percentage of total cells that bind Designed Cytokines are shown as a function of the nanomolar (nM) protein concentration the targeting moiety bound to the Designed Cytokine.

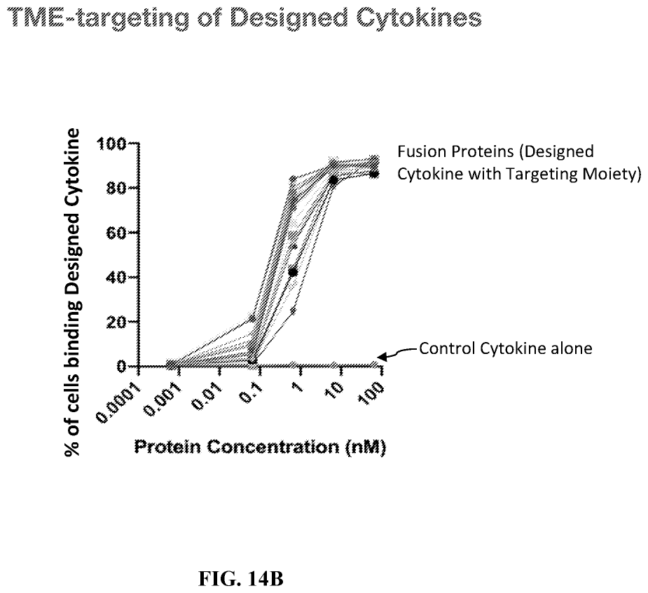

FIG. 14C is a series of graphs demonstrating the use of targeting moieties with four exemplary Designed Cytokines of the disclosure. In each plot, the cell proliferative activity of each Targeted Designed Cytokine is measured as a percentage of phosphorylated PSTAT5+ cells as a function of the nanomolar concentration of the Targeted Designed Cytokine provided. The labels, “VHH-1” as opposed to “VHH-2” are meant to depict two VHH targeting moieties having distinct sequences.

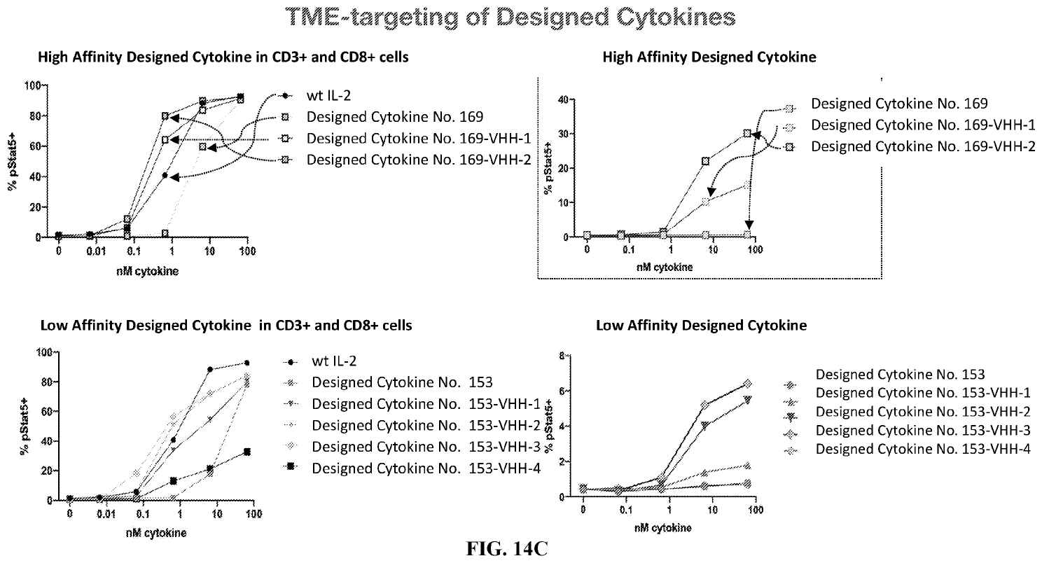

FIG. 15 is a series of schematic diagrams of two exemplary Designed Cytokines of the disclosure, contrasted against WT IL-2, demonstrating the structural differences between IL-2 and each Designed Cytokine. Moreover, the diagrams reflect structural modifications to each Designed Cytokine that result in functional changes to the protein. While each Designed Cytokine contains a helical initiator (e.g., a lysine (L)) at the front to the H4 helix, the “conjugation” Designed Cytokine which more easily conjugates with, for example, a targeting moiety, has an extended H2 helix and a shorter loop between the H4 and H2 helices. Both Designed Cytokines have a substitution at a position three amino acids from the inter-H2 helix loop, albeit, the substitution itself is distinct between these two Designed Cytokines.

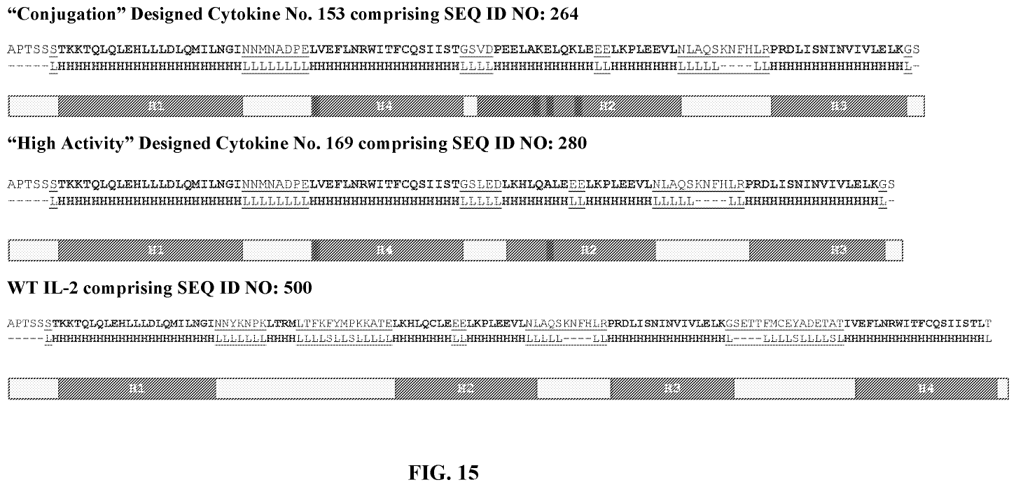

FIG. 16 is a series of graphs demonstrating that Designed Cytokine No. 201 shows no detectable binding to IL2Rα whereas WT IL-2 binds to IL2Rα with nanomolar affinity.

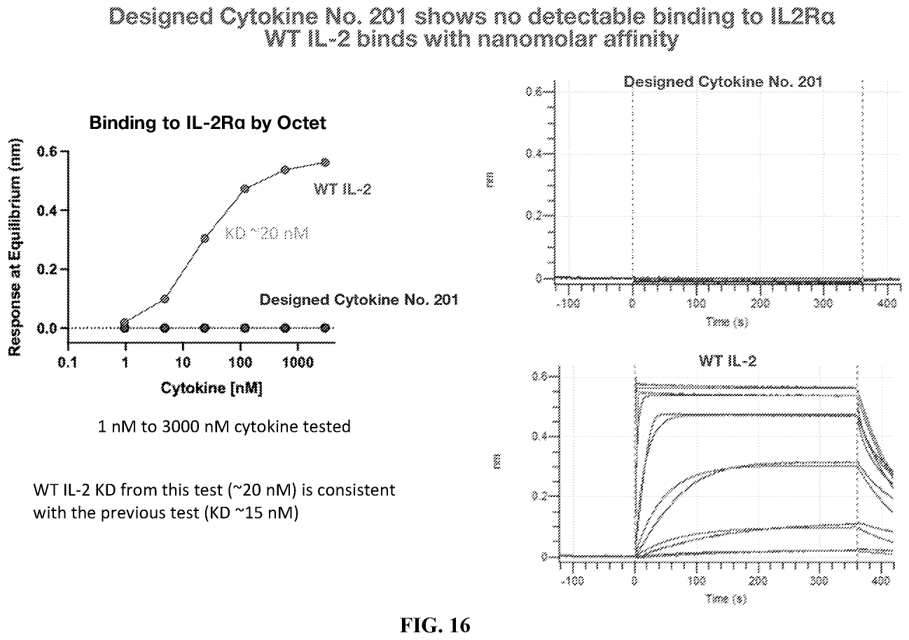

FIG. 17A is a graph depicting two replicate experiments (the previous experiment shown in FIG. 6) side-by-side to demonstrate that activities of exemplary Designed Cytokines may be tuned over a wide range.

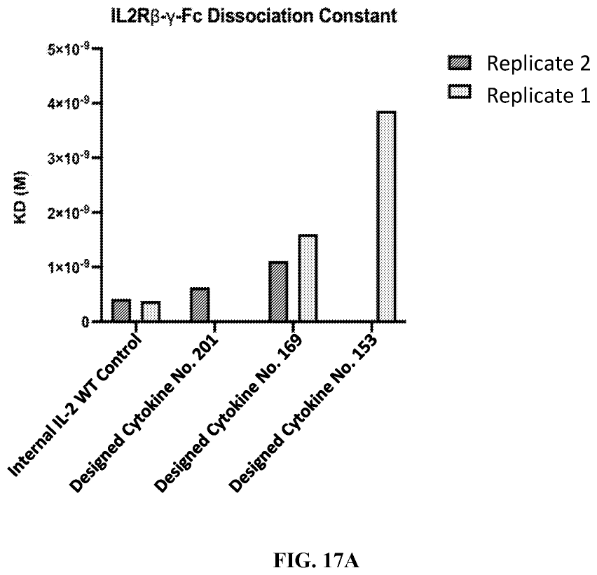

FIG. 17B is a graph depicting the later replicate of the experiments shown in FIG. 6 and FIG. 17A.

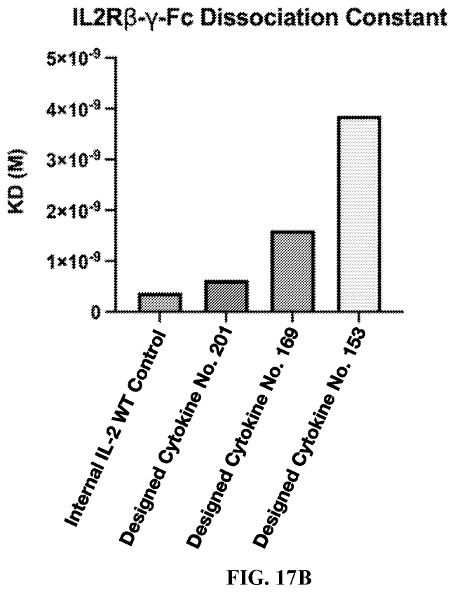

FIG. 18 is a series of graphs demonstrating the binding affinity (by Octet) of either WT IL-2 (left-hand plots) and two exemplary Designed Cytokines (middle and right-hand plots) to IL-2Rβγ.

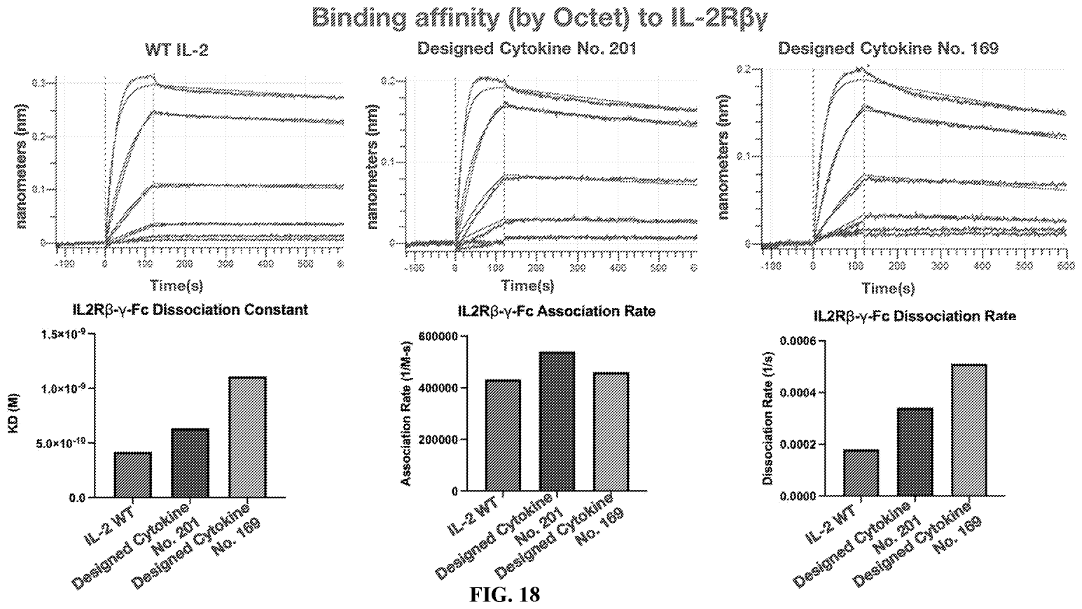

FIG. 19 is a series of graphs demonstrating the cell proliferative capacity of three exemplary Designed Cytokines when compared to WT IL-2 in three cell types (NK cells, Treg cells and CD8+ T cells).

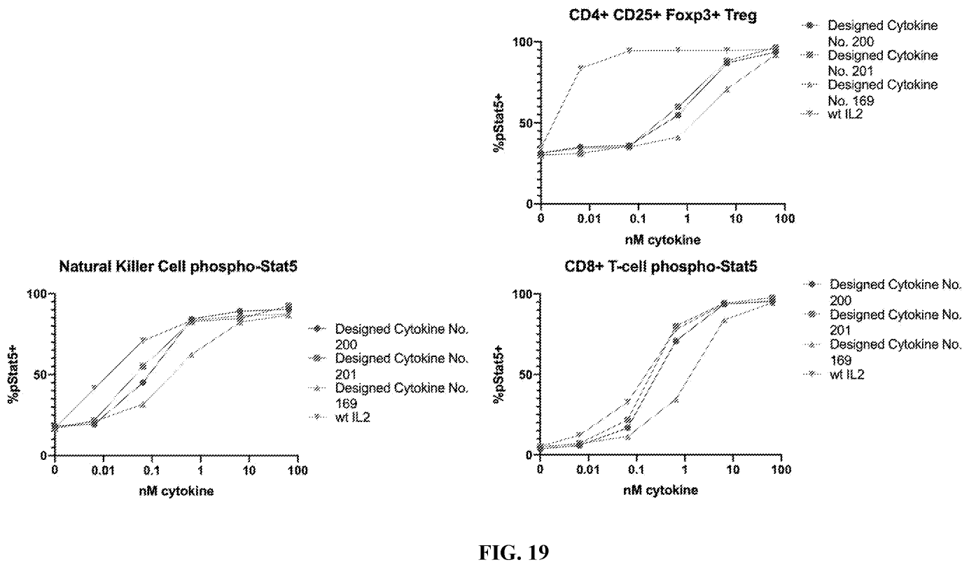

FIG. 20 is a pair of schematic diagrams and coordinating pairs of graphs demonstrating the cis versus trans targeting of Designed Cytokines of the disclosure by varying the binding preferences of the targeting moieties. As shown in the top schematic diagram and top row of graphs, PD-1 and CD8 mediate cis-targeting of activated T cells by Designed Cytokines of the disclosure. As shown in the bottom schematic diagram and bottom row of graphs, PD-L1 mediates cis-targeting on activated cells and trans presentation of Designed Cytokines of the disclosure.

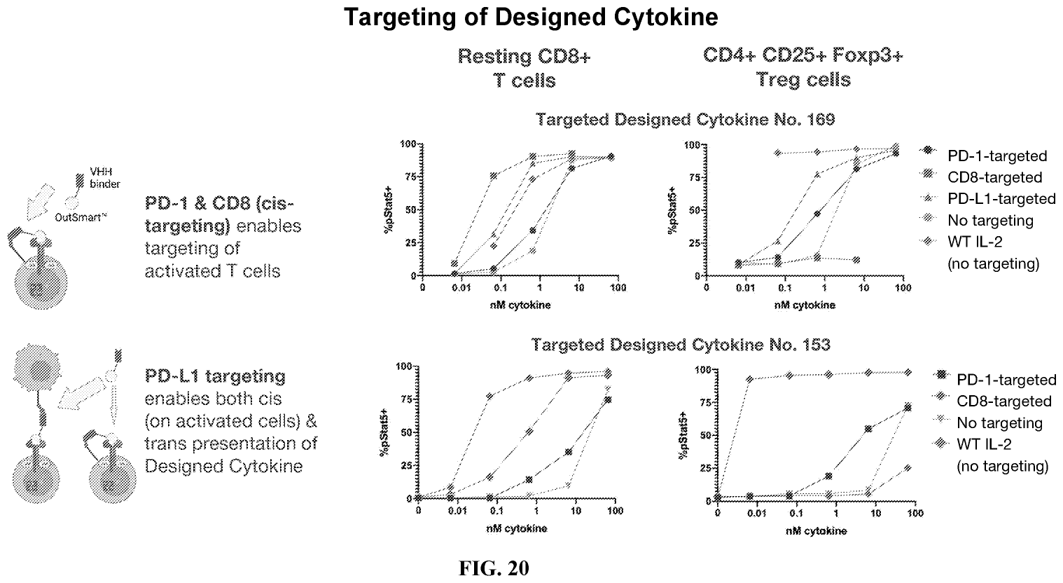

FIG. 21A is a schematic diagram showing the experimental design used to generate data provided in FIGS. 21B and 21C. Briefly, CAR-T cells were sequentially challenged with H1975 tumor cells in the presence of recombinant Designed Cytokine, Designed Cytokine+binding domain to target (added separately at equimolar ratios), or targeted Designed Cytokine: Targeted Designed Cytokine No. 169 is fused to a VHH binding domain that binds either PD-L1, PD-1, or CD8.

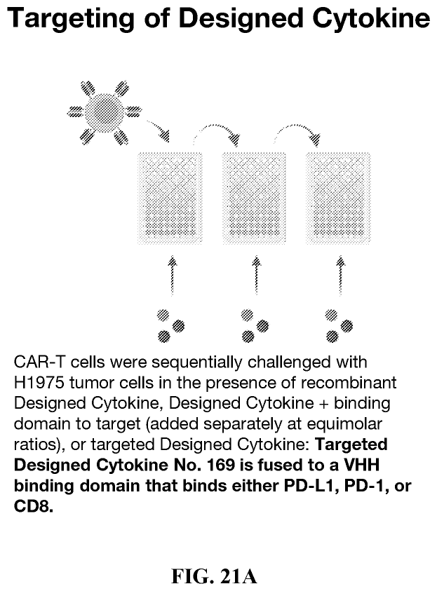

FIG. 21B is a pair of graphs, corresponding to two distinct T-cell donors, demonstrating the killing activity of PDL1-targeted Designed Cytokines of the disclosure.

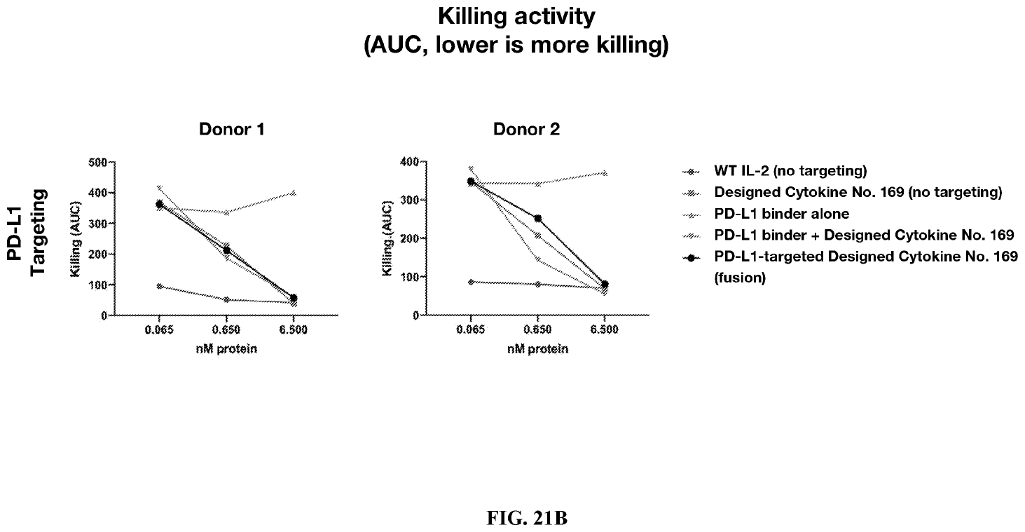

FIG. 21C is a pair of graphs, corresponding to two distinct T-cell donors, demonstrating the killing activity of PDL1-targeted Designed Cytokines of the disclosure.

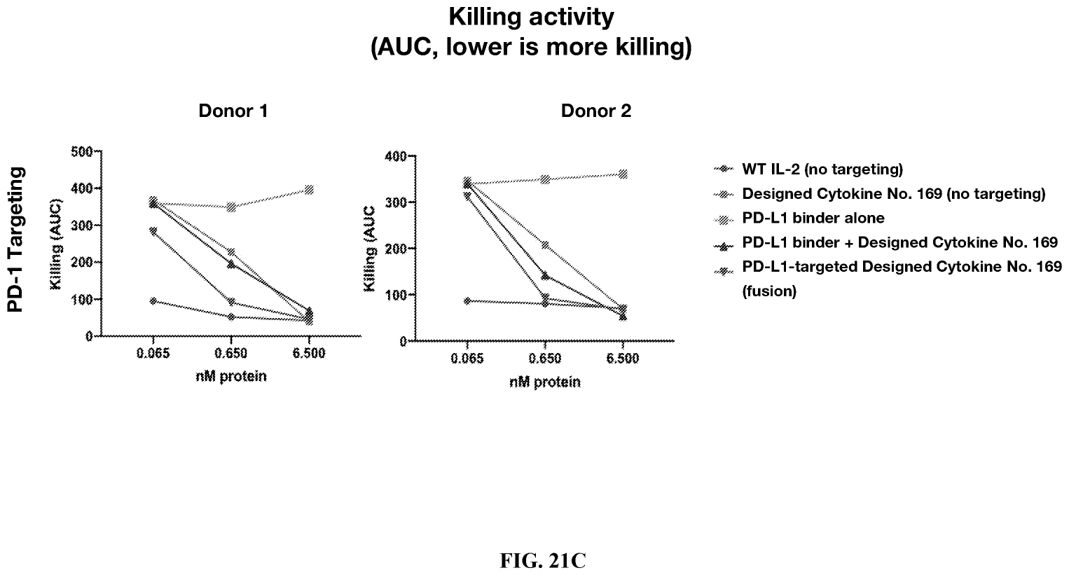

FIG. 21D is a pair of graphs, corresponding to two distinct T-cell donors, demonstrating the killing activity of CD8-targeted Designed Cytokines of the disclosure.

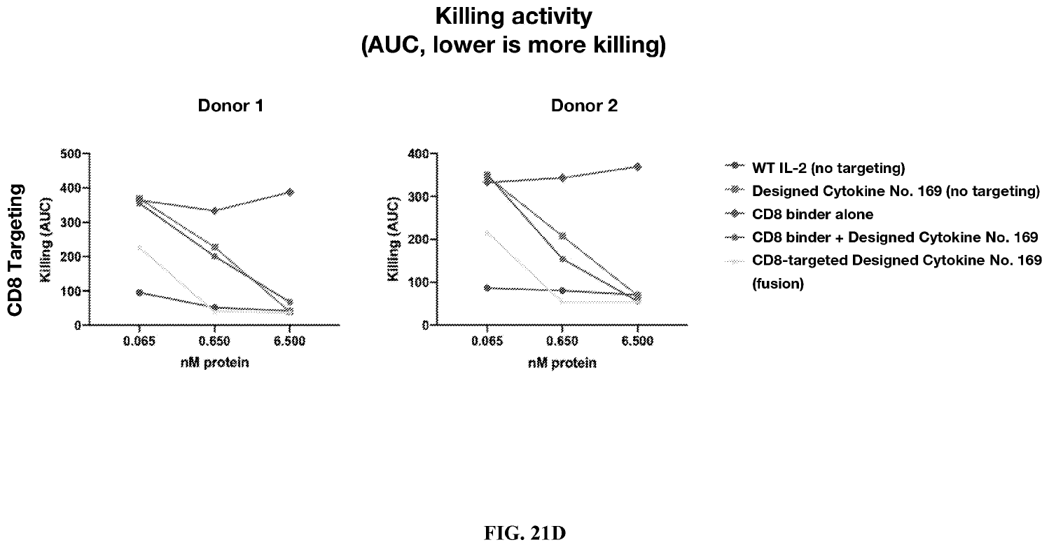

FIG. 22A is a graph demonstrating the cell proliferation activity of Designed Cytokines derived from Designed Cytokine No. 153 in Natural Killer cells.

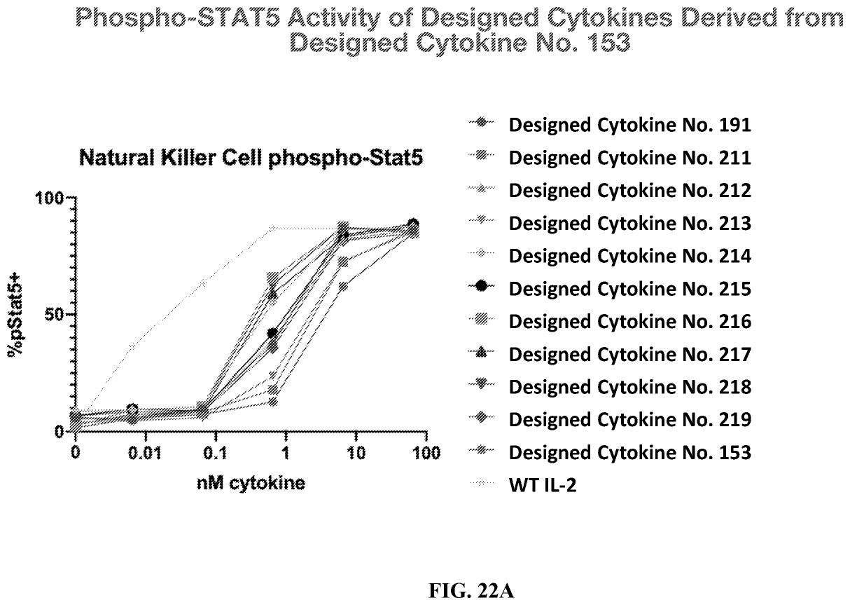

FIG. 22B is a graph demonstrating the cell proliferation activity of Designed Cytokines derived from Designed Cytokine No. 153 in CD8+ T cells.

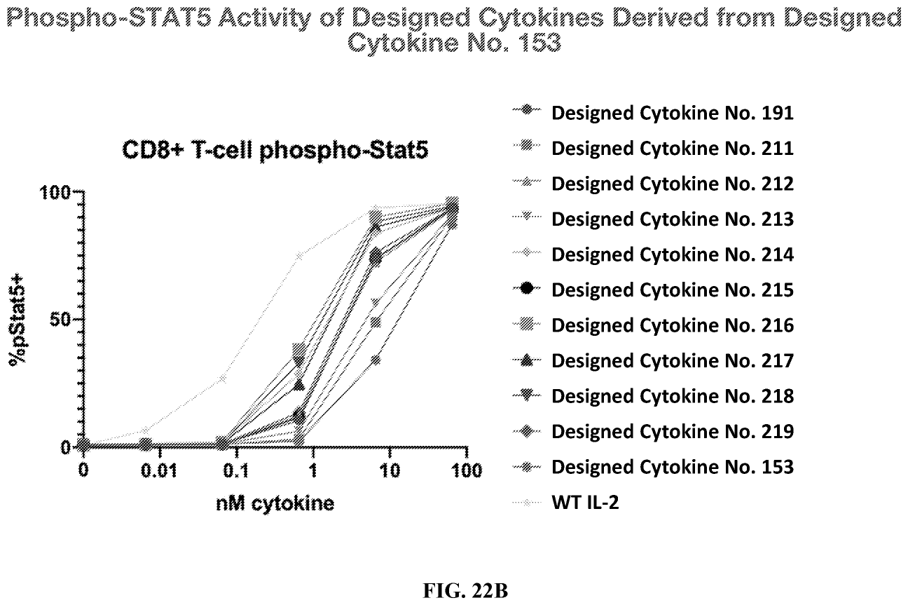

FIG. 23A is a series of schematic diagrams depicting two vector (2V), single vector (1V) and autoregulation circuits incorporating an Inducible Designed Cytokine. In these schematics, the term “DC” is meant to describe a Designed Cytokine. In some embodiments, and in accordance with these diagrams, the expression of an Inducible Designed Cytokine is under the control of one or more response elements (RE Array) and a promoter sequence (which may be a minimal promoter sequence (pMin)). The Marker and the CAR elements in each 2V and 1V diagram are under the control of the MND promoter. In the autoregulation diagram, the expression of the CAR and the Designed Cytokine are under the control of an inducible promoter, which is active during periods of cell stimulation.

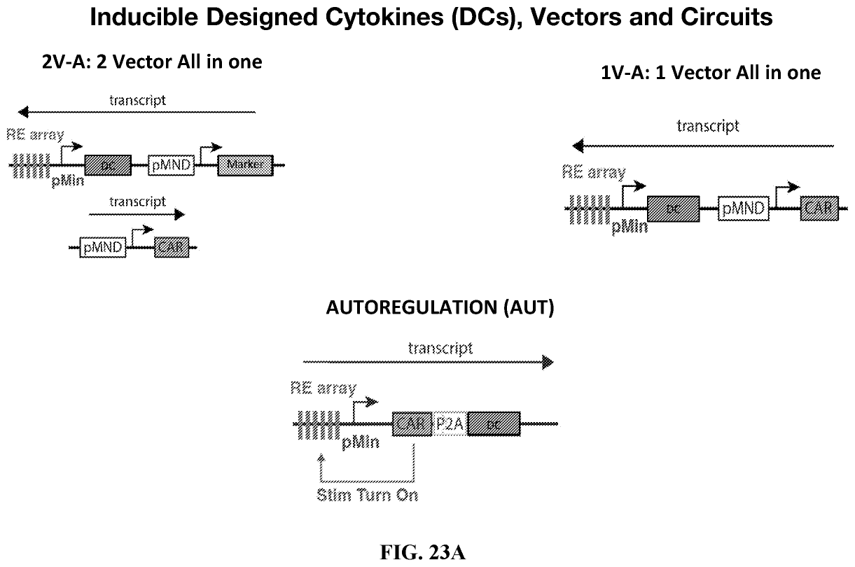

FIG. 23B is a schematic diagram depicting the experimental design that was used to generate the data shown in FIG. 23C. In this experiment, CAR-T cells containing a vector or circuit depicted in 23 A were stimulated by plate-bound antigen. WT IL-2 in the control or the Secreted Inducible Designed Cytokine was measured in the supernatant.

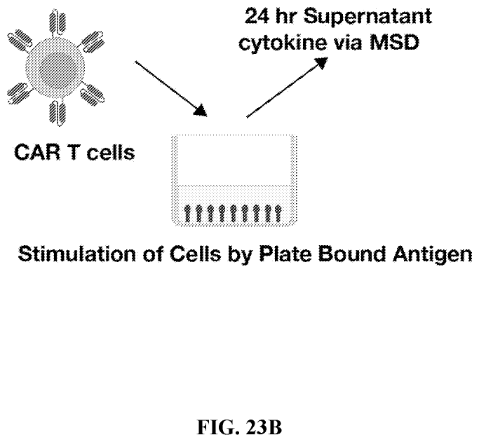

FIG. 23C is a graph demonstrating that Inducible Designed Cytokines (DCs) show increased expression relative to Control, either CAR only (with no Designed Cytokine) or without antigen stimulation.

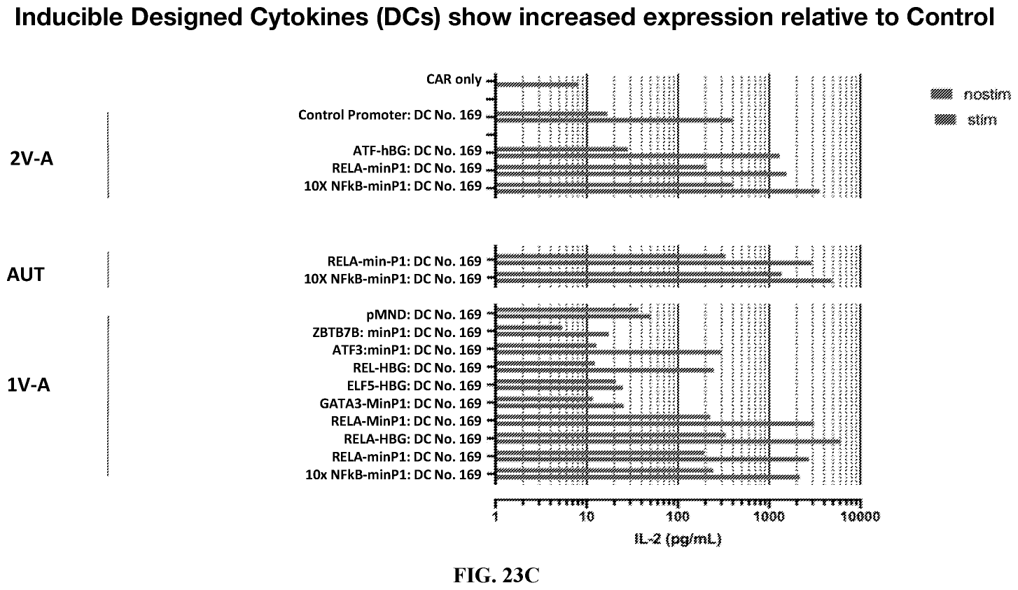

FIG. 24 is a series of graphs demonstrating that exemplary Inducible Designed Cytokines retain target cell killing activity better than a CAR-only control after three sequential rounds of stimulation.

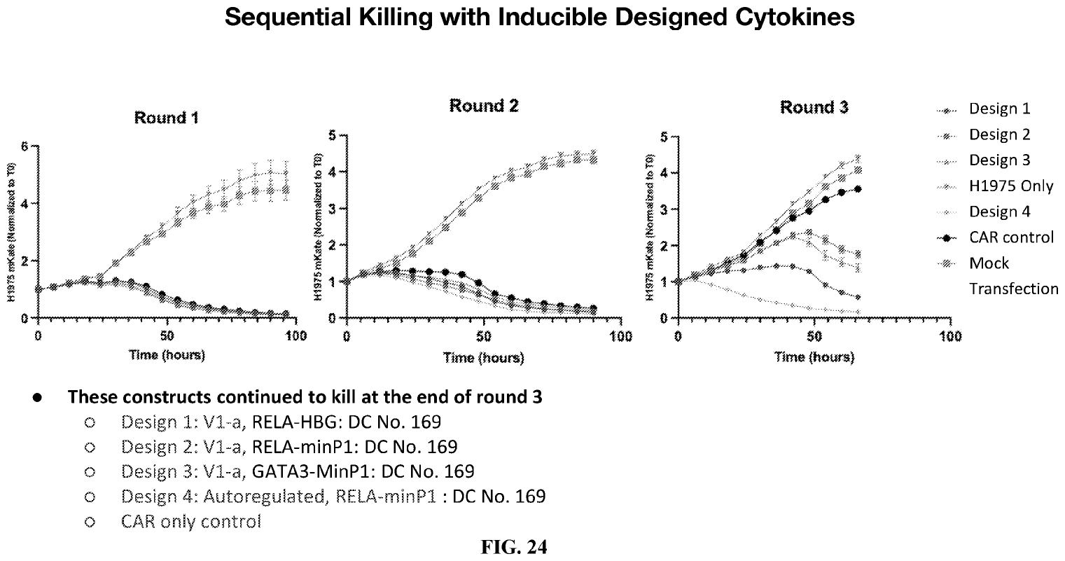

### DETAILED DESCRIPTION

The disclosure provides “designed cytokines” that are non-naturally occurring polypeptides of the disclosure, in accordance with the embodiments illustrated within the drawings and the sequences provided herein, that demonstrate functional benefits of IL-2 and IL-15. Accordingly, the “designed cytokines” may be referred to as “IL-2/15 Polypeptides” or “IL-2/15.” These terms are interchangeable with “designed cytokines.”

The disclosure provides designed cytokines, preferably demonstrating one or more of the following attributes: (1) reduces or ablates binding of the designed cytokine to the alpha subunit of the IL-2 Receptor (IL-2Rα), optionally without post-translational modifications to the designed IL-2 polypeptide, (2) binds or retains binding of the designed IL-2 polypeptide to the beta and/or gamma subunit of the IL-2 Receptor (IL-2Rβ/γ), (3) preferentially stimulates or more potently stimulates T cell(s) expressing CD8 (CD8 T cells) more than stimulating regulatory T cell(s) (Treg), (4) preferentially stimulates or more potently stimulates Natural Killer (NK) cells than CD8 T cells, (5) enhances an activity of T cells expressing or secreting a designed IL-2 polypeptide of the disclosure, (6) demonstrates stable folding of the designed IL-2 polypeptide into a protein in vitro or in vivo, (7) demonstrates reduced, minimal or an undetectable level immunogenicity when administered to a subject (e.g., a mouse, another animal species or a human patient) when compared to the level of immunogenicity demonstrated by a wild type IL-2 protein under the same circumstances. Alternatively, or in addition, designed cytokines of the disclosure may (1) bind to an IL-15 Receptor (IL-15R), a beta subunit, or a gamma subunit thereof and/or (2) compete with an IL-15 cytokine, including WT IL-15, for binding to an IL-15R, a beta subunit, or a gamma subunit thereof.

In some embodiments, designed cytokines of the disclosure (1) localize expression, translation, production and/or secretion of a designed IL-2 polypeptide of the disclosure to a tumor, target cell, and/or tumor microenvironment (TME), (2) target a TME, and/or (3) maintain localization at a TME. In some embodiments of designed cytokines of the disclosure, including those in which the polypeptide targets, localizes and/or maintains localization within a TME, the designed cytokine comprises a targeting moiety. In some embodiments, the targeting moiety comprises a nucleic acid, an amino acid, or a combination thereof which specifically binds to a target on or with the lymph node, the tumor, the tumor microenvironment, the site of malignancy or the site of metastasis, or in each case a cell thereof. In some embodiments, the fusion protein or the targeting moiety comprises an antibody, an antibody mimetic, or a functional fragment thereof. In some embodiments, the targeting moiety comprises an scFv, a VH or a VHH. In some embodiments, the targeting moiety comprises an scFv, a VH or a VHH that specifically or selectively binds to T-cell surface glycoprotein CD8 (also known as cluster of differentiation 8), Programmed cell death protein 1 (PD-1), Programmed death-ligand 1 (PD-L1; also known as cluster of differentiation 274 (CD274) or B7 homolog 1 (B7-H1) polypeptide), T-cell immunoreceptor with Ig and ITIM domains (TIGIT), Cytotoxic T-lymphocyte protein 4 (CTLA4), Lymphocyte activation gene 3 protein (LAG3), T-cell immunoglobulin mucin receptor 3 (TIM3).

In some embodiments of designed cytokines of the disclosure, the designed cytokine comprises a “tether” to operably-link the designed cytokine to (1) a cell expressing the designed cytokine or (2) a cell delivering the designed cytokine to a target cell, an immune synapse and/or a TME. In some embodiments, the tether comprises a DNA, RNA, amino acid or any combination thereof. In some embodiments, the tether comprises a defined secondary structure. In some embodiments, the tether has a rigid structure. In some embodiments, the tether has a flexible conformation. In some embodiments, the tether has an elastic conformation. In some embodiments, the tether comprises a cleavable sequence. In some embodiments, the tether comprises a transmembrane sequence or a membrane anchoring sequence. In some embodiments, the tether is optionally linked to the Designed Cytokine by a linker sequence. In some embodiments, the tether is optionally linked to the Designed Cytokine by a linker sequence comprising a “GS” linker. In some embodiments, the tether comprises a sequence of

“PLFIPVAVMVTAFSGLAFIIWLARRLKKGKK.”

In some embodiments of designed cytokines of the disclosure, a construct comprises an inducible promoter capable of expressing a designed cytokine and a sequence encoding a designed cytokine. In some embodiments, the construct may further comprise a sequence encoding a targeting moiety. Alternatively, or in addition, in some embodiments, the construct may further comprise a sequence encoding a tether. In some embodiments, the construct may further comprise a linker positioned between the sequence encoding the designed cytokine and one or more of a sequence encoding a targeting moiety and a sequence encoding a tether.

In some embodiments of designed cytokines of the disclosure, a fusion protein comprises a designed cytokine and either a targeting moiety or a tether. In some embodiments of the fusion protein, a linker is positioned between the designed cytokine and either the targeting moiety or the tether.

In some embodiments of designed cytokines of the disclosure, an immune cell expresses a designed cytokine of the disclosure. In some embodiments, the immune cell secretes the designed cytokine. In some embodiments, the designed cytokine comprises a tether and contacts to the immune cell. In some embodiments, the tethered designed cytokine contacts a plasma membrane of the immune cell or a component thereof. In some embodiments, the tethered designed cytokine contacts an exterior surface of the plasma membrane of the immune cell.

In some embodiments, designed cytokines of the disclosure are capable of being expressed in a vector alone, or in combination, with one or more of an antigen receptor (e.g., a T cell receptor (TCR), a chimeric antigen receptor (CAR), or any combination thereof). In some embodiments, the vector comprises a viral vector. In some embodiments, the vector comprises a non-viral vector. In some embodiments, the vector consists of a single vector. In some embodiments, a viral vector comprises a sequence comprising a sequence encoding the designed cytokine and a sequence encoding an antigen receptor (e.g., a T cell receptor (TCR), a chimeric antigen receptor (CAR), or any combination thereof). In some embodiments, a single viral vector comprises a sequence comprising a sequence encoding the designed cytokine and a sequence encoding an antigen receptor (e.g., a T cell receptor (TCR), a chimeric antigen receptor (CAR), or any combination thereof). In some embodiments, the viral vector comprises a sequence isolated or derived from a lentiviral vector. In some embodiments, the viral vector comprises a lentiviral vector.

### Function of Designed Cytokines

The disclosure provides designed cytokines demonstrating functional benefits of IL-2 and IL-15. The disclosure provides designed cytokines, preferably demonstrating one or more of the following attributes: (1) reduces or ablates binding of the designed cytokine to the alpha subunit of the IL-2 Receptor (IL-2Rα), optionally without post-translational modifications to the designed IL-2 polypeptide, (2) binds or retains binding of the designed IL-2 polypeptide to the beta and/or gamma subunit of the IL-2 Receptor (IL-2Rβ/γ), (3) preferentially stimulates or more potently stimulates T cell(s) expressing CD8 (CD8 T cells) more than stimulating regulatory T cell(s) (Treg), (4) preferentially stimulates or more potently stimulates Natural Killer (NK) cells than CD8 T cells, (5) enhances an activity of T cells expressing or secreting a designed IL-2 polypeptide of the disclosure, (6) demonstrates stable folding of the designed IL-2 polypeptide into a protein in vitro or in vivo, (7) demonstrates reduced, minimal or an undetectable level immunogenicity when administered to a subject (e.g., a mouse, another animal species or a human patient) when compared to the level of immunogenicity demonstrated by a wild type IL-2 protein under the same circumstances. Alternatively, or in addition, designed cytokines of the disclosure may (1) bind to an IL-15 Receptor (IL-15R), a beta subunit, or a gamma subunit thereof and/or (2) compete with an IL-15 cytokine, including WT IL-15, for binding to an IL-15R, a beta subunit, or a gamma subunit thereof.

Wild type IL-2 and IL-15 both stimulate signaling through IL-2/15Rβ (also referred to IL-2Rβ) and common IL-2/15Rγ chains (also referred to as IL-2Rγ); IL-2 and IL-15 both bind to and activate signaling through the heterodimeric By receptor complex, IL-2/15Rβγ (also referred to as IL-2Rβγ), and their unique biology is principally driven through respective interactions with IL-2Rα (cis presentation) and IL-15Rα (trans presentation). Designed IL-2 polypeptides of the disclosure (also known as IL-2/15 polypeptides of the disclosure) agonize the By receptor pair that is shared by IL-2 and IL-15, while avoiding IL-2Rα and IL-15Ra. Therefore, designed IL-2/15 polypeptides of the disclosure may demonstrate activities of both IL-2 and IL-15 when expressed by different cell types or when contacting different cell types.

IL-2 and IL-15 stimulate diverse types of lymphocytes and natural killer cells. Among the distinct functions between these two cytokines, IL-2 mediates regulatory T cell homeostasis and regulates T helper (TH) differentiation. Moreover, IL-15 mediates expansion of CD8 memory T cells, NK cells, and NK T cells. Designed IL-2/15 polypeptides of the disclosure may demonstrate one or more activities of IL-2 and IL-15 in a particular cell type.

### Tuned or Optimized Designed Cytokines

Designed Cytokines of the disclosure may be “tuned” or “optimized” with respect to an activity of the cytokine. Designed Cytokines of the disclosure may be “tuned” or “optimized” to demonstrate a preferred ratio of an IL-2 activity to an IL-15 activity. In some embodiments, Designed Cytokines may be tuned to achieve particular thresholds of activity of a wild type IL-2 polypeptide and particular thresholds of activity of a wild type IL-15 polypeptide. In some embodiments, tuning of an IL-2/15 polypeptide of the disclosure does not comprise a change to the structure of a Designed Cytokine (e.g., the 1-4-2-3 arrangement of its helices). In some embodiments, tuning of a Designed Cytokine of the disclosure comprises one or more of (1) modifying a sequence of a Designed Cytokine or a portion thereof (e.g., an alpha helix, a loop, or a combination thereof), (2) modifying a physical length of a folded Designed Cytokine or a portion thereof (e.g., an alpha helix, a loop, or a combination thereof), (3) inserting a new sequence into an existing sequence of a Designed Cytokine or a portion thereof (e.g., an alpha helix, a loop, or a combination thereof), and/or (4) removing a sequence of a Designed Cytokine or a portion thereof (e.g., an alpha helix, a loop, or a combination thereof). In some embodiments, a tuned Designed Cytokine of the disclosure comprises a sequence having at least 70%, 75%, 80%, 85%, 90%, 95%, 97%, 99%, or any percentage identity in between when compared to a Designed Cytokine of the disclosure not subjected to the tuning process. In some embodiments, a tuned Designed Cytokine of the disclosure comprises a sequence isolated or derived from an IL-15 sequence.

The disclosure provides Designed Cytokine (formerly known as “IL-2/15 polypeptides”) that may be “tuned” or “optimized” with respect to any activity of the cytokine, to demonstrate a threshold of activity in one or more cell types. In some embodiments, tuning or optimizing a Designed Cytokine of the disclosure for use in a cell type does not comprise a change to the structure of the Designed Cytokine (e.g., the 1-4-2-3 arrangement of its helices). In some embodiments, optimizing a Designed Cytokine of the disclosure for use in a cell type comprises one or more of (1) modifying a sequence of a Designed Cytokine or a portion thereof (e.g., an alpha helix, a loop, or a combination thereof), (2) modifying a physical length of a folded Designed Cytokine or a portion thereof (e.g., an alpha helix, a loop, or a combination thereof), (3) inserting a new sequence into an existing sequence of a Designed Cytokine or a portion thereof (e.g., an alpha helix, a loop, or a combination thereof), and/or (4) removing a sequence of a Designed Cytokine or a portion thereof (e.g., an alpha helix, a loop, or a combination thereof). In some embodiments, an optimized Designed Cytokine of the disclosure comprises a sequence having at least 70%, 75%, 80%, 85%, 90%, 95%, 97%, 99%, or any percentage identity in between when compared to a Designed Cytokine of the disclosure not subjected to the optimizing process. In some embodiments, an optimized Designed Cytokine of the disclosure comprises a sequence isolated or derived from an IL-15 sequence.

### Combinations of Designed Cytokines

In some embodiments of the disclosure, Designed Cytokines may be used in combination with or operably linked to a second cytokine or a second Designed Cytokine. In some embodiments, a Designed Cytokine and a second cytokine or a second Designed Cytokine may be independently modified, regulated and/or targeted. In some embodiments, a Designed Cytokine and a second cytokine or a second Designed Cytokine may be coordinated in one or more of modification, regulation and/or targeting.

In some embodiments, a Designed Cytokine of the disclosure may be used in combination with any cytokine, whether naturally occurring or modified. In some embodiments, a Designed Cytokine of the disclosure may be used in combination with one or more of an IL-2 polypeptide, an IL-12 polypeptide, an IL-15 polypeptide, an IL-18 polypeptide, an IL-21 polypeptide, an IL-23 polypeptide, and an interferon polypeptide (including, but not limited to, an interferon alpha, beta, gamma and/or omega polypeptide). In some embodiments, a Designed Cytokine of the disclosure may be used in combination with an IL-2 polypeptide. In some embodiments, a Designed Cytokine of the disclosure may be used in combination with an IL-12 polypeptide. In some embodiments, a Designed Cytokine of the disclosure may be used in combination with an IL-15 polypeptide. In some embodiments, a Designed Cytokine of the disclosure may be used in combination with an IL-18 polypeptide. In some embodiments, a Designed Cytokine of the disclosure may be used in combination with an IL-21 polypeptide. In some embodiments, a Designed Cytokine of the disclosure may be used in combination with an IL-23 polypeptide. In some embodiments, a Designed Cytokine of the disclosure may be used in combination with an interferon alpha polypeptide. In some embodiments, a Designed Cytokine of the disclosure may be used in combination with an interferon beta polypeptide. In some embodiments, a Designed Cytokine of the disclosure may be used in combination with an interferon gamma polypeptide. In some embodiments, a Designed Cytokine of the disclosure may be used in combination with an interferon omega polypeptide. In some embodiments, the second cytokine comprises a wild type sequence. In some embodiments, the second cytokine does not comprise a wild type sequence. In some embodiments, the second cytokine comprises one or more modifications to alter an activity of the cytokine towards one or more cytokine receptors.

In some embodiments, a Designed Cytokine of the disclosure may be used in combination with a second Designed Cytokine of the disclosure to generate a combination of a first Designed Cytokine and a second Designed Cytokine.

In some embodiments of the disclosure, a Designed Cytokine may be expressed with a second cytokine or a second Designed Cytokine of the disclosure. In some embodiments, the expression may be simultaneous. In some embodiments, the expression may be sequential. In some embodiments, the expression may be regulated or inducible with the use of an inducible promoter of the disclosure.

In some embodiments of the disclosure, a Designed Cytokine may be secreted with a second cytokine or a second Designed Cytokine of the disclosure. In some embodiments, the secretion may be simultaneous. In some embodiments, the secretion may be sequential. In some embodiments, the secretion may be regulated or inducible with the use of an inducible promoter of the disclosure.

In some embodiments of the disclosure, a Designed Cytokine may be operably linked to a second cytokine or a second Designed Cytokine of the disclosure. In some embodiments, a Designed Cytokine and a second cytokine or a second Designed Cytokine may be operably linked via a linker sequence. In some embodiments, the linker sequence comprises a nucleic acid, an amino acid, a small molecule or any combination thereof. In some embodiments, the linker is rigid. In some embodiments, the linker is flexible. For example, a linker sequence may comprise a “GS” sequence of any length.

In some embodiments, a Designed Cytokine and a second cytokine or a second Designed Cytokine may be operably linked via a targeting moiety of the disclosure. In some embodiments, a Designed Cytokine and a second cytokine or a second Designed Cytokine may be operably linked to the same targeting moiety. In some embodiments, a composition comprising a Designed Cytokine and a second cytokine or a second Designed Cytokine may comprise the same targeting moiety. In some embodiments, a Designed Cytokine may comprise a first targeting moiety and a second cytokine or a second Designed Cytokine may comprise a second targeting moiety. In some embodiments, the first targeting moiety and the second targeting moiety bind the same target. In some embodiments, the first targeting moiety and the second targeting moiety do not bind the same target. In some embodiments, the first targeting moiety and the second targeting moiety are identical. In some embodiments, the first targeting moiety and the second targeting moiety not identical.

In some embodiments, a Designed Cytokine and a second cytokine or a second Designed Cytokine may be operably linked via a tether of the disclosure. In some embodiments, a Designed Cytokine and a second cytokine or a second Designed Cytokine may be operably linked to the same tether. In some embodiments, a composition comprising a Designed Cytokine and a second cytokine or a second Designed Cytokine may comprise the same tether. In some embodiments, a Designed Cytokine may comprise a first tether and a second cytokine or a second Designed Cytokine may comprise a second tether. In some embodiments, the first tether and the second tether bind the same target. In some embodiments, the first tether and the second tether do not bind the same target. In some embodiments, the first tether and the second tether are identical. In some embodiments, the first tether and the second tether not identical.

In some embodiments, a Designed Cytokine is operably linked to one or more of an IL-2 polypeptide, an IL-12 polypeptide, an IL-15 polypeptide, an IL-18 polypeptide, an IL-21 polypeptide, an IL-23 polypeptide, and an interferon polypeptide (including, but not limited to, an interferon alpha, beta, gamma and/or omega polypeptide). In some embodiments, a Designed Cytokine is operably linked to an IL-21 polypeptide.

### Targeted Designed Cytokines

In some embodiments of the disclosure, Designed Cytokines target, localize to or remain active at one or more of a target cell, a target cell type, an organ, a lymph node, a tumor (including, but not limited to, liquid tumors, hematological cancers, and solid tumors), a biological microenvironment, a tumor microenvironment (TME), a site of malignancy, a site of metastasis, a site of vascularization of a tumor, and any combination thereof. In some embodiments, a fusion protein comprises a Designed Cytokine that targets, localizes to or remains active at one or more of a target cell, a target cell type, an organ, a lymph node, a tumor (including, but not limited to, liquid tumors, hematological cancers, and solid tumors), a biological microenvironment, a tumor microenvironment (TME), a site of malignancy, a site of metastasis, a site of vascularization of a tumor, and any combination thereof. In some embodiments, a Designed Cytokine comprises a targeting moiety. In some embodiments, a Designed Cytokine is operably linked to a targeting moiety. In some embodiments, a Designed Cytokine is operably linked to a targeting moiety by one or more of a covalent bond, a noncovalent bond, hybridization, dimerization, complex formation, a linker and a tether. In some embodiments, a Designed Cytokine is operably linked to a targeting moiety by a linker comprising one or more of a nucleic acid sequence, an amino acid sequence, a small molecule (organic or inorganic) and any combination thereof. In some embodiments, a Designed Cytokine is operably linked to a targeting moiety by a tether comprising one or more of a nucleic acid sequence, an amino acid sequence, a small molecule (organic or inorganic) and any combination thereof. In some embodiments, a Designed Cytokine is operably linked to a targeting moiety by a tether that is attached to a component of one or more of a target cell, a target cell type, an organ, a lymph node, a tumor (including, but not limited to, liquid tumors, hematological cancers, and solid tumors), a biological microenvironment, a tumor microenvironment (TME), a site of malignancy, a site of metastasis, a site of vascularization of a tumor, and any combination thereof. In some embodiments, a Designed Cytokine is operably linked to a targeting moiety by a tether that is attached to a component of an immune cell expressing or secreting the Designed Cytokine.

In some embodiments of the disclosure, a targeting moiety comprises a nucleic acid, an amino acid, or a combination thereof which specifically binds to a component of one or more of a target cell, a target cell type, an organ, a lymph node, a tumor (including, but not limited to, liquid tumors, hematological cancers, and solid tumors), a biological microenvironment, a tumor microenvironment (TME), a site of malignancy, a site of metastasis, a site of vascularization of a tumor, and any combination thereof. In some embodiments, a targeting moiety comprises a binding domain, a protein scaffold, an antibody, an antibody mimetic, and/or a functional fragment thereof. In some embodiments, a targeting moiety comprises a sequence, which may be isolated or derived from any species, including but not limited to, human, non-human primate, rodent (including, but not limited to, mouse), and camelid species. In some embodiments, a targeting moiety comprises a sequence, which may be humanized, chimeric, recombinant, non-naturally occurring, modified (for example, to include synthetic nucleic acids or synthetic amino acids), optimized (for example, to reduce immunogenicity and/or aggregation during manufacturing).

In some embodiments of the disclosure, a targeting moiety comprises an antibody, including, but not limited to, a monoclonal antibody, an antigen-binding fraction (Fab), a single-chain variable fraction (scFv), a domain antibody, one or more of a heavy chain (VH) and a light chain (VL) domain of an immunoglobulin (Ig) polypeptide or gene encoding the same, a heavy-chain antibody (a VH or a VHH), a camelid or camelid-like structured antibody, and a nanobody. In some embodiments, a targeting moiety comprises an scFv, a VH or a VHH. In some embodiments, a targeting moiety comprises an scFv, a VH or a VHH that specifically or selectively binds to a target including, but not limited to, T-cell surface glycoprotein CD8 (also known as cluster of differentiation 8), Programmed cell death protein 1 (PD-1), Programmed death-ligand 1 (PD-L1; also known as cluster of differentiation 274 (CD274) or B7 homolog 1 (B7-H1) polypeptide), T-cell immunoreceptor with Ig and ITIM domains (TIGIT), Cytotoxic T-lymphocyte protein 4 (CTLA4), Lymphocyte activation gene 3 protein (LAG3), and T-cell immunoglobulin mucin receptor 3 (TIM3).

In some embodiments of the disclosure, a targeting moiety comprises an antibody mimetic, including, but not limited to, one or more of an engineered protein scaffold, a monobody, an affibody molecule, an adnectin molecule, an affimer molecule, an affitin molecule, an affilin molecule, an alphabody molecule, an anticalin molecule, an aptamer molecule, an atrimer molecule, an avimer molecule, a DARPin molecule, a fynomer, an armadillo repeat protein molecule, a Kunitz domain inhibitor molecule, a knottin molecule, a designed ankyrin repeat protein molecule, a nanofittin molecule and a centyrin molecule. In some embodiments, a targeting moiety comprises an antibody mimetic that specifically or selectively binds to a target including, but not limited to, T-cell surface glycoprotein CD8 (also known as cluster of differentiation 8), Programmed cell death protein 1 (PD-1), Programmed death-ligand 1 (PD-L1; also known as cluster of differentiation 274 (CD274) or B7 homolog 1 (B7-H1) polypeptide), T-cell immunoreceptor with Ig and ITIM domains (TIGIT), Cytotoxic T-lymphocyte protein 4 (CTLA4), Lymphocyte activation gene 3 protein (LAG3), and T-cell immunoglobulin mucin receptor 3 (TIM3).

### Inducible Designed Cytokines

The disclosure provides a nucleic acid encoding a non-naturally occurring polypeptide of the disclosure. In some embodiments, a promoter or an inducible promoter capable of driving expression in a mammalian cell controls expression of the nucleic acid encoding a Designed Cytokine of the disclosure.

In some embodiments, an inducible promoter of the disclosure comprises a minimal promoter. In some embodiments, a minimal promoter of the disclosure comprises a sequence isolated or derived from one or more of minimal promoter-1 (“minP1”), YB-TATA and human beta globin. In some embodiments, the minimal promoter comprises one or more of “minP1” having a sequence of AGAGGGTATATAAAAGCTCGACTTCCAG, “minP2” having a sequence of TAGAGGGTATATAATGGGGGCCACTAGTCTACTACCAGAAAGCTTGGTACCGAGCT CGGATCCAGCCACC and “minP3” having a sequence of CTAGAGGGTATATAATGGGGGCCACTAGTCTACTACCAGAAAGCTTGGTACCGAGC TCGGATCCAGCCACC.

In some embodiments, an inducible promoter of the disclosure comprises a sequence isolated or derived from a coding or noncoding sequence of a gene related to one or more of BACH2, BARX1, BATF, ELF1, ELF2, Elf4, Elk1, ERF, ETV1, Fli1, FOXP1, GABPA, GATA3, IRF1, IRF2, IRF5, IRF7, IRF9, MAF, MAFF, Maz, Mef2d, MLX, MYB, NFAT, NFATC3, NFkB, NR4A1, Nur77, PATZ1, REL, RELA, RORa, RORg, RORgt, STAT2, Tbox, TFEB, TOX, USF1, ZBTB2, ZKSCAN3, ZNF12, ZNF140, ZNF263, ZNF282, ZNF304, ZNF398, ZNF708, and ZNF75D. In some embodiments, an inducible promoter of the disclosure comprises a sequence isolated or derived from a coding or noncoding sequence of a gene related to NFAT, NFkB, REL, RELA, IRF2, GATA3 and ATF3. In some embodiments, an inducible promoter of the disclosure comprises a sequence isolated or derived from a coding or noncoding sequence of a gene related to NFAT. In some embodiments, an inducible promoter of the disclosure comprises a sequence isolated or derived from a coding or noncoding sequence of a gene related to NFkB. In some embodiments, an inducible promoter of the disclosure comprises a sequence isolated or derived from a coding or noncoding sequence of a gene related to REL. In some embodiments, an inducible promoter of the disclosure comprises a sequence isolated or derived from a coding or noncoding sequence of a gene related to RELA. In some embodiments, an inducible promoter of the disclosure comprises a sequence isolated or derived from a coding or noncoding sequence of a gene related to IRF2. In some embodiments, an inducible promoter of the disclosure comprises a sequence isolated or derived from a coding or noncoding sequence of a gene related to GATA3. In some embodiments, an inducible promoter of the disclosure comprises a sequence isolated or derived from a coding or noncoding sequence of a gene related to ATF3.

In some embodiments, an inducible promoter of the disclosure comprises a response element and/or an enhancer sequence. In some embodiments, the response element and/or an enhancer sequence comprises a sequence isolated or derived from a coding or noncoding sequence of a gene related to one or more of BACH2, BARX1, BATF, ELF1, ELF2, Elf4, Elk1, ERF, ETV1, Fli1, FOXP1, GABPA, GATA3, IRF1, IRF2, IRF5, IRF7, IRF9, MAF, MAFF, Maz, Mef2d, MLX, MYB, NFAT, NFATC3, NFkB, NR4A1, Nur77, PATZ1, REL, RELA, RORa, RORg, RORgt, STAT2, Tbox, TFEB, TOX, USF1, ZBTB2, ZKSCAN3, ZNF12, ZNF140, ZNF263, ZNF282, ZNF304, ZNF398, ZNF708, and ZNF75D. In some embodiments, the response element and/or an enhancer sequence comprises a sequence isolated or derived from a coding or noncoding sequence of a gene related to one or more of NFAT, NFkB, REL, RELA, IRF2, GATA3 and ATF3. In some embodiments, the response element and/or an enhancer sequence comprises a sequence isolated or derived from NFAT. In some embodiments, the response element and/or an enhancer sequence comprises a sequence isolated or derived from NFkB. In some embodiments, the response element and/or an enhancer sequence comprises a sequence isolated or derived from REL. In some embodiments, the response element and/or an enhancer sequence comprises a sequence isolated or derived from RELA. In some embodiments, the response element and/or an enhancer sequence comprises a sequence isolated or derived from IRF2. In some embodiments, the response element and/or an enhancer sequence comprises a sequence isolated or derived from GATA3. In some embodiments, the response element and/or an enhancer sequence comprises a sequence isolated or derived from ATF3.

In some embodiments, an inducible promoter of the disclosure comprises two or more response elements and/or an enhancer sequences. In some embodiments, an inducible promoter of the disclosure comprises at least 2, 3, 4, 5, 6, 7, 8, 9, or 10 response elements and/or an enhancer sequences. In some embodiments, the repeated response elements and/or enhancer sequences are identical. In some embodiments, the repeated response elements and/or enhancer sequences are not identical.

In some embodiments, an inducible promoter of the disclosure does not comprise the combination of: (1) a Nuclear factor of activated T-cells (NFAT) sequence, a Interferon Regulatory Factor 4 (IRF4) sequence, a activating protein 1 (AP-1)-IRF composite elements (AICE) sequence, or a Interferon Stimulation Response Element (ISRE) sequence; and (2) a human beta globin sequence. In some embodiments, an inducible promoter of the disclosure does not comprise the sequence of GGAGGAAAAACTGTTTCATACAGAAGGCGTGGAGGAAAAACTGTTTCATACAGAA GGCGTGGAGGAAAAACTGTTTCATACAGAAGGCGTGGAGGAAAAACTGTTTCATAC AGAAGGCGTGAATTCAGGGCTGGGCATAAAAGTCAGGGCAGAGCCATCTATTGCTT ACATTTGCTTCTGACACAACTGTGTTCACTAGCAACCTCAAACAGACACC. As used throughout the disclosure, an AICE sequence comprises or consists of sequences isolated or derived from IRF4 or IRF8, each with BATF. In some embodiments, the AICE sequences may be derived from the untranslated region of IRF4, IRF8, and/or BATF. As used throughout the disclosure, an ISRE sequence comprises a consensus sequence of “YAGTTTC(A/T)YTTTYCC” in which “Y” is either C or T.

In some embodiments, an inducible promoter of the disclosure does not comprise the combination of: (1) an NFAT sequence and (2) a YB-TATA sequence. In some embodiments, an inducible promoter of the disclosure does not comprise the sequence of

GGAGGAAAAACTGTTTCATACAGAAGGCGTGGAGGAAAAACTGTTTCAT
ACAGAAGGCGTGGAGGAAAAACTGTTTCATACAGAAGGCGTGGAGGAAA
AACTGTTTCATACAGAAGGCGTGAATTCCTAGAGGGTATATAATGGGGG
CCACTAGTCTACTACCAGAAAGCTTGGTACCGAGCTCGGATCCAGCCAC
C.

In some embodiments, an inducible promoter of the disclosure comprises one or more transcription factor binding motif(s). In some embodiments, an inducible promoter of the disclosure comprises a concatemer of two or more repeated sequences, wherein the repeated sequences may comprise the one or more transcription factor binding motif(s). In some embodiments, the two or more repeated sequences are identical. In some embodiments, the two or more repeated sequences are not identical. In some embodiments, a concatemer comprises a linking sequence positioned between the repeated sequences. In some embodiments, the linking sequence may comprise one or more of: TACGCT, TGATCT, TGCTTT, and TGCCCGT.

In some embodiments, an inducible promoter of the disclosure comprising a concatemer binds more than one unique transcription factor. Alternatively, or in addition, in some embodiments, an inducible promoter of the disclosure comprising a concatemer binds the same transcription factor at more than one site.

In some embodiments, an inducible promoter of the disclosure comprising one or more transcription factor binding motifs, optionally, organized as a concatemer, comprises a sequence according to the consensus sequences provided in Table 16.

In some embodiments, an inducible promoter of the disclosure comprising one or more transcription factor binding motifs, optionally, organized as a concatemer, comprises a sequence selected from any one or more of the sequences in Table 18.

In some embodiments, an inducible promoter of the disclosure comprising a concatemer comprises a sequence according to any one or more of the sequences of Table 19.

In some embodiments, an inducible promoter of the disclosure comprises a pairing of an enhancer sequence and a promoter sequence (an E-P Pairing). In some embodiments, an inducible promoter of the disclosure comprising one or more E-P Pairings comprises a sequence of Table 17.

In some embodiments, designed cytokines of the disclosure are operably coupled to any of the nucleic acid constructs disclosed in U.S. Provisional Application Nos. 63/479,176, 63/479,177, and 63/479,178, which are hereby incorporated by reference in their entireties.

### Structure of Designed Cytokines

The disclosure provides a non-naturally occurring Designed Cytokine (formerly referred to as aninterleukin-2/15 (IL-2/15) polypeptide) comprising alpha helices H1, H2, H3, and H4, wherein, from an amino terminus to a carboxy terminus, a first loop (L1) connects H1 and H4; a second loop (L2) connects H4 and H2; a third loop (L3) connects H2 and H3. In some embodiments, the Designed Cytokine binds to the beta (β) and/or gamma (γ) subunits of the IL-2 receptor. In some embodiments, the Designed Cytokine binds to IL-2 receptor β/γ heterodimer (IL-2Rβ/γ) as a heterodimeric receptor.

In some embodiments of the Designed Cytokines of the disclosure, the Designed Cytokine comprises one or more of a sequence of SEQ ID NO: 1-350.

In some embodiments of the Designed Cytokines of the disclosure, the Designed Cytokine comprises one or more of a sequence of SEQ ID NO: 1-38 or 150-350.

In some embodiments of the Designed Cytokines of the disclosure, the Designed Cytokine comprises a sequence isolated or derived from an IL-2 polypeptide. In some embodiments, the IL-2 polypeptide is a wild type polypeptide. In some embodiments, the IL-2 polypeptide comprises the sequence of SEQ ID NO: 500 below. In some embodiments, a Designed Cytokine of the disclosure may be used combination with an IL-2 polypeptide, including an IL-2 polypeptide having the sequence of SEQ ID NO: 500 below.

Native human (hIL-2) comprises the sequence of (SEQ ID NO: 500):

| | |
|---|---|
| 1 | APTSSSTKKT QLQLEHLLLD LQMILNGINN YKNPKLTRML TFKFYMPKKA TELKHLQCLE |
| 61 | EELKPLEEVL NLAQSKNFHL RPRDLISNIN VIVLELKGSE TTFMCEYADE TATIVEFLNR |
| 121 | WITFCQSIIS TLT. |

hIL-2 has four helices connected by long irregular loops. The N-terminal helix (H1) interacts with both the beta and gamma subunits, the third helix (H3) interacts with the beta subunit, and the C-terminal helix (H4) with the gamma subunit; the alpha subunit interacting surface is formed by the irregular second helix (H2) and two long loops, one connecting H1 to H2 and the other connecting H3 and H4.

In some embodiments of the Designed Cytokines of the disclosure, the Designed Cytokine comprises a sequence isolated or derived from an IL-15 polypeptide. In some embodiments, the IL-15 polypeptide is a wild type polypeptide. In some embodiments, the IL-15 polypeptide comprises the sequence of SEQ ID NO: 501 below. In some embodiments, a Designed Cytokine of the disclosure may be used combination with an IL-15 polypeptide, including an IL-15 polypeptide having the sequence of SEQ ID NO: 501 below.

Native human IL-15 (hIL-15) comprises the sequence of (UniProtKB Accession No. P40933 and SEQ ID NO: 501; signal sequence; IL-15 chain; glycosylation site; disulfide bond):

| | |
|---|---|
| 1 | MRISKPHLRS ISIQCYLCLL LNSHFLTEAG IHVFILGCFS AGLPKTEANW VNVISDLKKI |
| 61 | EDLIQSMHID ATLYTESDVH PSCKVTAMKC FLLELQVISL ESGDASIHDT VENLIILANN |
| 121 | SLSSNGNVTE SGCKECEELE EKNIKEFLQS FVHIVQMFIN TS. |

hIL-15 has a similar structure to hIL-2, however hIL-15 has less than 20% sequence identity with hIL-2.

In some embodiments, a Designed Cytokine of the disclosure may be used combination with an IL-12 polypeptide, including an IL-12 polypeptide, subunit A, having the sequence of MCPARSLLLVATLVLLDHLSLARNLPVATPDPGMFPCLHHSQNLLRAVSNMLQKARQT LEFYPCTSEEIDHEDITKDKTSTVEACLPLELTKNESCLNSRETSFITNGSCLASRKTSFM MALCLSSIYEDLKMYQVEFKTMNAKLLMDPKRQIFLDQNMLA VIDELMQALNFNSETV PQKSSLEEPDFYKTKIKLCILLHAFRIRAVTIDRVMSYLNAS. In some embodiments, a Designed Cytokine of the disclosure may be used combination with an IL-12 polypeptide, including an IL-12 polypeptide, subunit B, having the sequence of

MCHQQLVISWFSLVFLASPLVAIWELKKDVYVVELDWYPDAPGEMVVLT
CDTPEEDGITWTLDQSSEVLGSGKTLTIQVKEFGDAGQYTCHKGGEVLS
HSLLLLHKKEDGIWSTDILKDQKEPKNKTFLRCEAKNYSGRFTCWWLTT
ISTDLTFSVKSSRGSSDPQGVTCGAATLSAERVRGDNKEYEYSVECQED
SACPAAEESLPIEVMVDAVHKLKYENYTSSFFIRDIIKPDPPKNLQLKP
LKNSRQVEVSWEYPDTWSTPHSYFSLTFCVQVQGKSKREKKDRVFTDKT
SATVICRKNASISVRAQDRYYSSSWSEWASVPCS.

In some embodiments, a Designed Cytokine of the disclosure may be used combination with an IL-18 polypeptide, including an IL-18 polypeptide, having the sequence of

MAAEPVEDNCINFVAMKFIDNTLYFIAEDDENLESDYFGKLESKLSVIR
NLNDQVLFIDQGNRPLFEDMTDSDCRDNAPRTIFIISMYKDSQPRGMAV
TISVKCEKISTLSCENKIISFKEMNPPDNIKDTKSDIIFFQRSVPGHDN
KMQFESSSYEGYFLACEKERDLFKLILKKEDELGDRSIMFTVQNED.

In some embodiments, a Designed Cytokine of the disclosure may be used combination with an IL-21 polypeptide, including an IL-21 polypeptide, having the sequence of

MRSSPGNMERIVICLMVIFLGTLVHKSSSQGQDRHMIRMRQLIDIVDQL
KNYVNDLVPEFLPAPEDVETNCEWSAFSCFQKAQLKSANTGNNERIINV
SIKKLKRKPPSTNAGRRQKHRLTCPSCDSYEKKPPKEFLERFKSLLQKM
IHQHLSSRTHGSEDS.

In some embodiments, a Designed Cytokine of the disclosure may be used combination with an IL-23 polypeptide, including an IL-23 polypeptide, subunit A, having the sequence of MLGSRAVMLLLLLPWTAQGRAVPGGSSPAWTQCQQLSQKLCTLAWSAHPLVGHMDL REEGDEETTNDVPHIQCGDGCDPQGLRDNSQFCLQRIHQGLIFYEKLLGSDIFTGEPSLLP DSPVGQLHASLLGLSQLLQPEGHHWETQQIPSLSPSQPWQRLLLRFKILRSLQAFVAVA ARVFAHGAATLSP. In some embodiments, a Designed Cytokine of the disclosure may be used combination with an IL-23 polypeptide, including an IL-23 polypeptide, subunit B, having the sequence of

MCHQQLVISWFSLVFLASPLVAIWELKKDVYVVELDWYPDAPGEMVVLT
CDTPEEDGITWTLDQSSEVLGSGKTLTIQVKEFGDAGQYTCHKGGEVLS
HSLLLLHKKEDGIWSTDILKDQKEPKNKTFLRCEAKNYSGRFTCWWLTT
ISTDLTFSVKSSRGSSDPQGVTCGAATLSAERVRGDNKEYEYSVECQED
SACPAAEESLPIEVMVDAVHKLKYENYTSSFFIRDIIKPDPPKNLQLKP
LKNSRQVEVSWEYPDTWSTPHSYFSLTFCVQVQGKSKREKKDRVFTDKT
SATVICRKNASISVRAQDRYYSSSWSEWASVPCS.

In some embodiments, a Designed Cytokine of the disclosure may be used combination with an interferon alpha polypeptide, including an interferon alpha 1/13, polypeptide having the sequence of MASPFALLMVLVVLSCKSSCSLGCDLPETHSLDNRRTLMLLAQMSRISPSSCLMDRHDF GFPQEEFDGNQFQKAPAISVLHELIQQIFNLFTTKDSSAAWDEDLLDKFCTELYQQLNDL EACVMQEER VGETPLMNADSILAVKKYFRRITLYLTEKKYSPCA WEVVRAEIMRSLSLS TNLQERLRRKE. In some embodiments, a Designed Cytokine of the disclosure may be used combination with an interferon alpha polypeptide, including an interferon alpha 2, polypeptide having the sequence of MALTFALLVALLVLSCKSSCSVGCDLPQTHSLGSRRTLMLLAQMRRISLFSCLKDRHDF GFPQEEFGNQFQKAETIPVLHEMIQQIFNLFSTKDSSAAWDETLLDKFYTELYQQLNDLE ACVIQGVGVTETPLMKEDSILA VRKYFQRITLYLKEKKYSPCAWEVVRAEIMRSFSLST NLQESLRSKE. In some embodiments, a Designed Cytokine of the disclosure may beused combination with an interferon alpha polypeptide, including an interferon alpha 4, polypeptide having the sequence of MALSFSLLMAVLVLSYKSICSLGCDLPQTHSLGNRRALILLAQMGRISHFSCLKDRHDF GFPEEEFDGHQFQKAQAISVLHEMIQQTFNLFSTEDSSAAWEQSLLEKFSTELYQQLNDL EACVIQEVGVEETPLMNEDSILA VRKYFQRITLYLTEKKY SPCA WEVVRAEIMRSLSFST NLQKRLRRKD. In some embodiments, a Designed Cytokine of the disclosure may be used combination with an interferon alpha polypeptide, including an interferon alpha 5, polypeptide having the sequence of MALPFVLLMALVVLNCKSICSLGCDLPQTHSLSNRRTLMIMAQMGRISPFSCLKDRHDF GFPQEEFDGNQFQKAQAISVLHEMIQQTFNLFSTKDSSATWDETLLDKFYTELYQQLND LEACMMQEVGVEDTPLMNVDSILTVRKYFQRITLYLTEKKYSPCAWEVVRAEIMRSFS LSANLQERLRRKE. In some embodiments, a Designed Cytokine of the disclosure may be used combination with an interferon alpha polypeptide, including an interferon alpha 6, polypeptide having the sequence of MALPFALLMALVVLSCKSSCSLDCDLPQTHSLGHRRTMMLLAQMRRISLFSCLKDRHD FRFPQEEFDGNQFQKAEAISVLHEVIQQTFNLFSTKDSSVAWDERLLDKLYTELYQQLN DLEACVMQEVWVGGTPLMNEDSILA VRKYFQRITLYLTEKKY SPCA WEVVRAEIMR SF SSSRNLQERLRRKE. In some embodiments, a Designed Cytokine of the disclosure may be used combination with an interferon alpha polypeptide, including an interferon alpha 7, polypeptide having the sequence of MARSFSLLMVVLVLSYKSICSLGCDLPQTHSLRNRRALILLAQMGRISPFSCLKDRHEFR FPEEEFDGHQFQKTQAISVLHEMIQQTFNLFSTEDSSAAWEQSLLEKFSTELYQQLNDLE ACVIQEVGVEETPLMNEDFILA VRKYFQRITLYLMEKKYSPCAWEVVRAEIMRSFSFST NLKKGLRRKD. In some embodiments, a Designed Cytokine of the disclosure may be used combination with an interferon alpha polypeptide, including an interferon alpha 8, polypeptide having the sequence of MALTFYLLVALVVLSYKSFSSLGCDLPQTHSLGNRRALILLAQMRRISPFSCLKDRHDFE FPQEEFDDKQFQKAQAISVLHEMIQQTFNLFSTKDSSAALDETLLDEFYIELDQQLNDLE SCVMQEVGVIESPLMYEDSILA VRKYFQRITLYLTEKKYSSCA WEVVRAEIMRSFSLSIN LQKRLKSKE. In some embodiments, a Designed Cytokine of the disclosure may be used combination with an interferon alpha polypeptide, including an interferon alpha 10, polypeptide having the sequence of MALSFSLLMAVLVLSYKSICSLGCDLPQTHSLGNRRALILLGQMGRISPFSCLKDRHDFR IPQEEFDGNQFQKAQAISVLHEMIQQTFNLFSTEDSSAAWEQSLLEKFSTELYQQLNDLE ACVIQEVGVEETPLMNEDSILA VRKYFQRITLYLIERKYSPCAWEVVRAEIMRSLSFSTN LQKRLRRKD. In some embodiments, a Designed Cytokine of the disclosure may be used combination with an interferon alpha polypeptide, including an interferon alpha 14, polypeptide having the sequence of MALPFALMMALVVLSCKSSCSLGCNLSQTHSLNNRRTLMLMAQMRRISPFSCLKDRHD FEFPQEEFDGNQFQKAQAISVLHEMMQQTFNLFSTKNSSAAWDETLLEKFYIELFQQMN DLEACVIQEVGVEETPLMNEDSILAVKKYFQRITLYLMEKKYSPCAWEVVRAEIMRSLS FSTNLQKRLRRKD. In some embodiments, a Designed Cytokine of the disclosure may be used combination with an interferon alpha polypeptide, including an interferon alpha 17, polypeptide having the sequence of MALSFSLLMAVLVLSYKSICSLGCDLPQTHSLGNRRALILLAQMGRISPFSCLKDRHDFG LPQEEFDGNQFQKTQAISVLHEMIQQTFNLFSTEDSSAAWEQSLLEKFSTELYQQLNNLE ACVIQEVGMEETPLMNEDSILA VRKYFQRITLYLTEKKYSPCA WEVVRAEIMRSLSFST NLQKILRRKD. In some embodiments, a Designed Cytokine of the disclosure may be used combination with an interferon alpha polypeptide, including an interferon alpha 21, polypeptide having the sequence of

MALSFSLLMAVLVLSYKSICSLGCDLPQTHSLGNRRALILLAQMG
RISPFSCLKDRHDFGFPQEEFDGNQFQKAQAISVLHEMIQQTFNL
FSTKDSSATWEQSLLEKFSTELNQQLNDLEACVIQEVGVEETPLM
NVDSILAVKKYFQRITLYLTEKKYSPCAWEVVRAEIMRSFSLSKI
FQERLRRKE.

In some embodiments, a Designed Cytokine of the disclosure may be used combination with an interferon beta polypeptide, including an interferon beta polypeptide having the sequence of

MTNKCLLQIALLLCFSTTALSMSYNLLGFLQRSSNFQCQKLLWQL
NGRLEYCLKDRMNFDIPEEIKQLQQFQKEDAALTIYEMLQNIFAI
FRQDSSSTGWNETIVENLLANVYHQINHLKTVLEEKLEKEDFTRG
KLMSSLHLKRYYGRILHYLKAKEYSHCAWTIVRVEILRNFYFINR
LTGYLRN.

In some embodiments, a Designed Cytokine of the disclosure may be used combination with an interferon gamma polypeptide, including an interferon gamma polypeptide having the sequence of

MKYTSYILAFQLCIVLGSLGCYCQDPYVKEAENLKKYFNAGHSDV
ADNGTLFLGILKNWKEESDRKIMQSQIVSFYFKLFKNFKDDQSIQ
KSVETIKEDMNVKFFNSNKKKRDDFEKLTNYSVTDLNVQRKAIHE
LIQVMAELSPAAKTGKRKRSQMLFRGRRASQ.

In some embodiments, a Designed Cytokine of the disclosure may be used combination with an interferon omega polypeptide, including an interferon omega polypeptide having the sequence of

MALLFPLLAALVMTSYSPVGSLGCDLPQNHGLLSRNTLVLLHQMRRISP
FLCLKDRRDFRFPQEMVKGSQLQKAHVMSVLHEMLQQIFSLFHTERSSA
AWNMTLLDQLHTGLHQQLQHLETCLLQVVGEGESAGAISSPALTLRRYF
QGIRVYLKEKKYSDCAWEVVRMEIMKSLFLSTNMQERLRSKDRDLGSS.

### Exemplary Designed Cytokine Sequences

Exemplary Designed Cytokines of the disclosure include, but are not limited to, one or more of the polypeptides provided in any one of Tables 1-15. In some embodiments, Designed Cytokines of the disclosure comprise a polypeptide having at least 70%, 75%, 80%, 85%, 90%, 95%, 97%, 99%, or any percentage identity in between with a Designed Cytokine of the disclosure. In some embodiments, Designed Cytokines of the disclosure comprise a polypeptide having a sequence of one or more of helix H1, helix H4, helix H2, helix H3, of the IL-2/15 polypeptides of the disclosure, optionally, when the order of the helices is H1, H4, H2 and then H3 in the polypeptide from its amino terminus to its carboxy terminus. In some embodiments, IL-2/15 polypeptides of the disclosure comprise a polypeptide having a sequence of one or more of helix H1, helix H4, helix H2, helix H3, of the Designed Cytokines of the disclosure, when the order of the helices is H1, H4, H2 and then H3 in the polypeptide from its amino terminus to its carboxy terminus. In some embodiments, Designed Cytokines of the disclosure comprise a polypeptide having helix H1, helix H4, helix H2, helix H3 of the Designed Cytokines of the disclosure, in this order from its amino terminus to its carboxy terminus. In some embodiments, Designed Cytokines of the disclosure comprise a polypeptide having helix H1, helix H4, helix H2, helix H3 of the Designed Cytokines of the disclosure, in this order from its amino terminus to its carboxy terminus and, optionally, a loop connecting each helix having substantially the same topology and/or secondary structure as a loop within a Designed Cytokine of the disclosure.

TABLE 1

| Designed | SEQ | |
|---|---|---|
| Cytokine | ID | |
| No. | NO | Sequence |
| 1 | 1 | APTSSSTKKTQLQLEHLLLDLQMILNGINN |
| | | MNADPELVEFLNRWITFCQSLIKKGSNEDL |
| | | KHLQALEEELKPLEEVLNLAQSKNFHLRPR |
| | | DLISNINVIVLELKGS |
| 2 | 2 | APTSSSTKKTQLQLEHLLLDLQMILNGINN |
| | | GNADPELVEFLNRWITFCQSLIKKGSNEDL |
| | | KHLQALEEELKPLEEVLNLAQSKNFHLRPR |
| | | DLISNINVIVLELKGS |
| 3 | 3 | APTSSSTKKTQLQLEHLLLDLQMILNGINN |
| | | MNADPELVEFLNRWITFCQTAISTGKDNLK |
| | | HLQCLEEELKPLEEVLNLAQSKNFHLRPRD |
| | | LISNINVIVLELKGS |
| 4 | 4 | APTSSSTKKTQLQLEHLLLDLQMILNGINN |
| | | GNADPELVEFLNRWITFCQTAISTGKDNLK |
| | | HLQALEEELKPLEEVLNLAQSKNFHLRPRD |
| | | LISNINVIVLELKGS |
| 5 | 5 | APTSSSTKKTQLQLEHLLLDLQMILNALNK |
| | | LNIDSSIVEFLNRWITFCQTAISTGKDNLK |
| | | HLQALEEELKPLEEVLNLAQSKNFHLRPRD |
| | | LISNINVIVLELKGS |
| 6 | 6 | APTSSSTKKTQLQLEHLLLDLQMILNALNK |
| | | GNIDSSIVEFLNRWITFCQTAISTGKDNLK |
| | | HLQALEEELKPLEEVLNLAQSKNFHLRPRD |
| | | LISNINVIVLELKGS |
| 7 | 7 | APTSSSTKKTQLQLEHLLLDLQMILNGAEV |
| | | DPKIKEFLNRWITFCQSVIKANKDTREELK |
| | | HLQALEEELKPLEEVLNLAQSKNFHLRPRD |
| | | LISNINVIVLELKGS |
| 8 | 8 | APTSSSTKKTQLQLEHLLLDLQMILNALNK |
| | | LNINDHIVEFLNRWITFCQSATSDPDLLKH |
| | | LQALEEELKPLEEVLNLAQSKNFHLRPRDL |
| | | ISNINVIVLELKGS |
| 9 | 9 | APTSSSTKKTQLQLEHLLLDLQMILNGMNN |
| | | MNADPELVEFLNRWITFCESGDNTQEKLKC |
| | | LEEELKPLEEVLNLAQSKNFHLRPRDLISN |
| | | INVIVLELAGS |
| 10 | 10 | APTSSSTKKTQLQLEHLLLDLQMILNALKK |
| | | LNINSPMQEFLNRWITFAQSGSADLKHLQA |
| | | LEEELKPLEEVLNLAQSKNFHLRPRDLISN |
| | | INVIVLELKGS |
| 11 | 11 | APTSSSTKKTQLQLEHLLLDLQMILNALKK |
| | | GNINSPMQEFLNRWITFAQSGSADLKHLQA |
| | | LEEELKPLEEVLNLAQSKNFHLRPRDLISN |
| | | INVIVLELKGS |
| 12 | 12 | APTSSSTKKTQLQLEHLLLDLQMILNGMNN |
| | | MNADPELVEFLNRWITFAQKGDESALKHLQ |
| | | ALEEELKPLEEVLNLAQSKNFHLRPRDLIS |
| | | NINVIVLELKGS |
| 13 | 13 | APTSSSTKKTQLQLEHLLLDLQMILNGMNN |
| | | GNADPELVEFLNRWITFAQKGDESALKHLQ |
| | | ALEEELKPLEEVLNLAQSKNFHLRPRDLIS |
| | | NINVIVLELKGS |
| 14 | 14 | APTSSSTKKTQLQLEHLRLDLQMILNGMNN |
| | | MNADPELVEFLNRWITEAESSNATLKSLQA |
| | | LEEELKPLEEVLNLAQSKNFHLRPRDLISN |
| | | INVIVLELKGS |
| 15 | 15 | APTSSSTKKTQLQLEHLRLDLQMILNGMNN |
| | | GNADPELVEFLNRWITEAESSNATLKSLQA |
| | | LEEELKPLEEVLNLAQSKNFHLRPRDLISN |
| | | INVIVLELKGS |
| 16 | 16 | APTSSSTKKTQLQLEHLLLDLQMILNGMNN |
| | | MNADPELVEFLNRWITFAQDGSADLKHLQA |
| | | LEEELKPLEEVLNLAQSKNFHLRPRDLISN |
| | | INVIVLELKGS |
| 17 | 17 | APTSSSTKKTQLQLEHLLLDLQMILNGMNN |
| | | GNADPELVEFLNRWITFAQDGSADLKHLQA |
| | | LEEELKPLEEVLNLAQSKNFHLRPRDLISN |
| | | INVIVLELKGS |
| 18 | 18 | APTSSSTKKTQLQLEHLLLDLQMILNGINN |
| | | MNADPELVEFLNRWITFAQDGSADLKHLQA |
| | | LEEELKPLEEVLNLAQSKNFHLRPRDLISN |
| | | INVIVLELKGS |
| 19 | 19 | APTSSSTKKTQLQLEHLLLDLQMIANALNK |
| | | LGIESPFLNRWITFCQSNHEDAAKHLQALE |
| | | EELKPLEEVLNLAQSKNFHLRPRDLISNIN |
| | | VIVLELMGS |
| 20 | 20 | APTSSSTKKTQLQLEHLLLDLQMIANALNK |
| | | LGIESPFLNRWITFCQSVISASDDTREELK |
| | | HLQALEEELKPLEEVLNLAQSKNFHLRPRD |
| | | LISNINVIVLELKGS |
| 21 | 21 | APTSSSTKKTQLQLEHLLLDLQMILNALKK |
| | | LNINSPMQEFLNRWITFCESNTEDALKHLQ |
| | | ALEEELKPLEEVLNLAQSKNFHLRPRDLIS |
| | | NINVIVLELKGS |
| 22 | 22 | APTSSSTKKTQLQLEHLLLDLQMILNLAEV |
| | | DPTMKEFLNRWITFCESNSESALKHLQALE |
| | | EELKPLEEVLNLAQSKNFHLRPRDLISNIN |
| | | VIVLELKGS |
| 23 | 23 | APTSSSTKKTQLQLEHLLLDLQMILNFPVD |
| | | SEMVEFLNRWITFAQKGDESALKHLQALEE |
| | | ELKPLEEVLNLAQSKNFHLRPRDLISNINV |
| | | IVLELKGS |
| 24 | 24 | APTSSSTKKTQLQLEHLELDLQMILNLAEV |
| | | DPTMKEFLNRWITEAQSSNATLKSLQALEE |
| | | ELKPLEEVLNLAQSKNFHLRPRDLISNINV |
| | | IVLELKGS |
| 25 | 25 | APTSSSTKKTQLQLEHLLLDLQMILNFPVD |
| | | SEMVEFLNRWITFAEKGTEDANKELQALEE |
| | | ELKPLEEVLNLAQSKNFHLRPRDLISNINV |
| | | IVLELKGS |
| 26 | 26 | APTSSSTKKTQLQLEHLLLDLQMILNFPVD |
| | | SEMVEFLNRWITFAEKGTEDALKELQALEE |
| | | ELKPLEEVLNLAQSKNFHLRPRDLISNINV |
| | | IVLELKGS |
| 27 | 27 | APTSSSTKKTQLQLEHLLLDLQMILNGINN |
| | | MNADPELVEFLNRWITFCQSLIKKGSNEDL |
| | | KHLQALEERLKPLEEVLNRAQSKNFHLRPR |
| | | DLISNINVIVLELKGS |
| 28 | 28 | APTSSSTKKTQLQLEHLLLDLQMILNGAEV |
| | | DPKIKEFLNRWITFCQSVIKANKDTREELK |
| | | HLQALEERLKPLEEVLNRAQSKNFHLRPRD |
| | | LISNINVIVLELKGS |
| 29 | 29 | APTSSSTKKTQLQLEHLLLDLQMILNALNK |
| | | LNINDHIVEFLNRWITFCQSATSDPDLLKH |
| | | LQALEERLKPLEEVLNRAQSKNFHLRPRDL |
| | | ISNINVIVLELKGS |
| 30 | 30 | APTSSSTKKTQLQLEHLLLDLQMILNALKK |
| | | LNINSPMQEFLNRWITFCESNTEDALKHLQ |
| | | ALEERLKPLEEVLNRAQSKNFHLRPRDLIS |
| | | NINVIVLELKGS |
| 31 | 31 | APTSSSTKKTQLQLEHLLLDLQMILNFPVD |
| | | SEMVEFLNRWITFAEKGTEDALKELQALEE |
| | | RLKPLEEVLNRAQSKNFHLRPRDLISNINV |
| | | IVLELKGS |
| 32 | 32 | APTSSSTKKTQLQLEHLLLDLQMILNGINN |
| | | YKNPKLTRMLTFGGTATIVEFLNRWITACQ |
| | | READPEALQALEEELKPLEEVLNLAQSKNF |
| | | HLRPRDLISNINVIVLELMGS |
| 33 | 33 | APTSSSTKKTQLQAEHLLLDLQMILNGINN |
| | | YKNPKLTRMLTFGGTATIVEFLNRWITFCE |
| | | SGADEEAAKSLQALEEELKPLEEVLNLAQS |
| | | KNFHLRPRDLISNINVIVLELKGS |
| 34 | 34 | APTSSSTKKTQLQLEHLLLDLQMILNGINN |
| | | YKNPKLTEIDPKLVEFLNRWITFCQSAIKS |
| | | GSNTAKELQALEEELKPLEEVLNLAQSKNF |
| | | HLRPRDLISNINVIVLELAGS |
| 35 | 35 | APTSSSTKKTQLQLEHLLLDLQMILNGINN |
| | | YKNPKLTEIDPKLVEFLNRWITFCQTIING |
| | | GESATLKHLQALEEELKPLEEVLNLAQSKN |
| | | FHLRPRDLISNINVIVLELKGS |
| 36 | 36 | APTSSSTKKTQLQLEHLLLDLQMILNGINN |
| | | YKNPKLTEIDPKLVEFLNRWITFCQSIING |
| | | DESFSLKHLQALEEELKPLEEVLNLAQSKN |
| | | FHLRPRDLISNINVIVLELKGS |
| 37 | 37 | APTSSSTKKTQLQLEHLLLDLQMILNGINN |
| | | YKNPKLTKLDPSLVEFLNRWITFCQSATSD |
| | | PDLLKHLQALEEELKPLEEVLNLAQSKNFH |
| | | LRPRDLISNINVIVLELKGS |
| 38 | 38 | APTSSSTKKTQLQLEHLLLDLQMILNGINN |
| | | MNADPELVEFLNRWITFCQTAISTGKDNLK |
| | | HLQALEEELKPLEEVLNLAQSKNFHLRPRD |
| | | LISNINVIVLELKGS |

TABLE 2

| Designed | SEQ | | SEQ | | SEQ | | SEQ | |
|---|---|---|---|---|---|---|---|---|
| Cytokine | ID | | ID | | ID | | ID | |
| No. | NO | Helix 1 | NO | Helix 4 | NO | Helix 2 | NO | Helix 3 |
| 1 | 39 | SSTKKTQL | 52 | PELVEFLNR | 78 | EDLKHLQA | 98 | PRDLISNIN |
| | | QLEHLLLD | | WITFCQSLI | | LEEELKPLE | | VIVLELK |
| | | LQMILNGIN | | KK | | EVLNLA | | |
| | | NM | | | | | | |
| 2 | 40 | SSTKKTQL | 52 | PELVEFLNR | 78 | EDLKHLQA | 98 | PRDLISNIN |
| | | QLEHLLLD | | WITFCQSLI | | LEEELKPLE | | VIVLELK |
| | | LQMILNGIN | | KK | | EVLNLA | | |
| | | NG | | | | | | |
| 3 | 39 | SSTKKTQL | 53 | PELVEFLNR | 79 | LKHLQCLE | 98 | PRDLISNIN |
| | | QLEHLLLD | | WITFCQTAI | | EELKPLEEV | | VIVLELK |
| | | LQMILNGIN | | ST | | LNLA | | |
| | | NM | | | | | | |
| 4 | 40 | SSTKKTQL | 54 | PELVEFLNR | 80 | LKHLQALE | 98 | PRDLISNIN |
| | | QLEHLLLD | | WITFCQTAI | | EELKPLEEV | | VIVLELK |
| | | LQMILNGIN | | ST | | LNLA | | |
| | | NG | | | | | | |
| 5 | 41 | SSTKKTQL | 55 | SIVEFLNR | 80 | LKHLQALE | 98 | PRDLISNIN |
| | | QLEHLLLD | | WITFCQTAI | | EELKPLEEV | | VIVLELK |
| | | LQMILNAL | | ST | | LNLA | | |
| | | NK | | | | | | |
| 6 | 41 | SSTKKTQL | 56 | SIVEFLNR | 80 | LKHLQALE | 98 | PRDLISNIN |
| | | QLEHLLLD | | WITFCQTAI | | EELKPLEEV | | VIVLELK |
| | | LQMILNAL | | ST | | LNLA | | |
| | | NK | | | | | | |
| 7 | 42 | SSTKKTQL | 57 | PKIKEFLNR | 81 | TREELKHL | 98 | PRDLISNIN |
| | | QLEHLLLD | | WITFCQSVI | | QALEEELK | | VIVLELK |
| | | LQMILNG | | K | | PLEEVLNL | | |
| | | | | | | A | | |
| 8 | 41 | SSTKKTQL | 58 | HIVEFLNR | 82 | PDLLKHLQ | 98 | PRDLISNIN |
| | | QLEHLLLD | | WITFCQS | | ALEEELKPL | | VIVLELK |
| | | LQMILNAL | | | | EEVLNLA | | |
| | | NK | | | | | | |
| 9 | 43 | SSTKKTQL | 59 | PELVEFLNR | 83 | QEKLKCLE | 99 | PRDLISNIN |
| | | QLEHLLLD | | WITFCE | | EELKPLEEV | | VIVLELA |
| | | LQMILNGM | | | | LNLA | | |
| | | NNM | | | | | | |
| 10 | 44 | SSTKKTQL | 60 | MQEFLNR | 80 | LKHLQALE | 98 | PRDLISNIN |
| | | QLEHLLLD | | WITFAQ | | EELKPLEEV | | VIVLELK |
| | | LQMILNAL | | | | LNLA | | |
| | | KK | | | | | | |
| 11 | 44 | SSTKKTQL | 60 | MQEFLNR | 80 | LKHLQALE | 98 | PRDLISNIN |
| | | QLEHLLLD | | WITFAQ | | EELKPLEEV | | VIVLELK |
| | | LQMILNAL | | | | LNLA | | |
| | | KK | | | | | | |
| 12 | 43 | SSTKKTQL | 61 | PELVEFLNR | 84 | ESALKHLQ | 98 | PRDLISNIN |
| | | QLEHLLLD | | WITFAQK | | ALEEELKPL | | VIVLELK |
| | | LQMILNGM | | | | EEVLNLA | | |
| | | NNM | | | | | | |
| 13 | 45 | SSTKKTQL | 62 | PELVEFLNR | 85 | QALEEELK | 100 | DLISNINVI |
| | | QLEHLLLD | | WITFAQKG | | PLEEVLNL | | VLELK |
| | | LQMILNGM | | D | | A | | |
| | | NNG | | | | | | |
| 14 | 46 | SSTKKTQL | 63 | PELVEFLNR | 86 | KSLQALEE | 98 | PRDLISNIN |
| | | QLEHLRLD | | WITEAE | | ELKPLEEVL | | VIVLELK |
| | | LQMILNGM | | | | NLA | | |
| | | NNM | | | | | | |
| 15 | 47 | SSTKKTQL | 63 | PELVEFLNR | 86 | KSLQALEE | 98 | PRDLISNIN |
| | | QLEHLRLD | | WITEAE | | ELKPLEEVL | | VIVLELK |
| | | LQMILNGM | | | | NLA | | |
| | | NNG | | | | | | |
| 16 | 43 | SSTKKTQL | 64 | PELVEFLNR | 80 | LKHLQALE | 98 | PRDLISNIN |
| | | QLEHLLLD | | WITFAQ | | EELKPLEEV | | VIVLELK |
| | | LQMILNGM | | | | LNLA | | |
| | | NNM | | | | | | |
| 17 | 45 | SSTKKTQL | 64 | PELVEFLNR | 80 | LKHLQALE | 98 | PRDLISNIN |
| | | QLEHLLLD | | WITFAQ | | EELKPLEEV | | VIVLELK |
| | | LQMILNGM | | | | LNLA | | |
| | | NNG | | | | | | |
| 18 | 39 | SSTKKTQL | 64 | PELVEFLNR | 80 | LKHLQALE | 98 | PRDLISNIN |
| | | QLEHLLLD | | WITFAQ | | EELKPLEEV | | VIVLELK |
| | | LQMILNGIN | | | | LNLA | | |
| | | NM | | | | | | |
| 19 | 48 | SSTKKTQL | 65 | PFLNRWITF | 87 | AAKHLQAL | 101 | PRDLISNIN |
| | | QLEHLLLD | | CQS | | EEELKPLEE | | VIVLELM |
| | | LQMIANAL | | | | VLNLA | | |
| | | NKL | | | | | | |
| 20 | 48 | SSTKKTQL | 66 | PFLNRWITF | 81 | TREELKHL | 98 | PRDLISNIN |
| | | QLEHLLLD | | CQSVI | | QALEEELK | | VIVLELK |
| | | LQMIANAL | | | | PLEEVLNL | | |
| | | NKL | | | | A | | |
| 21 | 44 | SSTKKTQL | 67 | MQEFLNR | 80 | LKHLQALE | 98 | PRDLISNIN |
| | | QLEHLLLD | | WITFCES | | EELKPLEEV | | VIVLELK |
| | | LQMILNAL | | | | LNLA | | |
| | | KK | | | | | | |
| 22 | 49 | SSTKKTQL | 68 | PTMKEFLN | 80 | LKHLQALE | 98 | PRDLISNIN |
| | | QLEHLLLD | | RWITFCES | | EELKPLEEV | | VIVLELK |
| | | LQMILNL | | | | LNLA | | |
| 23 | 50 | SSTKKTQL | 69 | SEMVEFLN | 84 | ESALKHLQ | 98 | PRDLISNIN |
| | | QLEHLLLD | | RWITFAQK | | ALEEELKPL | | VIVLELK |
| | | LQMILN | | | | EEVLNLA | | |
| 24 | 51 | SSTKKTQL | 70 | PTMKEFLN | 86 | KSLQALEE | 98 | PRDLISNIN |
| | | QLEHLELD | | RWITEAQ | | ELKPLEEVL | | VIVLELK |
| | | LQMILNL | | | | NLA | | |
| 25 | 50 | SSTKKTQL | 71 | SEMVEFLN | 88 | EDANKELQ | 98 | PRDLISNIN |
| | | QLEHLLLD | | RWITFAEK | | ALEEELKPL | | VIVLELK |
| | | LQMILN | | | | EEVLNLA | | |
| 26 | 50 | SSTKKTQL | 71 | SEMVEFLN | 89 | EDALKELQ | 98 | PRDLISNIN |
| | | QLEHLLLD | | RWITFAEK | | ALEEELKPL | | VIVLELK |
| | | LQMILN | | | | EEVLNLA | | |
| 27 | 39 | SSTKKTQL | 52 | PELVEFLNR | 90 | EDLKHLQA | 98 | PRDLISNIN |
| | | QLEHLLLD | | WITFCQSLI | | LEERLKPLE | | VIVLELK |
| | | LQMILNGIN | | KK | | EVLNRA | | |
| | | NM | | | | | | |
| 28 | 42 | SSTKKTQL | 57 | PKIKEFLNR | 91 | TREELKHL | 98 | PRDLISNIN |
| | | QLEHLLLD | | WITFCQSVI | | QALEERLK | | VIVLELK |
| | | LQMILNG | | K | | PLEEVLNR | | |
| | | | | | | A | | |
| 29 | 41 | SSTKKTQL | 58 | HIVEFLNR | 92 | PDLLKHLQ | 98 | PRDLISNIN |
| | | QLEHLLLD | | WITFCQS | | ALEERLKP | | VIVLELK |
| | | LQMILNAL | | | | LEEVLNRA | | |
| | | NK | | | | | | |
| 30 | 44 | SSTKKTQL | 67 | MQEFLNR | 93 | LKHLQALE | 98 | PRDLISNIN |
| | | QLEHLLLD | | WITFCES | | ERLKPLEE | | VIVLELK |
| | | LQMILNAL | | | | VLNRA | | |
| | | KK | | | | | | |
| 31 | 50 | SSTKKTQL | 71 | SEMVEFLN | 94 | EDALKELQ | 98 | PRDLISNIN |
| | | QLEHLLLD | | RWITFAEK | | ALEERLKP | | VIVLELK |
| | | LQMILN | | | | LEEVLNRA | | |
| 32 | | | 72 | IVEFLNRWI | 95 | PEALQALE | 101 | PRDLISNIN |
| | | | | TAC | | EELKPLEEV | | VIVLELM |
| | | | | | | LNLA | | |
| 33 | | | 73 | IVEFLNRWI | | EEAAKSLQ | 98 | PRDLISNIN |
| | | | | | | ALEEELKPL | | |
| | | | | TFCE | 96 | EEVLNLA | | VIVLELK |
| 34 | | | 74 | PKLVEFLN | 97 | AKELQALE | 99 | PRDLISNIN |
| | | | | RWITFCQS | | EELKPLEEV | | VIVLELA |
| | | | | AIKS | | LNLA | | |
| 35 | | | 75 | PKLVEFLN | 80 | LKHLQALE | 98 | PRDLISNIN |
| | | | | RWITFCQTI | | EELKPLEEV | | VIVLELK |
| | | | | I | | LNLA | | |
| 36 | | | 76 | PKLVEFLN | 80 | LKHLQALE | 98 | PRDLISNIN |
| | | | | RWITFCQSI | | EELKPLEEV | | VIVLELK |
| | | | | I | | LNLA | | |
| 37 | | | 77 | PSLVEFLNR | 82 | PDLLKHLQ | 98 | PRDLISNIN |
| | | | | WITFCQS | | ALEEELKPL | | VIVLELK |
| | | | | | | EEVLNLA | | |
| 38 | 39 | SSTKKTQL | 54 | PELVEFLNR | 80 | LKHLQALE | 98 | PRDLISNIN |
| | | QLEHLLLD | | WITFCQTAI | | EELKPLEEV | | VIVLELK |
| | | LQMILNGIN | | ST | | LNLA | | |
| | | NM | | | | | | |

TABLE 3

| Designed | SEQ | | SEQ | | SEQ | |
|---|---|---|---|---|---|---|
| Cytokine | ID | Loop | ID | | ID | |
| No. | NO | 1 | NO | Loop 2 | NO | Loop 3 |
| 1 | 102 | NAD | 112 | GSN | 132 | QSKNFHLR |
| 2 | 102 | NAD | 112 | GSN | 132 | QSKNFHLR |
| 3 | 102 | NAD | 113 | GKDN | 132 | QSKNFHLR |
| 4 | 102 | NAD | 113 | GKDN | 132 | QSKNFHLR |
| 5 | 103 | LNIDS | 113 | GKDN | 132 | QSKNFHLR |
| 6 | 104 | GNIDS | 113 | GKDN | 132 | QSKNFHLR |
| 7 | 105 | AEVD | 114 | ANKD | 132 | QSKNFHLR |
| 8 | 106 | LNIND | 115 | ATSD | 132 | QSKNFHLR |
| 9 | 102 | NAD | 116 | SGDNT | 132 | QSKNFHLR |
| 10 | 108 | LNINSP | 117 | SGSAD | 132 | QSKNFHLR |
| 11 | 109 | GNINSP | 117 | SGSAD | 132 | QSKNFHLR |
| 12 | 102 | NAD | 118 | GD | 132 | QSKNFHLR |
| 13 | 102 | NAD | 119 | ESALKHL | 132 | QSKNFHLR |
| 14 | 102 | NAD | 120 | SSNATL | 132 | QSKNFHLR |
| 15 | 102 | NAD | 120 | SSNATL | 132 | QSKNFHLR |
| 16 | 102 | NAD | 121 | DGSAD | 132 | QSKNFHLR |
| 17 | 102 | NAD | 121 | DGSAD | 132 | QSKNFHLR |
| 18 | 102 | NAD | 121 | DGSAD | 132 | QSKNFHLR |
| 19 | 107 | GIES | 122 | NHED | 132 | QSKNFHLR |
| 20 | 107 | GIES | 123 | SASDD | 132 | QSKNFHLR |
| 21 | 108 | LNINSP | 124 | NTEDA | 132 | QSKNFHLR |
| 22 | 105 | AEVD | 125 | NSESA | 132 | QSKNFHLR |
| 23 | 110 | FPVD | 118 | GD | 132 | QSKNFHLR |
| 24 | 105 | AEVD | 120 | SSNATL | 132 | QSKNFHLR |
| 25 | 110 | FPVD | 126 | GT | 132 | QSKNFHLR |
| 26 | 110 | FPVD | 126 | GT | 132 | QSKNFHLR |
| 27 | 102 | NAD | 112 | GSN | 132 | QSKNFHLR |
| 28 | 105 | AEVD | 114 | ANKD | 132 | QSKNFHLR |
| 29 | 106 | LNIND | 115 | ATSD | 132 | QSKNFHLR |
| 30 | 108 | LNINSP | 124 | NTEDA | 132 | QSKNFHLR |
| 31 | 110 | FPVD | 126 | GT | 132 | QSKNFHLR |
| 32 | 111 | FGGTAT | 127 | QREAD | 132 | QSKNFHLR |
| 33 | 111 | FGGTAT | 128 | SGAD | 132 | QSKNFHLR |
| 34 | | | 129 | GSNT | 132 | QSKNFHLR |
| 35 | | | 130 | NGGESAT | 132 | QSKNFHLR |
| 36 | | | 131 | NGDESFS | 132 | QSKNFHLR |
| 37 | | | 115 | ATSD | 132 | QSKNFHLR |
| 38 | 102 | NAD | 113 | GKDN | 132 | QSKNFHLR |

TABLE 4

| | | | Sequence (Designed Cytokines of the disclosure do |
|---|---|---|---|
| | | | not comprise or consist of a sequence requiring |
| Designed | SEQ | | the bolded and underlined amino acids below, |
| Cytokine | ID | | i.e., the bolded and underlined amino |
| No. | NO | Features | acids are optional). |
| 39 | 150 | M28I_K56S_C65A | APTSSSTKKTQLQLEHLLLDLQMILNGINNMNADPELVEFL |
| | | | NRWITFCQSLIKKGSNEDLKHLQALEEELKPLEEVLNLAQS |
| | | | KNFHLRPRDLISNINVIVLELKGSGSGGSGNWSHPQFEK |
| 40 | 151 | M28I_K56S_C65A_ | APTSSSTKKTQLQLEHLLLDLQMILNGINNGNADPELVEFL |
| | | M31G | NRWITFCQSLIKKGSNEDLKHLQALEEELKPLEEVLNLAQS |
| | | | KNFHLRPRDLISNINVIVLELKGSGSGGSGNWSHPQFEK |
| 41 | 152 | M28I_T53S_C64A | APTSSSTKKTQLQLEHLLLDLQMILNGINNMNADPELVEFL |
| | | | NRWITFCQTAISTGKDNLKHLQCLEEELKPLEEVLNLAQSK |
| | | | NFHLRPRDLISNINVIVLELKGSGSGGSGNWSHPQFEK |
| 42 | 153 | M28I_T53S_C64A_ | APTSSSTKKTQLQLEHLLLDLQMILNGINNGNADPELVEFL |
| | | M31G | NRWITFCQTAISTGKDNLKHLQALEEELKPLEEVLNLAQSK |
| | | | NFHLRPRDLISNINVIVLELKGSGSGGSGNWSHPQFEK |
| 43 | 154 | T53S_C64A | APTSSSTKKTQLQLEHLLLDLQMILNALNKLNIDSSIVEFL |
| | | | NRWITFCQTAISTGKDNLKHLQALEEELKPLEEVLNLAQSK |
| | | | NFHLRPRDLISNINVIVLELKGSGSGGSGNWSHPQFEK |
| 44 | 155 | T53S_C64AL31G | APTSSSTKKTQLQLEHLLLDLQMILNALNKGNIDSSIVEFL |
| | | | NRWITFCQTAISTGKDNLKHLQALEEELKPLEEVLNLAQSK |
| | | | NFHLRPRDLISNINVIVLELKGSGSGGSGNWSHPQFEK |
| 45 | 156 | L27G_C64A | APTSSSTKKTQLQLEHLLLDLQMILNGAEVDPKIKEFLNRW |
| | | | ITFCQSVIKANKDTREELKHLQALEEELKPLEEVLNLAQSK |
| | | | NFHLRPRDLISNINVIVLELKGSGSGGSGNWSHPQFEK |
| 46 | 157 | C63A | APTSSSTKKTQLQLEHLLLDLQMILNALNKLNINDHIVEFL |
| | | | NRWITFCQSATSDPDLLKHLQALEEELKPLEEVLNLAQSKN |
| | | | FHLRPRDLISNINVIVLELKGSGSGGSGNWSHPQFEK |
| 47 | 158 | C60A | APTSSSTKKTQLQLEHLLLDLQMILNGMNNMNADPELVEFL |
| | | | NRWITFCESGDNTQEKLKCLEEELKPLEEVLNLAQSKNFHL |
| | | | RPRDLISNINVIVLELAGSGSGGSGNWSHPQFEK |
| 48 | 159 | D50S_R56K_C60A | APTSSSTKKTQLQLEHLLLDLQMILNALKKLNINSPMQEFL |
| | | | NRWITFAQSGSADLKHLQALEEELKPLEEVLNLAQSKNFHL |
| | | | RPRDLISNINVIVLELKGSGSGGSGNWSHPQFEK |
| 49 | 160 | D50S_R56K_C60A_ | APTSSSTKKTQLQLEHLLLDLQMILNALKKGNINSPMQEFL |
| | | L31G | NRWITFAQSGSADLKHLQALEEELKPLEEVLNLAQSKNFHL |
| | | | RPRDLISNINVIVLELKGSGSGGSGNWSHPQFEK |
| 50 | 161 | C61A | APTSSSTKKTQLQLEHLLLDLQMILNGMNNMNADPELVEFL |
| | | | NRWITFAQKGDESALKHLQALEEELKPLEEVLNLAQSKNFH |
| | | | LRPRDLISNINVIVLELKGSGSGGSGNWSHPQFEK |
| 51 | 162 | C61A_M31G | APTSSSTKKTQLQLEHLLLDLQMILNGMNNGNADPELVEFL |
| | | | NRWITFAQKGDESALKHLQALEEELKPLEEVLNLAQSKNFH |
| | | | LRPRDLISNINVIVLELKGSGSGGSGNWSHPQFEK |
| 52 | 163 | C60A | APTSSSTKKTQLQLEHLRLDLQMILNGMNNMNADPELVEFL |
| | | | NRWITEAESSNATLKSLQALEEELKPLEEVLNLAQSKNFHL |
| | | | RPRDLISNINVIVLELKGSGSGGSGNWSHPQFEK |
| 53 | 164 | C60A_M31G | APTSSSTKKTQLQLEHLRLDLQMILNGMNNGNADPELVEFL |
| | | | NRWITEAESSNATLKSLQALEEELKPLEEVLNLAQSKNFHL |
| | | | RPRDLISNINVIVLELKGSGSGGSGNWSHPQFEK |
| 54 | 165 | R56K_C60A | APTSSSTKKTQLQLEHLLLDLQMILNGMNNMNADPELVEFL |
| | | | NRWITFAQDGSADLKHLQALEEELKPLEEVLNLAQSKNFHL |
| | | | RPRDLISNINVIVLELKGSGSGGSGNWSHPQFEK |
| 55 | 166 | R56K_C60A_M31G | APTSSSTKKTQLQLEHLLLDLQMILNGMNNGNADPELVEFL |
| | | | NRWITFAQDGSADLKHLQALEEELKPLEEVLNLAQSKNFHL |
| | | | RPRDLISNINVIVLELKGSGSGGSGNWSHPQFEK |
| 56 | 167 | R56K_C60A_M28I | APTSSSTKKTQLQLEHLLLDLQMILNGINNMNADPELVEFL |
| | | | NRWITFAQDGSADLKHLQALEEELKPLEEVLNLAQSKNFHL |
| | | | RPRDLISNINVIVLELKGSGSGGSGNWSHPQFEK |
| 57 | 168 | C58A | APTSSSTKKTQLQLEHLLLDLQMIANALNKLGIESPFLNRW |
| | | | ITFCQSNHEDAAKHLQALEEELKPLEEVLNLAQSKNFHLRP |
| | | | RDLISNINVIVLELMGSGSGGSGNWSHPQFEK |
| 58 | 169 | N50S_C64A | APTSSSTKKTQLQLEHLLLDLQMIANALNKLGIESPFLNRW |
| | | | ITFCQSVISASDDTREELKHLQALEEELKPLEEVLNLAQSK |
| | | | NFHLRPRDLISNINVIVLELKGSGSGGSGNWSHPQFEK |
| 59 | 170 | C61A | APTSSSTKKTQLQLEHLLLDLQMILNALKKLNINSPMQEFL |
| | | | NRWITFCESNTEDALKHLQALEEELKPLEEVLNLAQSKNFH |
| | | | LRPRDLISNINVIVLELKGSGSGGSGNWSHPQFEK |
| 60 | 171 | C58A | APTSSSTKKTQLQLEHLLLDLQMILNLAEVDPTMKEFLNRW |
| | | | ITFCESNSESALKHLQALEEELKPLEEVLNLAQSKNFHLRP |
| | | | RDLISNINVIVLELKGSGSGGSGNWSHPQFEK |
| 61 | 172 | C57A | APTSSSTKKTQLQLEHLLLDLQMILNFPVDSEMVEFLNRWI |
| | | | TFAQKGDESALKHLQALEEELKPLEEVLNLAQSKNFHLRPR |
| | | | DLISNINVIVLELKGSGSGGSGNWSHPQFEK |
| 62 | 173 | C57A | APTSSSTKKTQLQLEHLELDLQMILNLAEVDPTMKEFLNRW |
| | | | ITEAQSSNATLKSLQALEEELKPLEEVLNLAQSKNFHLRPR |
| | | | DLISNINVIVLELKGSGSGGSGNWSHPQFEK |
| 63 | 174 | C57A | APTSSSTKKTQLQLEHLLLDLQMILNFPVDSEMVEFLNRWI |
| | | | TFAEKGTEDANKELQALEEELKPLEEVLNLAQSKNFHLRPR |
| | | | DLISNINVIVLELKGSGSGGSGNWSHPQFEK |
| 64 | 175 | N52L_C57A | APTSSSTKKTQLQLEHLLLDLQMILNFPVDSEMVEFLNRWI |
| | | | TFAEKGTEDALKELQALEEELKPLEEVLNLAQSKNFHLRPR |
| | | | DLISNINVIVLELKGSGSGGSGNWSHPQFEK |
| 65 | 176 | M28I_K56S_C65A_ | APTSSSTKKTQLQLEHLLLDLQMILNGINNMNADPELVEFL |
| | | E69R_L79R | NRWITFCQSLIKKGSNEDLKHLQALEERLKPLEEVLNRAQS |
| | | | KNFHLRPRDLISNINVIVLELKGSGSGGSGNWSHPQFEK |
| 66 | 177 | L27G_C64A_E68R_ | APTSSSTKKTQLQLEHLLLDLQMILNGAEVDPKIKEFLNRW |
| | | L78R | ITFCQSVIKANKDTREELKHLQALEERLKPLEEVLNRAQSK |
| | | | NFHLRPRDLISNINVIVLELKGSGSGGSGNWSHPQFEK |
| 67 | 178 | C63A_E67R_L77R | APTSSSTKKTQLQLEHLLLDLQMILNALNKLNINDHIVEFL |
| | | | NRWITFCQSATSDPDLLKHLQALEERLKPLEEVLNRAQSKN |
| | | | FHLRPRDLISNINVIVLELKGSGSGGSGNWSHPQFEK |
| 68 | 179 | C61A_E65RL75R | APTSSSTKKTQLQLEHLLLDLQMILNALKKLNINSPMQEFL |
| | | | NRWITFCESNTEDALKHLQALEERLKPLEEVLNRAQSKNFH |
| | | | LRPRDLISNINVIVLELKGSGSGGSGNWSHPQFEK |
| 69 | 180 | N52L_C57A_E61R_ | APTSSSTKKTQLQLEHLLLDLQMILNFPVDSEMVEFLNRWI |
| | | L71R | TFAEKGTEDALKELQALEERLKPLEEVLNRAQSKNFHLRPR |
| | | | DLISNINVIVLELKGSGSGGSGNWSHPQFEK |
| 70 | 181 | K43G_C70A | APTSSSTKKTQLQLEHLLLDLQMILNGINNYKNPKLTRMLT |
| | | | FGGTATIVEFLNRWITACQREADPEALQALEEELKPLEEVL |
| | | | NLAQSKNFHLRPRDLISNINVIVLELMGSGSGGSGNWSHPQ |
| | | | FEK |
| 71 | 182 | K43G_C73A | APTSSSTKKTQLQAEHLLLDLQMILNGINNYKNPKLTRMLT |
| | | | FGGTATIVEFLNRWITFCESGADEEAAKSLQALEEELKPLE |
| | | | EVLNLAQSKNFHLRPRDLISNINVIVLELKGSGSGGSGNWS |
| | | | HPQFEK |
| 72 | 183 | C70A | APTSSSTKKTQLQLEHLLLDLQMILNGINNYKNPKLTEIDP |
| | | | KLVEFLNRWITFCQSAIKSGSNTAKELQALEEELKPLEEVL |
| | | | NLAQSKNFHLRPRDLISNINVIVLELAGSGSGGSGNWSHPQ |
| | | | FEK |
| 73 | 184 | C71A | APTSSSTKKTQLQLEHLLLDLQMILNGINNYKNPKLTEIDP |
| | | | KLVEFLNRWITFCQTIINGGESATLKHLQALEEELKPLEEV |
| | | | LNLAQSKNFHLRPRDLISNINVIVLELKGSGSGGSGNWSHP |
| | | | QFEK |
| 74 | 185 | C71A | APTSSSTKKTQLQLEHLLLDLQMILNGINNYKNPKLTEIDP |
| | | | KLVEFLNRWITFCQSIINGDESFSLKHLQALEEELKPLEEV |
| | | | LNLAQSKNFHLRPRDLISNINVIVLELKGSGSGGSGNWSHP |
| | | | QFEK |
| 75 | 186 | C69A | APTSSSTKKTQLQLEHLLLDLQMILNGINNYKNPKLTKLDP |
| | | | SLVEFLNRWITFCQSATSDPDLLKHLQALEEELKPLEEVLN |
| | | | LAQSKNFHLRPRDLISNINVIVLELKGSGSGGSGNWSHPQF |
| | | | EK |
| 76 | 187 | M28I_T53S_C64A | APTSSSTKKTQLQLEHLLLDLQMILNGINNMNADPELVEFL |
| | | | NRWITFCQTAISTGKDNLKHLQALEEELKPLEEVLNLAQSK |
| | | | NFHLRPRDLISNINVIVLELKGSGSGGSGNWSHPQFEK |

TABLE 5
(Variants of IL-2 Polypeptide 39)

| Designed | | | Sequence (Designed Cytokines of the disclosure do not comprise or consist |
|---|---|---|---|
| Cytokine | SEQ | | of a sequence requiring the bolded and underlined amino acids below, |
| No. | ID NO | Features | i.e., the bolded and underlined amino acids are optional). |
| 77 | 188 | C48A | APTSSSTKKTQLQLEHLLLDLQMILNGINNMNADPELVEFLNRWITFAQSLIKKGSNEDLKHLQAL |
| | | | EEELKPLEEVLNLAQSKNFHLRPRDLISNINVIVLELKGSGSGGSGNWSHPQFEK |
| 78 | 189 | C48I | APTSSSTKKTQLQLEHLLLDLQMILNGINNMNADPELVEFLNRWITFIQSLIKKGSNEDLKHLQAL |
| | | | EEELKPLEEVLNLAQSKNFHLRPRDLISNINVIVLELKGSGSGGSGNWSHPQFEK |
| 79 | 190 | I28M_C48A | APTSSSTKKTQLQLEHLLLDLQMILNGMNNMNADPELVEFLNRWITFAQSLIKKGSNEDLKHLQAL |
| | | | EEELKPLEEVLNLAQSKNFHLRPRDLISNINVIVLELKGSGSGGSGNWSHPQFEK |
| 80 | 191 | A33G_C48A | APTSSSTKKTQLQLEHLLLDLQMILNGINNMNGDPELVEFLNRWITFAQSLIKKGSNEDLKHLQAL |
| | | | EEELKPLEEVLNLAQSKNFHLRPRDLISNINVIVLELKGSGSGGSGNWSHPQFEK |
| 81 | 192 | F40Q_C48A | APTSSSTKKTQLQLEHLLLDLQMILNGINNMNADPELVEQLNRWITFAQSLIKKGSNEDLKHLQAL |
| | | | EEELKPLEEVLNLAQSKNFHLRPRDLISNINVIVLELKGSGSGGSGNWSHPQFEK |
| 82 | 193 | F40K_C48A | APTSSSTKKTQLQLEHLLLDLQMILNGINNMNADPELVEKLNRWITFAQSLIKKGSNEDLKHLQAL |
| | | | EEELKPLEEVLNLAQSKNFHLRPRDLISNINVIVLELKGSGSGGSGNWSHPQFEK |
| 83 | 194 | C48A_C65(N-linked | APTSSSTKKTQLQLEHLLLDLQMILNGINNMNADPELVEFLNRWITFAQSLIKKGSNEDLKHLQNL |
| | | glycosylation site) | SEELKPLEEVLNLAQSKNFHLRPRDLISNINVIVLELKGSGSGGSGNWSHPQFEK |
| 84 | 195 | A33G_F40K_C48A | APTSSSTKKTQLQLEHLLLDLQMILNGINNMNGDPELVEKLNRWITFAQSLIKKGSNEDLKHLQAL |
| | | | EEELKPLEEVLNLAQSKNFHLRPRDLISNINVIVLELKGSGSGGSGNWSHPQFEK |
| 85 | 196 | 51I_GSGSD_C48A | APTSSSTKKTQLQLEHLLLDLQMILNGINNMNADPELVEFLNRWITFAQSIIKKGGSGSDLKHLQA |
| | | | LEEELKPLEEVLNLAQSKNFHLRPRDLISNINVIVLELKGSGSGGSGNWSHPQFEK |
| 86 | 197 | 51I_GSGSD_A33G_C48A | APTSSSTKKTQLQLEHLLLDLQMILNGINNMNGDPELVEFLNRWITFAQSIIKKGGSGSDLKHLQA |
| | | | LEEELKPLEEVLNLAQSKNFHLRPRDLISNINVIVLELKGSGSGGSGNWSHPQFEK |
| 87 | 198 | 51I_GSGSD_A33G_F40K_C48A | APTSSSTKKTQLQLEHLLLDLQMILNGINNMNGDPELVEKLNRWITFAQSIIKKGGSGSDLKHLQA |
| | | | LEEELKPLEEVLNLAQSKNFHLRPRDLISNINVIVLELKGSGSGGSGNWSHPQFEK |
| 88 | 199 | 51ISTG_GSGSD_A33G_C48A | APTSSSTKKTQLQLEHLLLDLQMILNGINNMNGDPELVEFLNRWITFAQSISTGGSGSDLKHLQAL |
| | | | EEELKPLEEVLNLAQSKNFHLRPRDLISNINVIVLELKGSGSGGSGNWSHPQFEK |
| 89 | 200 | GAGD_A33G_C48A | APTSSSTKKTQLQLEHLLLDLQMILNGINNMNGDPELVEFLNRWITFAQSLIKKGGAGDLKHLQAL |
| | | | EEELKPLEEVLNLAQSKNFHLRPRDLISNINVIVLELKGSGSGGSGNWSHPQFEK |
| 90 | 201 | KGED_A33G_C48A | APTSSSTKKTQLQLEHLLLDLQMILNGINNMNGDPELVEFLNRWITFAQSLIKKGKGEDLKHLQAL |
| | | | EEELKPLEEVLNLAQSKNFHLRPRDLISNINVIVLELKGSGSGGSGNWSHPQFEK |
| 91 | 202 | 51I_GSGSD_A33G_F40K_C48A_ | APTSSSTKKTQLQLEHLLLDLQMILNGINNMNGDPELVEKLNRWITFAQSIIKKGGSGSDLKHLQN |
| | | C65(N-linked glycosylation | LSEELKPLEEVLNLAQSKNFHLRPRDLISNINVIVLELKGSGSGGSGNWSHPQFEK |
| | | site) | |
| 92 | 203 | L14C | APTSSSTKKTQLQCEHLLLDLQMILNGINNMNADPELVEFLNRWITFCQSLIKKGSNEDLKHLQAL |
| | | | EEELKPLEEVLNLAQSKNFHLRPRDLISNINVIVLELKGSGSGGSGNWSHPQFEK |
| 93 | 204 | L18C | APTSSSTKKTQLQLEHLCLDLQMILNGINNMNADPELVEFLNRWITFCQSLIKKGSNEDLKHLQAL |
| | | | EEELKPLEEVLNLAQSKNFHLRPRDLISNINVIVLELKGSGSGGSGNWSHPQFEK |
| 94 | 205 | C48A_E69(N-linked | APTSSSTKKTQLQLEHLLLDLQMILNGINNMNADPELVEFLNRWITFAQSLIKKGSNEDLKHLQAL |
| | | glycosylation site) | EENLSPLEEVLNLAQSKNFHLRPRDLISNINVIVLELKGSGSGGSGNWSHPQFEK |
| 95 | 206 | 51ISTG_GSGSD_M28_C48A | APTSSSTKKTQLQLEHLLLDLQMILNGMNNMNADPELVEFLNRWITFAQSISTGGSGSDLKHLQAL |
| | | | EEELKPLEEVLNLAQSKNFHLRPRDLISNINVIVLELKGSGSGGSGNWSHPQFEK |
| 96 | 207 | 51ISTG_GSGSD_M28_G31_C48A | APTSSSTKKTQLQLEHLLLDLQMILNGMNNGNADPELVEFLNRWITFAQSISTGGSGSDLKHLQAL |
| | | | EEELKPLEEVLNLAQSKNFHLRPRDLISNINVIVLELKGSGSGGSGNWSHPQFEK |
| 97 | 208 | KGED_M28_C48A | APTSSSTKKTQLQLEHLLLDLQMILNGMNNMNADPELVEFLNRWITFAQSLIKKGKGEDLKHLQAL |
| | | | EEELKPLEEVLNLAQSKNFHLRPRDLISNINVIVLELKGSGSGGSGNWSHPQFEK |
| 98 | 209 | 51I_GSGSD_L18C_A33G_F40K | APTSSSTKKTQLQLEHLCLDLQMILNGINNMNGDPELVEKLNRWITFCQSIIKKGGSGSDLKHLQA |
| | | | LEEELKPLEEVLNLAQSKNFHLRPRDLISNINVIVLELKGSGSGGSGNWSHPQFEK |
| 99 | 210 | 51I_GSGSD_L18C_A33G_C65 | APTSSSTKKTQLQLEHLCLDLQMILNGINNMNGDPELVEFLNRWITFCQSIIKKGGSGSDLKHLQN |
| | | (N-linked glycosylation site) | LSEELKPLEEVLNLAQSKNFHLRPRDLISNINVIVLELKGSGSGGSGNWSHPQFEK |
| 100 | 211 | 51I_GSGSD_L18C_A33G_F40K_ | APTSSSTKKTQLQLEHLCLDLQMILNGINNMNGDPELVEKLNRWITFCQSIIKKGGSGSDLKHLQN |
| | | C65(N-linked glycosylation | LSEELKPLEEVLNLAQSKNFHLRPRDLISNINVIVLELKGSGSGGSGNWSHPQFEK |
| | | site) | |
| 101 | 212 | [W44I] | APTSSSTKKTQLQLEHLLLDLQMILNGINNMNADPELVEFLNRIITFCQSLIKKGSNEDLKHLQAL |
| | | | EEELKPLEEVLNLAQSKNFHLRPRDLISNINVIVLELKGSGSGGSGNWSHPQFEK |
| 102 | 213 | [F44L, W47L] | APTSSSTKKTQLQLEHLLLDLQMILNGINNMNADPELVEFLNRLITLCQSLIKKGSNEDLKHLQAL |
| | | | EEELKPLEEVLNLAQSKNFHLRPRDLISNINVIVLELKGSGSGGSGNWSHPQFEK |
| 103 | 214 | [F40K] | APTSSSTKKTQLQLEHLLLDLQMILNGINNMNADPELVEKLNRWITFCQSLIKKGSNEDLKHLQAL |
| | | | EEELKPLEEVLNLAQSKNFHLRPRDLISNINVIVLELKGSGSGGSGNWSHPQFEK |
| 104 | 215 | [F47E] | APTSSSTKKTQLQLEHLLLDLQMILNGINNMNADPELVEFLNRWITECQSLIKKGSNEDLKHLQAL |
| | | | EEELKPLEEVLNLAQSKNFHLRPRDLISNINVIVLELKGSGSGGSGNWSHPQFEK |
| 105 | 216 | [C65K] | APTSSSTKKTQLQLEHLLLDLQMILNGINNMNADPELVEFLNRWITFCQSLIKKGSNEDLKHLQKL |
| | | | EEELKPLEEVLNLAQSKNFHLRPRDLISNINVIVLELKGSGSGGSGNWSHPQFEK |
| 106 | 217 | [C65K, E69A] | APTSSSTKKTQLQLEHLLLDLQMILNGINNMNADPELVEFLNRWITFCQSLIKKGSNEDLKHLQKL |
| | | | EEALKPLEEVLNLAQSKNFHLRPRDLISNINVIVLELKGSGSGGSGNWSHPQFEK |
| 107 | 218 | [C65K, E69A, V76K] | APTSSSTKKTQLQLEHLLLDLQMILNGINNMNADPELVEFLNRWITFCQSLIKKGSNEDLKHLQKL |
| | | | EEALKPLEEKLNLAQSKNFHLRPRDLISNINVIVLELKGSGSGGSGNWSHPQFEK |
| 108 | 219 | [51-LIKK-54 to IIST] | APTSSSTKKTQLQLEHLLLDLQMILNGINNMNADPELVEFLNRWITFCQSIISTGSNEDLKHLQAL |
| | | | EEELKPLEEVLNLAQSKNFHLRPRDLISNINVIVLELKGSGSGGSGNWSHPQFEK |
| 109 | 220 | [51-LIKK-54 to IIST + N57L] | APTSSSTKKTQLQLEHLLLDLQMILNGINNMNADPELVEFLNRWITFCQSIISTGSLEDLKHLQAL |
| | | | EEELKPLEEVLNLAQSKNFHLRPRDLISNINVIVLELKGSGSGGSGNWSHPQFEK |
| 110 | 221 | [I28M + 51-LIKK-54 to IIST] | APTSSSTKKTQLQLEHLLLDLQMILNGMNNMNADPELVEFLNRWITFCQSIISTGSNEDLKHLQAL |
| | | | EEELKPLEEVLNLAQSKNFHLRPRDLISNINVIVLELKGSGSGGSGNWSHPQFEK |
| 111 | 222 | [I28M + 51-LIKK-54 to IIST + | APTSSSTKKTQLQLEHLLLDLQMILNGMNNMNADPELVEFLNRWITFCQSIISTGSLEDLKHLQAL |
| | | N57L] | EEELKPLEEVLNLAQSKNFHLRPRDLISNINVIVLELKGSGSGGSGNWSHPQFEK |
| 112 | 223 | [51-LIKK-54 to IIST + C65 | APTSSSTKKTQLQLEHLLLDLQMILNGINNMNADPELVEFLNRWITFCQSIISTGSNEDLKHLQNL |
| | | (N-linked glycosylation site)] | SEELKPLEEVLNLAQSKNFHLRPRDLISNINVIVLELKGSGSGGSGNWSHPQFEK |
| 113 | 224 | [I28M + 51-LIKK-54 to IIST + | APTSSSTKKTQLQLEHLLLDLQMILNGMNNMNADPELVEFLNRWITFCQSIISTGSNEDLKHLQNL |
| | | C65(N-linked glycosylation | SEELKPLEEVLNLAQSKNFHLRPRDLISNINVIVLELKGSGSGGSGNWSHPQFEK |
| | | site)] | |
| 114 | 225 | [I28M + 51-LIKK-54 to IIST + | APTSSSTKKTQLQLEHLLLDLQMILNGMNNMNADPELVEFLNRWITFCQSIISTGSLEDLKHLQNL |
| | | N57L + C65(N-linked | SEELKPLEEVLNLAQSKNFHLRPRDLISNINVIVLELKGSGSGGSGNWSHPQFEK |
| | | glycosylation site)] | |
| 115 | 226 | [A48C + C65(N-linked | APTSSSTKKTQLQLEHLLLDLQMILNGINNMNADPELVEFLNRWITFCQSLIKKGSNEDLKHLQNL |
| | | glycosylation site)] | SEELKPLEEVLNLAQSKNFHLRPRDLISNINVIVLELKGSGSGGSGNWSHPQFEK |
| 116 | 227 | [51-LIKKGSNE-58to | APTSSSTKKTQLQLEHLLLDLQMILNGINNMNADPELVEFLNRWITFCQSIISTGSGSDLKHLQAL |
| | | IISTGSGS] | EEELKPLEEVLNLAQSKNFHLRPRDLISNINVIVLELKGSGSGGSGNWSHPQFEK |
| 117 | 228 | [51-LIKKGSNE-58to | APTSSSTKKTQLQLEHLLLDLQMILNGINNMNADPELVEFLNRWITFCQSIISTGSGSDLKHLQNL |
| | | IISTGSGS + C65(N-linked | SEELKPLEEVLNLAQSKNFHLRPRDLISNINVIVLELKGSGSGGSGNWSHPQFEK |
| | | glycosylation site)] | |
| 118 | 229 | [C48S] | APTSSSTKKTQLQLEHLLLDLQMILNGINNMNADPELVEFLNRWITFSQSLIKKGSNEDLKHLQAL |
| | | | EEELKPLEEVLNLAQSKNFHLRPRDLISNINVIVLELKGSGSGGSGNWSHPQFEK |
| 119 | 230 | [C48S + {25, 41 Disulfide}] | APTSSSTKKTQLQLEHLLLDLQMICNGINNMNADPELVEFCNRWITFSQSLIKKGSNEDLKHLQAL |
| | | | EEELKPLEEVLNLAQSKNFHLRPRDLISNINVIVLELKGSGSGGSGNWSHPQFEK |
| 120 | 231 | [{25, 41 Disulfide}] | APTSSSTKKTQLQLEHLLLDLQMICNGINNMNADPELVEFCNRWITFCQSLIKKGSNEDLKHLQAL |
| | | | EEELKPLEEVLNLAQSKNFHLRPRDLISNINVIVLELKGSGSGGSGNWSHPQFEK |
| 121 | 232 | [51-LIKKGSNED-59to | APTSSSTKKTQLQLEHLLLDLQMILNGINNMNADPELVEFLNRWITFCQSIISTLTAGSGSGDLKH |
| | | IISTLTAGSGSG] | LQALEEELKPLEEVLNLAQSKNFHLRPRDLISNINVIVLELKGSGSGGSGNWSHPQFEK |
| 122 | 233 | [51-LIKKGSNED-59to | APTSSSTKKTQLQLEHLLLDLQMILNGINNMNADPELVEFLNRWITFCQSIISTLTAGGSGSGDLK |
| | | IISTLTAGGSGSG] | HLQALEEELKPLEEVLNLAQSKNFHLRPRDLISNINVIVLELKGSGSGGSGNWSHPQFEK |
| 123 | 234 | [51-LIKKGSNED-59to | APTSSSTKKTQLQLEHLLLDLQMILNGINNMNADPELVEFLNRWITFCQSIISTLTAGSGSGDLKH |
| | | IISTLTAGSGSG + C65(N- | LQNLSEELKPLEEVLNLAQSKNFHLRPRDLISNINVIVLELKGSGSGGSGNWSHPQFEK |
| | | linked glycosylation site)] | |
| 124 | 235 | [51-LIKKGSNED-59to | APTSSSTKKTQLQLEHLLLDLQMILNGINNMNADPELVEFLNRWITFCQSIISTLTAGGSGSGDLK |
| | | IISTLTAGGSGSG + C65(N- | HLQNLSEELKPLEEVLNLAQSKNFHLRPRDLISNINVIVLELKGSGSGGSGNWSHPQFEK |
| | | linked glycosylation site)] | |
| 125 | 236 | [51-LIKKGSNEDL-60 to | APTSSSTKKTQLQLEHLLLDLQMILNGINNMNADPELVEFLNRWITFCQSIISTLTAGSGSLEELA |
| | | IISTLTAGSGSLEELA] | KHLQALEEELKPLEEVLNLAQSKNFHLRPRDLISNINVIVLELKGSGSGGSGNWSHPQFEK |
| 126 | 237 | [51-LIKKGSNEDL-60 to | APTSSSTKKTQLQLEHLLLDLQMILNGINNMNADPELVEFLNRWITFCQSIISTLTAGSLEELAKH |
| | | IISTLTAGSLEELA] | LQALEEELKPLEEVLNLAQSKNFHLRPRDLISNINVIVLELKGSGSGGSGNWSHPQFEK |
| 127 | 238 | [51-LIKKGSNEDL-60 to | APTSSSTKKTQLQLEHLLLDLQMILNGINNMNADPELVEFLNRWITFCQSIISTLTAGSGSLEELA |
| | | IISTLTAGSGSLEELA + C65 | KHLQNLSEELKPLEEVLNLAQSKNFHLRPRDLISNINVIVLELKGSGSGGSGNWSHPQFEK |
| | | (N-linkedglycosylation site)] | |
| 128 | 239 | [51-LIKKGSNEDL-60 to | APTSSSTKKTQLQLEHLLLDLQMILNGINNMNADPELVEFLNRWITFCQSIISTLTAGSLEELAKH |
| | | IISTLTAGSLEELA + (N-linked | LQNLSEELKPLEEVLNLAQSKNFHLRPRDLISNINVIVLELKGSGSGGSGNWSHPQFEK |
| | | glycosylation site)] | |
| 129 | 240 | remodelHelix3_1 | APTSSSTKKTQLQLEHLLLDLQMILNGMNNMNADPELVEFLNRIITFCQSLISTGSGSSPEIKEAL |
| | | | QKLEEALKPLEEKLNLAQSKNFHLRPRDLISNINVIVLELKGSGSGGSGNWSHPQFEK |
| 130 | 241 | remodelHelix3_2 | APTSSSTKKTQLQLEHLLLDLQMILNGMNNMNADPELVEFLNRIITFCQSIISTGGVSDPAIKEAL |
| | | | QKLEEALKPLEEKLNLAQSKNFHLRPRDLISNINVIVLELKGSGSGGSGNWSHPQFEK |
| 131 | 242 | remodelHelix3_3 | APTSSSTKKTQLQLEHLLLDLQMILNGMNNMNADPELVEFLNRIITACQSVISTGSDPEELAKLAQ |
| | | | KLEEALKPLEEKLNLAQSKNFHLRPRDLISNINVIVLELKGSGSGGSGNWSHPQFEK |
| 132 | 243 | remodelHelix3_4 | APTSSSTKKTQLQLEHLLLDLQMILNGMNNMNADPELVEFLNRIITLCQSAISTGSVDPEELAKEL |
| | | | QKLEEALKPLEEKLNLAQSKNFHLRPRDLISNINVIVLELKGSGSGGSGNWSHPQFEK |
| 133 | 244 | remodelHelix3_2_A61E | APTSSSTKKTQLQLEHLLLDLQMILNGMNNMNADPELVEFLNRIITFCQSIISTGGVSDPEIKEAL |
| | | | QKLEEALKPLEEKLNLAQSKNFHLRPRDLISNINVIVLELKGSGSGGSGNWSHPQFEK |
| 134 | 245 | remodelHelix3_6 | APTSSSTKKTQLQLEHLLLDLQMILNGMNNMNADPELVEFLNRIITFCQSLISTSGISPEAAKEAQ |
| | | | KLEEALKPLEEKLNLAQSKNFHLRPRDLISNINVIVLELKGSGSGGSGNWSHPQFEK |
| 135 | 246 | coreboundrysel_flexbb_3 | APTSSSIKKIQLLLEHLLLDFQMVLNFLNNANADPELVELVNRALTFCQSIISTGSNEELKHLQAL |
| | | | LENMKPLEEAMNLIQSKNIHIRPRDLISNINVLLLELLGMGSGGSGNWSHPQFEK |
| 136 | 247 | coreboundrysel_flexbb_9 | APTSSSIKKAQLLLEHLLLDFQMILNFLNNVNADPELVELINRILTFCQSIISTGSNEELKHLQAI |
| | | | LETLKPIEEILNLIQSKNIHYRPRDIMSNINVLLLELLGMGSGGSGNWSHPQFEK |
| 137 | 248 | coresel_flexbb_0 | APTSSSTKKTQLQLEHLLLDLQMILNGMNNMNADPELVEFLNRWITFCQSIISTGSNEDLKHLQAL |
| | | | EEELKPLEEVLNLAQSKNFHLRPRDLISNINVLVLELKGSGSGGSGNWSHPQFEK |
| 138 | 249 | coresel_flexbb_12 | APTSSSTKKTQLQLEHLLLDLQMILNGMNNMNADPELVEFLNRWITFCQSIISTGSNEDLKHLQAL |
| | | | EEELKPLEEVLNLAQSKNFHLRPRDLISNINVVVLELKGSGSGGSGNWSHPQFEK |
| 139 | 250 | coresel_flexbb_6 | APTSSSTKKTQLQLEHLLLDLQMILNGMNNMNADPELVEFLNRWITFCQSIISTGSNEDLKHLQAL |
| | | | EEELKPLEEVLNLAQSKNFHLRPRDLISNINVAVLELKGSGSGGSGNWSHPQFEK |
| 140 | 251 | flexbb_11 | APTSSSTKKLQLQLEHLLLDLQMILNGLNNMNADPEAVELMNRIITYCQSIISTGSNEDLKHLQAI |
| | | | EEELKPLEEVMNLAQSKNFHLRPRDLISNINVILLELKGSGSGGSGNWSHPQFEK |
| 141 | 252 | flexbb_12 | APTSSSTKKIQLQLEHLLLDLQMLLNGMNNMNADPEAVELLNRFITFCQSIISTGSNEDLKHLQAL |
| | | | EEELKPIEEVMNLAQSKNFHLRPRDMLSNINVIILELKGSGSGGSGNWSHPQFEK |
| 142 | 253 | flexbb_13 | APTSSSTKKIQLQLEHLLLDLQMVLNGLNNMNADPELVEKLNRIITYCQSIISTGSNEDLKHLQAV |
| | | | EEELKPMEEVLNLAQSKNFHLRPRDMVSNINVILLELKGSGSGGSGNWSHPQFEK |
| 143 | 254 | flexbb_14 | APTSSSTKKLQLQLEHLLLDLQMLLNGMNNMNADPEAVEKLNRMITYCQSIISTGSNEDLKHLQAM |
| | | | EEELKPLEEVLNLAQSKNFHLRPRDMISNMNVILLELKGSGSGGSGNWSHPQFEK |
| 144 | 255 | flexbb_2 | APTSSSTKKLQLQLEHLLLDIQMILNGLNNMNADPEIVEFLNRIITLCQSIISTGSNEDLKHLQAI |
| | | | EEEMKPLEEVMNLAQSKNFHLRPRDLISNINVILLELKGSGSGGSGNWSHPQFEK |
| 145 | 256 | flexbb_3 | APTSSSTKKLQLQLEHLLLDLQMALNGLNNMNADPEMVEMLNRAITFCQSIISTGSNEDLKHLQAI |
| | | | EEEMKPLEEVLNLAQSKNFHLRPRDMVSNINVILLELKGSGSGGSGNWSHPQFEK |
| 146 | 257 | flexbb_6 | APTSSSTKKLQLQLEHLLLDLQMILNGMNNMNADPEIVEILNRAITFCQSIISTGSNEDLKHLQAI |
| | | | EEELKPIEEVLNLAQSKNFHLRPRDMLSNVNVILLELKGSGSGGSGNWSHPQFEK |
| 147 | 258 | Shorter_STII_Linker | APTSSSTKKTQLQLEHLLLDLQMILNGINNMNADPELVEFLNRWITFCQSLIKKGSNEDLKHLQAL |
| | | | EEELKPLEEVLNLAQSKNFHLRPRDLISNINVIVLELKGSGSGNWSHPQFEK |
| 148 | 259 | [51-LIKK-54to IIST + (I28M, | APTSSSTKKTQLQLEHLLLDLQMILNGMNNMNADPELVEFLNRIITFCQSIISTGSLEDLKHLQKL |
| | | N57L, W44I, C65K, E69A, | EEALKPLEEKLNLAQSKNFHLRPRDLISNINVIVLELKGSGSGGSGNWSHPQFEK |
| | | V76K)] | |

TABLE 6
(Variants of IL-2 WT and Variants of Designed Cytokine No. 39)

| | | | Sequence (Designed Cytokines of the disclosure do not comprise or consist of a sequence |
|---|---|---|---|
| Desig- | SEQ | | requiring the bolded and underlined amino acids below, i.e., the bolded and underlined amino |
| nation | ID NO | Features | acids are optional). |
| WT Variant | 260 | WT_IL2_Nterm_STII | NWSHPQFEKGSGGSGAPTSSSTKKTQLQLEHLLLDLQMILNGINNYKNPKLTRMLTFKFYMPKKAT |
| | | | ELKHLQCLEEELKPLEEVLNLAQSKNFHLRPRDLISNINVIVLELKGSETTFMCEYADETATIVEF |
| | | | LNRWITFCQSIISTLT |
| Designed | 261 | Designed Cytokine | NWSHPQFEKGSGSGGSGAPTSSSTKKTQLQLEHLLLDLQMILNGINNMNADPELVEFLNRWITFCQ |
| Cytokine | | No. 39 + | SLIKKGSNEDLKHLQALEEELKPLEEVLNLAQSKNFHLRPRDLISNINVIVLELK |
| No. 150 | | [Nterm_STII] | |
| Designed | 262 | Designed Cytokine | NWSHPQFEKGSGSGGSGAPTSSSTKKTQLQLEHLLLDLQMILNGINNMNADPELVEFLNRWITFAQ |
| Cytokine | | No. 39 + | SLIKKGSNEDLKHLQALEEELKPLEEVLNLAQSKNFHLRPRDLISNINVIVLELK |
| No. 151 | | C48A_Nterm_STII | |

TABLE 7
(Variants of Designed Cytokine No. 132)

| Designed | | | Sequence (Designed Cytokines of the disclosure do not comprise or consist of a sequence |
|---|---|---|---|
| Cytokine | SEQ | | requiringthe bolded and underlined amino acids below, i.e., the bolded and underlined |
| No. | ID NO | Features | amino acids are optional). |
| 152 | 263 | Revert_1_Short_GS | APTSSSTKKTQLQLEHLLLDLQMILNGINNMNADPELVEFLNRWITFCQSAISTGSVDPEELAKEL |
| | | | QKLEEELKPLEEKLNLAQSKNFHLRPRDLISNINVIVLELKGSGSGNWSHPQFEK |
| 153 | 264 | Revert_2_Short_GS | APTSSSTKKTQLQLEHLLLDLQMILNGINNMNADPELVEFLNRWITFCQSIISTGSVDPEELAKEL |
| | | | QKLEEELKPLEEVLNLAQSKNFHLRPRDLISNINVIVLELKGSGSGNWSHPQFEK |
| 154 | 265 | Revert_3_Short_GS | APTSSSTKKTQLQLEHLLLDLQMILNGINNMNADPELVEFLNRWITFCQSIISTGSVDPEELAKEL |
| | | | QKLEEELKPLEEKLNLAQSKNFHLRPRDLISNINVIVLELKGSGSGNWSHPQFEK |
| 155 | 266 | Revert_3_C65pngs_Short_GS | APTSSSTKKTQLQLEHLLLDLQMILNGINNMNADPELVEFLNRWITFCQSIISTGSVDPEELAKEL |
| | | | QNLSEELKPLEEKLNLAQSKNFHLRPRDLISNINVIVLELKGSGSGNWSHPQFEK |
| 156 | 267 | Revert_3_199L_Short_GS | APTSSSTKKTQLQLEHLLLDLQMILNGINNMNADPELVEFLNRWITFCQSIISTGSVDPEELAKEL |
| | | | QKLEEELKPLEEKLNLAQSKNFHLRPRDLISNINVLVLELKGSGSGNWSHPQFEK |
| 157 | 268 | Revert_3_{25, 41DS}_Short_GS | APTSSSTKKTQLQLEHLLLDLQMICNGINNMNADPELVEFCNRWITFCQSIISTGSVDPEELAKEL |
| | | | QKLEEELKPLEEKLNLAQSKNFHLRPRDLISNINVIVLELKGSGSGNWSHPQFEK |
| 158 | 269 | Revert_3_{63, | APTSSSTKKTQLQLEHLLLDLQMILNGINNMNADPELVEFLNRWITFCQSIISTGSVDPEELCKEL |
| | | 108DS}_Short_GS | QKLEEELKPLEEKLNLAQSKNFHLRPRDLISNINVIVLELKCSGSGNWSHPQFEK |
| 159 | 270 | Revert_3_linker_mod_- | APTSSSTKKTQLQLEHLLLDLQMILNGINNMNADPELVEFLNRWITFCQSIISTGSLDPEELAKEL |
| | | 1_Short_GS | QKLEEELKPLEEKLNLAQSKNFHLRPRDLISNINVIVLELKGSGSGNWSHPQFEK |
| 160 | 271 | Revert_3_linker_mod_- | APTSSSTKKTQLQLEHLLLDLQMILNGINNMNADPELVEFLNRWITFCQSIISTGSGSLDPEELAK |
| | | 1_GSPre_Short_GS | ELQKLEEELKPLEEKLNLAQSKNFHLRPRDLISNINVIVLELKGSGSGNWSHPQFEK |
| 161 | 272 | Revert_3_linker_mod_- | APTSSSTKKTQLQLEHLLLDLQMILNGINNMNADPELVEFLNRWITFCQSIISTGSLGSDPEELAK |
| | | 1_GSPost_Short_GS | ELQKLEEELKPLEEKLNLAQSKNFHLRPRDLISNINVIVLELKGSGSGNWSHPQFEK |
| 162 | 273 | Revert_3_linker_mod_1_Short | APTSSSTKKTQLQLEHLLLDLQMILNGINNMNADPELVEFLNRWITFCQSIISTGSLEDPEELAKE |
| | | GS | LQKLEEELKPLEEKLNLAQSKNFHLRPRDLISNINVIVLELKGSGSGNWSHPQFEK |
| 163 | 274 | Revert_3_linker_mod_2_Short | APTSSSTKKTQLQLEHLLLDLQMILNGINNMNADPELVEFLNRWITFCQSIISTLTAGGSVDPEEL |
| | | GS | AKELQKLEEELKPLEEKLNLAQSKNFHLRPRDLISNINVIVLELKGSGSGNWSHPQFEK |
| 164 | 275 | Revert_3_linker_mod_2_VDP > | APTSSSTKKTQLQLEHLLLDLQMILNGINNMNADPELVEFLNRWITFCQSIISTLTAGGSIDPEEL |
| | | IDP_Short_GS | AKELQKLEEELKPLEEKLNLAQSKNFHLRPRDLISNINVIVLELKGSGSGNWSHPQFEK |
| 165 | 276 | Revert_3_linker_mod_2_VDP > | APTSSSTKKTQLQLEHLLLDLQMILNGINNMNADPELVEFLNRWITFCQSIISTLTAGGSLDPEEL |
| | | LDP_Short_GS | AKELQKLEEELKPLEEKLNLAQSKNFHLRPRDLISNINVIVLELKGSGSGNWSHPQFEK |
| 166 | 277 | Revert_3_linker_mod_2_G27A_ | APTSSSTKKTQLQLEHLLLDLQMILNAINNMNADPELVEFLNRWITFCQSIISTLTAGGSVDPEEL |
| | | Short_GS | AKELQKLEEELKPLEEKLNLAQSKNFHLRPRDLISNINVIVLELKGSGSGNWSHPQFEK |
| 167 | 278 | Revert_3_linker_mod_2_L79E_ | APTSSSTKKTQLQLEHLLLDLQMILNGINNMNADPELVEFLNRWITFCQSIISTLTAGGSVDPEEL |
| | | Short_GS | AKELQKLEEELKPLEEKLNEAQSKNFHLRPRDLISNINVIVLELKGSGSGNWSHPQFEK |
| 168 | 279 | Revert_3_linker_mod_3_Short_ | APTSSSTKKTQLQLEHLLLDLQMILNGINNMNADPELVEFLNRWITFCQSIISTLTAGSGSVDPEE |
| | | GS | LAKELQKLEEELKPLEEKLNLAQSKNFHLRPRDLISNINVIVLELKGSGSGNWSHPQFEK |

TABLE 8
(Variants of Designed Cytokine No. 109)

| Designed | | | Sequence (Designed Cytokines of the disclosure do not comprise or consist of a sequence |
|---|---|---|---|
| Cytokine | SEQ | | requiring the bolded and underlined amino acids below, i.e., the bolded and underlined amino |
| No. | ID NO | Features | acids are optional). |
| 169 | 280 | Short_GS | APTSSSTKKTQLQLEHLLLDLQMILNGINNMNADPELVEFLNRWITFCQSIISTGSLEDLKHLQAL |
| | | | EEELKPLEEVLNLAQSKNFHLRPRDLISNINVIVLELKGSGSGNWSHPQFEK |
| 170 | 281 | Shorter_GSG | APTSSSTKKTQLQLEHLLLDLQMILNGINNMNADPELVEFLNRWITFCQSIISTGSLEDLKHLQAL |
| | | | EEELKPLEEVLNLAQSKNFHLRPRDLISNINVIVLELKGSGNWSHPQFEK |
| 171 | 282 | Shortest_GS | APTSSSTKKTQLQLEHLLLDLQMILNGINNMNADPELVEFLNRWITFCQSIISTGSLEDLKHLQAL |
| | | | EEELKPLEEVLNLAQSKNFHLRPRDLISNINVIVLELKGSNWSHPQFEK |
| 172 | 283 | DP_init_Short_GS | APTSSSTKKTQLQLEHLLLDLQMILNGINNMNADPELVEFLNRWITFCQSIISTGSLDPEDLKHLQ |
| | | | ALEEELKPLEEVLNLAQSKNFHLRPRDLISNINVIVLELKGSGSGNWSHPQFEK |
| 173 | 284 | {25, 41DS}_Short_GS | APTSSSTKKTQLQLEHLLLDLQMICNGINNMNADPELVEFCNRWITFCQSIISTGSLEDLKHLQAL |
| | | | EEELKPLEEVLNLAQSKNFHLRPRDLISNINVIVLELKGSGSGNWSHPQFEK |

TABLE 9
(Variants of Designed Cytokine No. 124)

| Designed | | | Sequence (Designed Cytokines of the disclosure do not comprise or consist of a sequence |
|---|---|---|---|
| Cytokine | SEQ | | requiring the bolded and underlined amino acids below, i.e., the bolded and underlined amino |
| No. | ID NO | Features | acids are optional). |
| 174 | 285 | Short_GS | APTSSSTKKTQLQLEHLLLDLQMILNGINNMNADPELVEFLNRWITFCQSIISTLTAGGSGSGDLK |
| | | | HLQNLSEELKPLEEVLNLAQSKNFHLRPRDLISNINVIVLELKGSGSGNWSHPQFEK |
| 175 | 286 | GGSGSG > GGSLSG_Short_GS | APTSSSTKKTQLQLEHLLLDLQMILNGINNMNADPELVEFLNRWITFCQSIISTLTAGGSLSGDLK |
| | | | HLQNLSEELKPLEEVLNLAQSKNFHLRPRDLISNINVIVLELKGSGSGNWSHPQFEK |
| 176 | 287 | GGSGSG > GGSLSPE_Short_GS | APTSSSTKKTQLQLEHLLLDLQMILNGINNMNADPELVEFLNRWITFCQSIISTLTAGGSLSPEDL |
| | | | KHLQNLSEELKPLEEVLNLAQSKNFHLRPRDLISNINVIVLELKGSGSGNWSHPQFEK |
| 177 | 288 | GGSGSG > GGSLGSG_Short_GS | APTSSSTKKTQLQLEHLLLDLQMILNGINNMNADPELVEFLNRWITFCQSIISTLTAGGSLGSGDL |
| | | | KHLQNLSEELKPLEEVLNLAQSKNFHLRPRDLISNINVIVLELKGSGSGNWSHPQFEK |
| 178 | 289 | LKHL > LAKHL_Short_GS | APTSSSTKKTQLQLEHLLLDLQMILNGINNMNADPELVEFLNRWITFCQSIISTLTAGGSGSGDLA |
| | | | KHLQNLSEELKPLEEVLNLAQSKNFHLRPRDLISNINVIVLELKGSGSGNWSHPQFEK |

TABLE 10
(Variants of N-terminus of WT IL-2 and of Designed Cytokines)

| Designed | | | Sequence (Designed Cytokines of the disclosure do not comprise or consist of a sequence |
|---|---|---|---|
| Cytokine | SEQ | | requiring the bolded and underlined amino acids below, i.e., the bolded and underlined amino |
| No. | ID NO | Features | acids are optional). |
| 179 | 290 | Designed Cytokine No. | STKKTQLQLEHLLLDLQMILNGINNMNADPELVEFLNRWITFCQSLIKKGSNEDLKHLQALEEELK |
| | | 39_Truncated_Nterm | PLEEVLNLAQSKNFHLRPRDLISNINVIVLELKGSGSGGSGNWSHPQFEK |
| Variant of | 291 | IL-2 WT_Truncated_Nterm | STKKTQLQLEHLLLDLQMILNGINNYKNPKLTRMLTFKFYMPKKATELKHLQCLEEELKPLEEVLN |
| IL-2 | | | LAQSKNFHLRPRDLISNINVIVLELKGSETTFMCEYADETATIVEFLNRWITFCQSIISTLTGSGG |
| | | | SGNWSHPQFEK |
| 181 | 292 | Designed Cytokine No. | STKKTQLQLEHLLLDLQMILNGINNMNADPELVEFLNRWITFCQSIISTGSLEDLKHLQALEEELK |
| | | 109_Truncated_Nterm | PLEEVLNLAQSKNFHLRPRDLISNINVIVLELKGSGSGGSGNWSHPQFEK |
| 182 | 293 | Designed Cytokine No. | STKKTQLQLEHLLLDLQMILNGINNMNADPELVEFLNRWITFCQSIISTGSVDPEELAKELQKLEE |
| | | 154_Truncated_Nterm | ELKPLEEKLNLAQSKNFHLRPRDLISNINVIVLELKGSGSGNWSHPQFEK |
| 183 | 294 | Designed Cytokine No. | DPKKTQLQLEHLLLDLQMILNGINNMNADPELVEFLNRWITFCQSIISTGSVDPEELAKELQKLEE |
| | | 154_DP_Nterm | ELKPLEEKLNLAQSKNFHLRPRDLISNINVIVLELKGSGSGNWSHPQFEK |

TABLE 11
(Variants of WT IL-2)

| | | | Sequence (Designed Cytokines of the disclosure do not comprise or consist of a |
|---|---|---|---|
| IL-2 | SEQ | | sequence requiring the bolded and underlined amino acids below, |
| Variant No. | ID NO | Features | i.e., the bolded and underlined amino acids are optional). |
| 1 | 295 | WT_IL2_L72R_STII | APTSSSTKKTQLQLEHLLLDLQMILNGINNYKNPKLTRMLTFKFYMPKKATELKHLQCLEEELKPLEEV |
| | | | LNRAQSKNFHLRPRDLISNINVIVLELKGSETTFMCEYADETATIVEFLNRWITFCQSIISTLTGSGGS |
| | | | GNWSHPQFEK |
| 2 | 296 | WT_IL2_L72Q_STII | APTSSSTKKTQLQLEHLLLDLQMILNGINNYKNPKLTRMLTFKFYMPKKATELKHLQCLEEELKPLEEV |
| | | | LNQAQSKNFHLRPRDLISNINVIVLELKGSETTFMCEYADETATIVEFLNRWITFCQSIISTLTGSGGS |
| | | | GNWSHPQFEK |
| 3 | 297 | WT_IL2_L72E_STII | APTSSSTKKTQLQLEHLLLDLQMILNGINNYKNPKLTRMLTFKFYMPKKATELKHLQCLEEELKPLEEV |
| | | | LNEAQSKNFHLRPRDLISNINVIVLELKGSETTFMCEYADETATIVEFLNRWITFCQSIISTLTGSGGS |
| | | | GNWSHPQFEK |
| 4 | 298 | WT_IL2_L72N_L74S_STII | APTSSSTKKTQLQLEHLLLDLQMILNGINNYKNPKLTRMLTFKFYMPKKATELKHLQCLEEELKPLEEV |
| | | | LNNASSKNFHLRPRDLISNINVIVLELKGSETTFMCEYADETATIVEFLNRWITFCQSIISTLTGSGGS |
| | | | GNWSHPQFEK |
| 5 | | WT IL-2 cterm STII tag | APTSSSTKKTQLQLEHLLLDLQMILNGINNYKNPKLTRMLTFKFYMPKKATELKHLQCLEEELKPLEEV |
| | | | LNLAQSKNFHLRPRDLISNINVIVLELKGSETTFMCEYADETATIVEFLNRWITFCQSIISTLTGSGGS |
| | | | GNWSHPQFEK |

TABLE 12
(Variants of Designed Cytokine No. 154)

| Designed | | | Sequence (Designed Cytokines of the disclosure do not comprise or consist of a sequence |
|---|---|---|---|
| Cytokine | SEQ | | requiring the bolded and underlined amino acids below, i.e., the bolded and underlined |
| No. | ID NO | Features | amino acids are optional). |
| 188 | 299 | Designed Cytokine No. | DPKKTQLQLEHLLLDLQMILNGINNMNADPELVEFLNRWITFCQSIISTGSVDPEELAKELQKLEEELK |
| | | 154_DP_Nterm | PLEEKLNLAQSKNFHLRPRDLISNINVIVLELKGSGSGNWSHPQFEK |

TABLE 13
(Variants of Designed Cytokine No. 153)

| Designed | | | Sequence (Designed Cytokines of the disclosure do not comprise or consist of a |
|---|---|---|---|
| Cytokine | SEQ | | sequence requiring the bolded and underlined amino acids below, i.e., the bolded |
| No. | ID NO | Features | and underlined amino acids are optional). |
| 189 | 300 | Designed Cytokine No. 153 I99L | APTSSSTKKTQLQLEHLLLDLQMILNGINNMNADPELVEFLNRWITFCQSIISTGSVDPEELAK |
| | | | ELQKLEEELKPLEEVLNLAQSKNFHLRPRDLISNINVLVLELKGSGSGNWSHPQFEK |
| 190 | 30 | Designed Cytokine No. 153 | APTSSSTKKTQLQLEHLLLDLQMILNGINNMNADPELVEFLNRWITFCQSIISTGSVDPEELAK |
| | | rE65H + I99L | HLQKLEEELKPLEEVLNLAQSKNFHLRPRDLISNINVLVLELKGSGSGNWSHPQFEK |
| 191 | 302 | Designed Cytokine No. 153_ | APTSSSTKKTQLQLEHLLLDLQMILNGINNMNADPELVEFLNRWITFCQSIISTGSVDPEELKE |
| | | d63A + I99L | LQKLEEELKPLEEVLNLAQSKNFHLRPRDLISNINVLVLELKGSGSGNWSHPQFEK |
| 192 | 303 | Designed Cytokine No. 153_ | APTSSSTKKTQLQLEHLLLDLQMILNGINNMNADPELVEFLNRWITFCQSIISTGSVDPEELKH |
| | | d63A_rE65H + I99L | LQKLEEELKPLEEVLNLAQSKNFHLRPRDLISNINVLVLELKGSGSGNWSHPQFEK |
| 193 | 304 | Designed Cytokine No. 153 | APTSSSTKKTQLQLEHLLLDLQMILNGINNMNADPELVEFLNRWITFCQSIISTGSVDPEELKH |
| | | d63A_rE65H_rK67A + I99L | LQALEEELKPLEEVLNLAQSKNFHLRPRDLISNINVLVLELKGSGSGNWSHPQFEK |
| 209 | 320 | QSTII-GSGSG-(Designed | QNWSHPQFEKGSGSGAPTSSSTKKTQLQLEHLLLDLQMILNGINNMNADPELVEFLNRWITFCQ |
| | | Cytokine No. 153) + I99L | SIISTGSVDPEELAKELQKLEEELKPLEEVLNLAQSKNFHLRPRDLISNINVLVLELK |
| 210 | 321 | QSTII-GSGSG-Nterm-trunc-- | QNWSHPQFEKGSGSGDPTKKTQLQLEHLLLDLQMILNGINNMNADPELVEFLNRWITFCQSIIS |
| | | (Designed Cytokine No. 153) + I99L | TGSVDPEELAKELQKLEEELKPLEEVLNLAQSKNFHLRPRDLISNINVLVLELK |
| 192 | 322 | Designed Cytokine No. 153 | APTSSSTKKTQLQLEHLLLDLQMILNGINNMNADPELVEFLNRWITFCQSIISTGSVDPEELKH |
| | | d63A_rE65H_rK67A | LQALEEELKPLEEVLNLAQSKNFHLRPRDLISNINVIVLELKGSGSGNWSHPQFEK |
| 193 | 323 | Designed Cytokine No. 153 + | APTSSSTKKTQLQLEHLLLDLQMILNGINNMNADPELVEFLNRWITFCQSIISTGSVDPEELAK |
| | | R91S + I99L | ELQKLEEELKPLEEVLNLAQSKNFHLSPRDLISNINVLVLELKGSGSGNWSHPQFEK |
| 194 | 324 | Designed Cytokine No. 153 + R91S | APTSSSTKKTQLQLEHLLLDLQMILNGINNMNADPELVEFLNRWITFCQSIISTGSVDPEELAK |
| | | | ELQKLEEELKPLEEVLNLAQSKNFHLSPRDLISNINVIVLELKGSGSGNWSHPQFEK |
| 195 | 325 | Designed Cytokine No. 153 + | APTSSSTKKTQLQLEHLLLDLQMILNGINNMNADPELVEFLNRWITFCQSIISTGSVDPEELAK |
| | | R91N + I99L | ELQKLEEELKPLEEVLNLAQSKNFHLNPRDLISNINVLVLELKGSGSGNWSHPQFEK |
| 196 | 326 | Designed Cytokine No. 153_ | APTSSSTKKTQLQLEHLLLDLQMILNGINNMNADPELVEFLNRWITFCQSIISTGSVDPEELKE |
| | | d63A + R91S + I99L | LQKLEEELKPLEEVLNLAQSKNFHLSPRDLISNINVLVLELKGSGSGNWSHPQFEK |
| 197 | 327 | Designed Cytokine No. 153_ | APTSSSTKKTQLQLEHLLLDLQMILNGINNMNADPELVEFLNRWITFCQSIISTGSVDPEELKE |
| | | d63A + R91N + I99L | LQKLEEELKPLEEVLNLAQSKNFHLNPRDLISNINVLVLELKGSGSGNWSHPQFEK |
| 198 | 328 | Designed Cytokine No. 153 | APTSSSTKKTQLQLEHLLLDLQMILNGINNMNADPELVEFLNRWITFCQSIISTGSVDPEELKH |
| | | d63A_rE65H_rK67A + R91S + I99L | LQALEEELKPLEEVLNLAQSKNFHLSPRDLISNINVLVLELKGSGSGNWSHPQFEK |
| 199 | 329 | Designed Cytokine No. 153 | APTSSSTKKTQLQLEHLLLDLQMILNGINNMNADPELVEFLNRWITFCQSIISTGSVDPEELKH |
| | | d63A_rE65H_rK67A + R91N + I99L | LQALEEELKPLEEVLNLAQSKNFHLNPRDLISNINVLVLELKGSGSGNWSHPQFEK |
| 200 | 330 | Designed Cytokine No. 153 | APTSSSTKKTQLQLEHLLLDLQMILNGINNMNADPELVEFLNRWITFCQSIISTGSVDPEELKH |
| | | d63A_rE65H_rK67A + R91S | LQALEEELKPLEEVLNLAQSKNFHLSPRDLISNINVIVLELKGSGSGNWSHPQFEK |
| 220 | 331 | Designed Cytokine No. 153 L1- | APTSSSTKKTQLQLEHLLLDLQMILNGINNMNLEPELIDELNRWITFCQSIISTGSVDPELAAK |
| | | 1_L2-1_deimmunized | ELDKLEEELKPLEEVLNLAQSKNFHLRPRDLISNINVIVLELKGSGSGNWSHPQFEK |
| 221 | 332 | Designed Cytokine No. 153 L1- | APTSSSTKKTQLQLEHLLLDLQMILNGINNMNLEPELIDELNRWITFCQSIISTGSVDPEVSAS |
| | | 1_L2-2_deimmunized | ELDKLEEELKPLEEVLNLAQSKNFHLRPRDLISNINVIVLELKGSGSGNWSHPQFEK |
| 222 | 333 | Designed Cytokine No. 153 L1- | APTSSSTKKTQLQLEHLLLDLQMILNGINNQNGDPELVELLNRWITFCQSIISTGSVDPELAAK |
| | | 2_L2-1_deimmunized | ELDKLEEELKPLEEVLNLAQSKNFHLRPRDLISNINVIVLELKGSGSGNWSHPQFEK |
| 223 | 334 | Designed Cytokine No. 153 L1- | APTSSSTKKTQLQLEHLLLDLQMILNGINNQNGDPELVELLNRWITFCQSIISTGSVDPEVSAS |
| | | 2_L2-2_deimmunized | ELDKLEEELKPLEEVLNLAQSKNFHLRPRDLISNINVIVLELKGSGSGNWSHPQFEK |
| 224 | 335 | Designed Cytokine No. 153 L1- | APTSSSTKKTQLQLEHLLLDLQMILNGINEMNADPELVDLLNRWITFCQSIISTGSVDPELAAK |
| | | 3_L2-1_deimmunized | ELDKLEEELKPLEEVLNLAQSKNFHLRPRDLISNINVIVLELKGSGSGNWSHPQFEK |
| 225 | 336 | Designed Cytokine No. 153 L1- | APTSSSTKKTQLQLEHLLLDLQMILNGINEMNADPELVDLLNRWITFCQSIISTGSVDPEVSAS |
| | | 3_L2-2_deimmunized | ELDKLEEELKPLEEVLNLAQSKNFHLRPRDLISNINVIVLELKGSGSGNWSHPQFEK |
| 226 | 337 | Designed Cytokine No. 153 L1- | APTSSSTKKTQLQLEHLLLDLQMILNGINNINGDPELVNFLNRWITFCQSIISTGSVDPELAAK |
| | | 4_L2-1_deimmunized | ELDKLEEELKPLEEVLNLAQSKNFHLRPRDLISNINVIVLELKGSGSGNWSHPQFEK |
| 227 | 338 | Designed Cytokine No. 153 L1- | APTSSSTKKTQLQLEHLLLDLQMILNGINNINGDPELVNFLNRWITFCQSIISTGSVDPEVSAS |
| | | 4_L2-2_deimmunized | ELDKLEEELKPLEEVLNLAQSKNFHLRPRDLISNINVIVLELKGSGSGNWSHPQFEK |
| 228 | 339 | Designed Cytokine No. 153 L1- | APTSSSTKKTQLQLEHLLLDLQMILNGINNMNGDPELTEMLNRWITFCQSIISTGSVDPELAAK |
| | | 6_L2-1_deimmunized | ELDKLEEELKPLEEVLNLAQSKNFHLRPRDLISNINVIVLELKGSGSGNWSHPQFEK |
| 229 | 340 | Designed Cytokine No. 153 L1- | APTSSSTKKTQLQLEHLLLDLQMILNGINNMNGDPELTEMLNRWITFCQSIISTGSVDPEVSAS |
| | | 6_L2-2_deimmunized | ELDKLEEELKPLEEVLNLAQSKNFHLRPRDLISNINVIVLELKGSGSGNWSHPQFEK |

TABLE 14
(Variants of Designed Cytokine No. 169)

| Designed | | | Sequence (Designed Cytokines of the disclosure do not comprise or consist of a |
|---|---|---|---|
| Cytokine | SEQ | | sequence requiring the bolded and underlined amino acids below, i.e., the bolded |
| No. | ID NO | Features | and underlined amino acids are optional). |
| 194 | 305 | Designed Cytokine No. 169 + I99L | APTSSSTKKTQLQLEHLLLDLQMILNGINNMNADPELVEFLNRWITFCQSIISTGSLEDLKHLQ |
| | | | ALEEELKPLEEVLNLAQSKNFHLRPRDLISNINVLVLELKGSGSGNWSHPQFEK |
| 195 | 306 | Designed Cytokine No. 169 + | APTSSSTKKTQLQLEHLLLDLQMILNGINNMNADPELVEFLNRWITFCQSIISTGSLEDLKHLQ |
| | | I99L + FactorXaShort | ALEEELKPLEEVLNLAQSKNFHLRPRDLISNINVLVLELKGSIEGRGSGNWSHPQFEK |
| 196 | 307 | Designed Cytokine No. 169 + I99F | APTSSSTKKTQLQLEHLLLDLQMILNGINNMNADPELVEFLNRWITFCQSIISTGSLEDLKHLQ |
| | | | ALEEELKPLEEVLNLAQSKNFHLRPRDLISNINVFVLELKGSGSGNWSHPQFEK |
| 197 | 308 | Designed Cytokine No. 169 + I99Y | APTSSSTKKTQLQLEHLLLDLQMILNGINNMNADPELVEFLNRWITFCQSIISTGSLEDLKHLQ |
| | | | ALEEELKPLEEVLNLAQSKNFHLRPRDLISNINVYVLELKGSGSGNWSHPQFEK |
| 198 | 309 | Designed Cytokine No. 169 + | APTSSSTKKTQLQLEHLLLDLQMILNGINNMNADPELVEFLNRWITFCQSIISTGSLEDLKHLQ |
| | | I99L + L3-4 Truncation | ALEEELKPLEEVLNLAQKNFHLRPRDLISNINVLVLELKGSGSGNWSHPQFEK |
| 199 | 310 | Designed Cytokine No. 169 + | APTSSSTKKTQLQLEHLLLDLQMILNGINNMNADPELVEFLNRWITFCQSIISTGSLEDLKHLQ |
| | | I99L + R81D | ALEEELKPLEEVLNLAQSKNFHLDPRDLISNINVLVLELKGSGSGNWSHPQFEK |
| 200 | 311 | Designed Cytokine No. 169 + | APTSSSTKKTQLQLEHLLLDLQMILNGINNMNADPELVEFLNRWITFCQSIISTGSLEDLKHLQ |
| | | I99L + R81S | ALEEELKPLEEVLNLAQSKNFHLSPRDLISNINVLVLELKGSGSGNWSHPQFEK |
| 201 | 312 | Designed Cytokine No. 169 + | APTSSSTKKTQLQLEHLLLDLQMILNGINNMNADPELVEFLNRWITFCQSIISTGSLEDLKHLQ |
| | | I99L + R81N | ALEEELKPLEEVLNLAQSKNFHLNPRDLISNINVLVLELKGSGSGNWSHPQFEK |
| 202 | 313 | Designed Cytokine No. 169 + | APTSSSTKKTQLQLEHLLLDLQMILNGINNMNADPELVEFLNRWITFCQSIISTGSLEDLKHLQ |
| | | I99L + R81N + L85I | ALEEELKPLEEVLNLAQSKNFHLNPRDIISNINVLVLELKGSGSGNWSHPQFEK |
| 203 | 314 | Designed Cytokine No. 169 + | APTSSSTKKTQLQLEHLLLDLQMILNGINNMNADPELVEFLNRWITFCQSIISTGSLEDLKHLQ |
| | | I99L + R81N + L85A | ALEEELKPLEEVLNLAQSKNFHLNPRDAISNINVLVLELKGSGSGNWSHPQFEK |
| 204 | 315 | Designed Cytokine No. 159 + | APTSSSTKKTQLQLEHLLLDLQMILNGINNMNADPELVEFLNRWITFCQSIISTGSLEDLKHLQ |
| | | IL2Rb Mutations | ALEEELKPLEEVLNLAQSKNFHFDPRDVVSNINVFVLELKGSGSGNWSHPQFEK |
| 205 | 316 | Designed Cytokine No. 169 + | APTSSSTKKTQLQLEHLLLDLQMILNGINNMNADPELVEFLNRWITFCQSIISTGSIEDLKHLQ |
| | | L57I + I99L | ALEEELKPLEEVLNLAQSKNFHLRPRDLISNINVLVLELKGSGSGNWSHPQFEK |
| 206 | 317 | Designed Cytokine No. 169 + | APTSSSTKKTQLQLEHLLLDLQMILNGINNMNADPELVEFLNRWITFCQSIISTGSVEDLKHLQ |
| | | L57V + I99L | ALEEELKPLEEVLNLAQSKNFHLRPRDLISNINVLVLELKGSGSGNWSHPQFEK |
| 207 | 318 | QSTII-GSGSG-(Designed | QNWSHPQFEKGSGSGAPTSSSTKKTQLQLEHLLLDLQMILNGINNMNADPELVEFLNRWITFCQ |
| | | Cytokine No. 159) + I99L | SIISTGSLEDLKHLQALEEELKPLEEVLNLAQSKNFHLRPRDLISNINVLVLELK |
| 208 | 319 | QSTII-GSGSG-Nterm-trunc- | QNWSHPQFEKGSGSGDPTKKTQLQLEHLLLDLQMILNGINNMNADPELVEFLNRWITFCQSIIS |
| | | (Designed Cytokine No. 169) + | TGSLEDLKHLQALEEELKPLEEVLNLAQSKNFHLRPRDLISNINVLVLELK |
| | | I99L | |
| 230 | 341 | Designed Cytokine No. 169_L1- | APTSSSTKKTQLQLEHLLLDLQMILNGINNMNLEPELIDELNRWITFCQSIISTGSLEQLPELQ |
| | | 1_L2-1_deimmunized | LLEEELKPLEEVLNLAQSKNFHLRPRDLISNINVIVLELKGSGSGNWSHPQFEK |
| 231 | 342 | Designed Cytokine No. 169_L1- | APTSSSTKKTQLQLEHLLLDLQMILNGINNQNGDPELVELLNRWITFCQSIISTGSLEQLPELQ |
| | | 2_L2-1_deimmunized | LLEEELKPLEEVLNLAQSKNFHLRPRDLISNINVIVLELKGSGSGNWSHPQFEK |
| 232 | 343 | Designed Cytokine No. 169_L1- | APTSSSTKKTQLQLEHLLLDLQMILNGINEMNADPELVDLLNRWITFCQSIISTGSLEQLPELQ |
| | | 3_L2-1_deimmunized | LLEEELKPLEEVLNLAQSKNFHLRPRDLISNINVIVLELKGSGSGNWSHPQFEK |
| 233 | 344 | Designed Cytokine No. 169_L1- | APTSSSTKKTQLQLEHLLLDLQMILNGINNINGDPELVNFLNRWITFCQSIISTGSLEQLPELQ |
| | | 4_L2-1_deimmunized | LLEEELKPLEEVLNLAQSKNFHLRPRDLISNINVIVLELKGSGSGNWSHPQFEK |
| 234 | 345 | Designed Cytokine No. 169_L1- | APTSSSTKKTQLQLEHLLLDLQMILNGINNMNGDPELTEMLNRWITFCQSIISTGSLEQLPELQ |
| | | 6_L2-1_deimmunized | LLEEELKPLEEVLNLAQSKNFHLRPRDLISNINVIVLELKGSGSGNWSHPQFEK |

TABLE 15
(Variants of Designed Cytokine No. 182)

| Designed | | | Sequence (Designed Cytokines of the disclosure do not comprise or consist of a |
|---|---|---|---|
| Cytokine | SEQ | | sequence requiring the bolded and underlined amino acids below, i.e., the bolded |
| No. | ID NO | Features | and underlined amino acids are optional). |
| 235 | 346 | Designed Cytokine No. 182_L1- | APTSSSTKKTQLQLEHLLLDLQMILNGINNMNLEPELIDELNRWITFCQSIISTGSLEQLPELQ |
| | | 1_L2-1_deimmunized | LLEEELKPLEEVLNLAQSKNFHLNPRDLISNINVLVLELKGSGSGNWSHPQFEK |
| 236 | 347 | Designed Cytokine No. 182_L1- | APTSSSTKKTQLQLEHLLLDLQMILNGINNQNGDPELVELLNRWITFCQSIISTGSLEQLPELQ |
| | | 2_L2-1_deimmunized | LLEEELKPLEEVLNLAQSKNFHLNPRDLISNINVLVLELKGSGSGNWSHPQFEK |
| 237 | 348 | Designed Cytokine No. 182_L1- | APTSSSTKKTQLQLEHLLLDLQMILNGINEMNADPELVDLLNRWITFCQSIISTGSLEQLPELQ |
| | | 3_L2-1_deimmunized | LLEEELKPLEEVLNLAQSKNFHLNPRDLISNINVLVLELKGSGSGNWSHPQFEK |
| 238 | 349 | Designed Cytokine No. 182_L1- | APTSSSTKKTQLQLEHLLLDLQMILNGINNINGDPELVNFLNRWITFCQSIISTGSLEQLPELQ |
| | | 4_L2-1_deimmunized | LLEEELKPLEEVLNLAQSKNFHLNPRDLISNINVLVLELKGSGSGNWSHPQFEK |
| 239 | 350 | Designed Cytokine No. 182 L1- | APTSSSTKKTQLQLEHLLLDLQMILNGINNMNGDPELTEMLNRWITFCQSIISTGSLEQLPELQ |
| | | 6_L2-1_deimmunized | LLEEELKPLEEVLNLAQSKNFHLNPRDLISNINVLVLELKGSGSGNWSHPQFEK |

### Nucleic Acids

In some embodiments of the disclosure, the terms “Nucleic acid,” “nucleic acid molecule,” “nucleotide,” “nucleotide sequence,” “polynucleotide,” and grammatical variants thereof are used interchangeably and refer to the phosphate ester polymeric form of ribonucleosides (adenosine, guanosine, uridine or cytidine; “RNA molecules”) or deoxyribonucleosides (deoxyadenosine, deoxyguanosine, deoxythymidine, or deoxycytidine; “DNA molecules”), or any phosphoester analogs thereof, such as phosphorothioates and thioesters, in either single stranded form, or a double-stranded helix. Single stranded nucleic acid sequences refer to single-stranded DNA (ssDNA) or single-stranded RNA (ssRNA). Double stranded DNA-DNA, DNA-RNA and RNA-RNA helices are possible.

In some embodiments of the disclosure, “Nucleic acid,” and in particular a DNA or RNA molecule, refers only to the primary and secondary structure of the molecule, and does not limit it to any particular tertiary forms. In some embodiments of the disclosure, “Nucleic acid,” includes double-stranded DNA found, inter alia, in linear or circular DNA molecules (e.g., restriction fragments), plasmids, supercoiled DNA and chromosomes. In discussing the structure of particular double-stranded DNA molecules, sequences are provided according to the normal convention of writing the sequence left to right in the 5′ to 3′ direction along the non-transcribed strand of DNA (i.e., the strand having a sequence homologous to the messenger RNA or mRNA). Unless otherwise indicated, all nucleic acid and nucleotide sequences are written left to right in 5′ to 3′ orientation.

Nucleotides are referred to by their commonly known one-letter symbols recommended by the IUPAC-IUB Biochemical Nomenclature Commission. Accordingly, ‘A’ represents adenine, ‘C’ represents cytosine, ‘G’ represents guanine, ‘T’ represents thymine, and ‘U’ represents uracil.

In some embodiments of the disclosure, the term “Polynucleotide” refers to polymers of nucleotides of any length or type, including ribonucleotides, deoxyribonucleotides, analogs thereof, or mixtures thereof. This term refers to the primary structure of the molecule. Thus, the term includes triple-, double- and single-stranded deoxyribonucleic acid (“DNA”) and ribonucleic acid (“RNA”). It also includes modified, for example by alkylation and/or by capping, and unmodified forms of the polynucleotide. More particularly, “polynucleotide” includes polydeoxyribonucleotides (containing 2-deoxy-D-ribose) and polyribonucleotides (containing D-ribose), including mRNA, whether spliced or unspliced, any other type of polynucleotide which is an N- or C-glycoside of a purine or pyrimidine base, and other polymers containing nucleotide backbones, for example, polyamide (e.g., peptide nucleic acids “PNAs”) and polymorpholino polymers, and other synthetic sequence-specific nucleic acid polymers providing that the polymers contain nucleobases in a configuration which allows for base pairing and base stacking, such as is found in DNA and RNA.

In some embodiments of the disclosure, a polynucleotide comprises a DNA sequence. In some embodiments of the disclosure, a polynucleotide comprises a DNA sequence inserted in a vector or a vector comprising a DNA sequence.

In some embodiments of the disclosure, a polynucleotide comprises an mRNA. In some embodiments, the mRNA is a synthetic mRNA or the mRNA comprises a synthetic nucleotide.

In some embodiments of the disclosure, a polynucleotide comprises at least one unnatural, non-naturally occurring or modified nucleic acid. In some embodiments, the polynucleotide comprises a plurality of unnatural, non-naturally occurring or modified nucleic acids. In some embodiments, all nucleic acids of a certain class are unnatural, non-naturally occurring or modified nucleic acids (e.g., all uridines in a polynucleotide can be replaced with an unnatural nucleobase, e.g., 5-methoxy uridine).

In some embodiments of the disclosure, “expression” refers to the transcription and/or translation of a particular nucleotide sequence driven by a promoter.

In some embodiments of the disclosure, “expression vector” refers to a plasmid, virus, or other nucleic acid designed for polypeptide expression in a cell. The vector or construct is used to introduce a gene into a host cell whereby the vector will interact with polymerases in the cell to express the protein encoded in the vector/construct. The expression vector may exist in the cell extrachromosomally or may be integrated into the chromosome. Expression vectors may include additional sequences which render the vector suitable for replication and integration in prokaryotes, eukaryotes, or preferably both (e.g., shuttle vectors). The polynucleotides of the disclosure may be provided as components of expression vectors.

In some embodiments of the disclosure, “cloning vector” refers to a plasmid, virus, or other nucleic acid designed for producing copies of a polynucleotide. Cloning vectors may contain transcription and translation initiation sequences, transcription and translation termination sequences and a polyadenylation signal. Such constructs will typically include a 5′ LTR, a tRNA binding site, a packaging signal, an origin of second-strand DNA synthesis, and a 3′ LTR or a portion thereof. The polynucleotides of the disclosure may be provided as components of cloning vectors, which may be used to produce the polynucleotides of the disclosure.

In some embodiments of the disclosure, “encoding” or the like refers to the capacity of specific sequences of nucleotides in a polynucleotide (e.g. a gene, cDNA, or mRNA) to serve as templates for synthesis of other polymers and macromolecules in biological processes having either a defined sequence of nucleotides (e.g., rRNA, tRNA and mRNA) or a defined sequence of amino acids. Thus, a gene, cDNA, or RNA, encodes a protein if transcription and translation of mRNA corresponding to that gene produces the protein in a cell or other biological system. Both the coding strand, the nucleotide sequence of which is identical to the mRNA sequence and is usually provided in sequence listings, and the non-coding strand, used as the template for transcription of a gene or cDNA, can be referred to as encoding the protein or other product of that gene or cDNA.

Unless otherwise specified, a nucleotide sequence “encoding an amino acid sequence,” e.g., a polynucleotide “encoding” a chimeric polypeptide, defined below of the present disclosure, includes all nucleotide sequences that are degenerate versions of each other and that encode the same amino acid sequence.

### Polypeptides

Amino acids are referred to by either their commonly known three letter symbols or by the one-letter symbols recommended by the IUPAC-IUB Biochemical Nomenclature Commission. The amino acid residues are abbreviated as follows, where the abbreviations are shown in parentheses: alanine (Ala; A), asparagine (Asn; N), aspartic acid (Asp; D), arginine (Arg; R), cysteine (Cys; C), glutamic acid (Glu; E), glutamine (Gln; Q), glycine (Gly; G), histidine (His; H), isoleucine (Ile; I), leucine (Leu; L), lysine (Lys; K), methionine (Met; M), phenylalanine (Phe; F), proline (Pro; P), serine (Ser; S), threonine (Thr; T), tryptophan (Trp; W), tyrosine (Tyr; Y), and valine (Val; V).

Amino acid sequences are written left to right in amino to carboxy orientation.

In some embodiments of the disclosure, “Polypeptide” may refer to a sequence of amino acid subunits. In some embodiments, a “peptide” can be less than or equal to 50 amino acids long, e.g., about 5, 10, 15, 20, 25, 30, 35, 40, 45, or 50 amino acidslong. “Polypeptide,” refers to proteins, polypeptides, and peptides of any length, size, structure, or function. “Polypeptide,” “peptide,” and “protein” are used interchangeably to refer to polymers of amino acids of any length.

Polypeptides of the disclosure may comprise naturally or synthetically created or modified amino acids, for example, disulfide bond formation, glycosylation, lipidation, acetylation, phosphorylation, or any other manipulation or modification, such as conjugation with a labeling component. Also included within the definition are, for example, polypeptides in which one or more amino acid residues are artificial chemical analogs of a corresponding naturally occurring amino acid (including, for example, synthetic amino acids such as homocysteine, ornithine, p-acetylphenylalanine, D-amino acids, and creatine), as well as other modifications known in the art. Polypeptides also include gene products, homologs, orthologs, paralogs, fragments and other equivalents, variants, and analogs of the foregoing. A polypeptide may comprise a single polypeptide or can be a multi-molecular complex such as a dimer, trimer or tetramer. Polypeptides of the disclosure may comprise single-chain or multi-chain polypeptides. Most commonly disulfide linkages are found in multi-chain polypeptides.

The polypeptides of the disclosure may comprise L-amino acids+glycine, D-amino acids+glycine (which are resistant to L-amino acid-specific proteases in vivo), or a combination of D- and L-amino acids+glycine. Polypeptides described may be chemically synthesized or recombinantly expressed.

The polypeptides of the disclosure can include additional residues at the N-terminus, C-terminus, internal to the polypeptide, or a combination thereof; these additional residues are not included in determining the percent identity of the polypeptides of the disclosure relative to the reference polypeptide. Such residues may be any residues suitable for an intended use, including but not limited to tags.

In some embodiments of the disclosure, “chimeric polypeptide” may refer to any polypeptide comprised of a first amino acid sequence derived from a first source, bonded, covalently or noncovalently, to a second amino acid sequence derived from a second source, wherein the first and second source are not the same. In some embodiments, a first source and a second source that are not the same can include two different biological entities, or two different proteins from the same biological entity, or a biological entity and a non-biological entity. A chimeric protein can include for example, a protein derived from at least 2 different biological sources. In some embodiments, the chimeric polypeptide may include sequences from similar proteins derived from two distinct species. In some embodiments, the chimeric polypeptide may include sequences from dissimilar proteins derived from the same species. A biological source can include any non-synthetically produced nucleic acid or amino acid sequence (e.g. a genomic or cDNA sequence, a plasmid or viral vector, a native virion or a mutant or analog of any of the above). A synthetic source can include a protein or nucleic acid sequence produced chemically and not by a biological system (e.g. solid phase synthesis of amino acid sequences). A chimeric protein can also include a protein derived from at least 2 different synthetic sources or a protein derived from at least one biological source and at least one synthetic source. A chimeric protein may also comprise a first amino acid sequence derived from a first source, covalently or noncovalently linked to a nucleic acid, derived from any source or a small organic or inorganic molecule derived from any source. The chimeric protein can comprise a linker molecule between the first and second amino acid sequence or between the first amino acid sequence and the nucleic acid, or between the first amino acid sequence and the small organic or inorganic molecule.

In some embodiments of the disclosure, a “fragment” of a polypeptide, or a “truncated polypeptide” may refers to an amino acid sequence of a polypeptide that is shorter than the sequence of a reference polypeptide (which may be a naturally-occurring sequence). In comparison to the reference polypeptide, the fragment may comprise an N- and/or C-terminal deletion. In comparison to the reference polypeptide, the fragment may comprise a deletion of any part of the sequence, whether or not the deletion is contiguous. A polypeptide in which internal amino acids have been deleted with respect to the naturally occurring sequence is also considered a fragment. The various polypeptide components of the disclosure may be provided as fragments or truncated versions of a reference protein.

In some embodiments of the disclosure, a “functional fragment” may refer to a polypeptide fragment that retains a function of the polypeptide. In some embodiments, a functional fragment of a bioactive peptide (e.g., an enzyme), retains the ability to catalyze a biological action because the functional fragment comprises a catalytic domain of the enzyme. Polypeptides of the disclosure may be provided as functional fragments or truncated versions.

In some embodiments of the disclosure, “amino acid substitution” may refer to replacing an amino acid residue present in a parent or reference sequence with another amino acid residue. In some embodiments, the parent or reference sequence comprises a wildtype sequence. An amino acid can be substituted, for example, via chemical peptide synthesis or through recombinant methods known in the art. For example, substituting an amino acid residue with an alternative amino acid residue is conducted by substituting the codon encoding the first amino acid with a codon encoding the second amino acid. Polypeptides of the disclosure may be provided with one or more amino acid substitutions.

In some embodiments of the disclosure, a “conservative amino acid substitution” is one in which one amino acid residue is replaced with an amino acid residue having a chemically similar side chain. Families of amino acid residues having similar side chains have been defined in the art, including acidic side chains (e.g., aspartic acid, glutamic acid), basic side chains (e.g., lysine, arginine, histidine), uncharged polar side chains (e.g., glycine, asparagine, glutamine, serine, threonine, tyrosine, cysteine), nonpolar side chains (e.g., alanine, valine, leucine, isoleucine, proline, phenylalanine, methionine, tryptophan), beta-branched side chains (e.g., threonine, valine, isoleucine) and aromatic side chains (e.g., tyrosine, phenylalanine, tryptophan, histidine). Thus, if an amino acid in a polypeptide is replaced with another amino acid from the same side chain family, the substitution is considered to be conservative. In some embodiments, a string of amino acids can be conservatively replaced with a chemically similar string that differs in order and/or composition of side chain family members. The various polypeptide components of the disclosure may be provided with conservative amino acid substitutions.

In some embodiments of the disclosure, non-conservative amino acid substitutions include those in which (i) a residue having an electropositive side chain (e.g., Arg, His or Lys) is substituted for, or by, an electronegative residue (e.g., Glu or Asp), (ii) a hydrophilic residue (e.g., Ser or Thr) is substituted for, or by, a hydrophobic residue (e.g., Ala, Leu, Ile, Phe or Val), (iii) a cysteine or proline is substituted for, or by, any other residue, or (iv) a residue having a bulky hydrophobic or aromatic side chain (e.g., Val, His, Ile or Trp) is substituted for, or by, one having a smaller side chain (e.g., Ala or Ser) or no side chain (e.g., Gly). The various polypeptide components of the disclosure may be provided with non-conservative amino acid substitutions. The likelihood that one of the foregoing non-conservative substitutions can alter functional properties of the protein is also correlated to the position of the substitution with respect to functionally important regions of the protein: some non-conservative substitutions can accordingly have little or no effect on biological properties. The various polypeptide components of the disclosure may be provided with non-conservative amino acid substitutions that do not significantly alter the functionality of the altered components.

### Sequence Analyses

In some embodiments of the disclosure, “identity” refers to the overall monomer conservation between polymeric molecules, e.g., between polypeptide molecules or polynucleotide molecules. “Identical” without any additional qualifiers, e.g., protein A is identical to protein B, implies the sequences are 100% identical (100% sequence identity). Describing two sequences as, e.g., “70% identical,” is equivalent to describing them as having, e.g., “70% sequence identity.”

When a position in the first sequence is occupied by the same amino acid as the corresponding position in the second sequence, then the molecules are identical at that position. The percent identity between the two sequences is a function of the number of identical positions shared by the sequences, taking into account the number of gaps, and the length of each gap, which needs to be introduced for optimal alignment of the two sequences. The comparison of sequences and determination of percent identity between two sequences can be accomplished using a mathematical algorithm.

In certain embodiments, the percentage identity (% ID) of a first amino acid (or nucleic acid) sequence to a second amino acid (or nucleic acid) sequence is calculated as % ID=100 (Y/Z), where Y is the number of amino acid (or nucleobase) residues scored as identical matches in the alignment of the first and second sequences (as aligned by visual inspection or a particular sequence alignment program) and Z is the total number of residues in the second sequence. If the length of a first sequence is longer than the second sequence, the percent identity of the first sequence to the second sequence will be higher than the percent identity of the second sequence to the first sequence.

Calculation of the percent identity of two polypeptide sequences, for example, can be performed by aligning the two sequences for optimal comparison purposes. For example, gaps can be introduced in one or both of a first and a second polypeptide sequences for optimal alignment and non-identical sequences can be disregarded for comparison purposes. In certain embodiments, the length of a sequence aligned for comparison purposes is at least 30%, at least 40%, at least 50%, at least 60%, at least 70%, at least 80%, at least 90%, at least 95%, or 100% of the length of the reference sequence. The amino acids at corresponding amino acid positions are then compared.

Generation of a sequence alignment for the calculation of a percent sequence identity is not limited to binary sequence-sequence comparisons exclusively driven by primary sequence data. It will also be appreciated that sequence alignments can be generated by integrating sequence data with data from heterogeneous sources such as structural data (e.g., crystallographic protein structures), functional data (e.g., location of mutations), or phylogenetic data. A suitable program that integrates heterogeneous data to generate a multiple sequence alignment is T-Coffee, available at www.tcoffee.org, and alternatively available, e.g., from the European Bioinformatics Institute (EBI) at website ebi.ac.uk/Tools/psa. It will also be appreciated that the final alignment used to calculate percent sequence identity can be curated either automatically or manually.

Suitable software programs are available from various sources, and for alignment of both protein and nucleotide sequences. One suitable program to determine percent sequence identity is b12seq, part of the BLAST suite of programs available from the U.S. government's National Center for Biotechnology Information BLAST website (blast.ncbi.nlm.nih.gov). B12seq performs a comparison between two sequences using either the BLASTN or BLASTP algorithm. BLASTN is used to compare nucleic acid sequences, while BLASTP is used to compare amino acid sequences. Other suitable programs are, e.g., Needle, Stretcher, Water, or Matcher, part of the EMBOSS suite of bioinformatics programs and also available from the EBI. Sequence alignments can be conducted using methods known in the art such as MAFFT, Clustal (ClustalW, Clustal X or Clustal Omega), MUSCLE, etc. Different regions within a single polynucleotide or polypeptide target sequence that aligns with a polynucleotide or polypeptide reference sequence can each have their own percent sequence identity. It is noted that the percent sequence identity value is rounded to the nearest tenth. For example, values from 80.11 to 80.14 are rounded down to 80.1, while values from 80.15 to 80.19 are rounded up to 80.2. It also is noted that the length value will always be an integer.

In some embodiments of the disclosure, “non-naturally occurring” means a polypeptide or a polynucleotide sequence that does not exist in nature. In some embodiments, the non-naturally occurring sequence does not exist in nature because the sequence is altered relative to a naturally occurring sequence. In some embodiments, the non-naturally occurring sequence does not exist in nature because it is a combination of two known, naturally-occurring, sequences (e.g., chimeric polypeptide) that do not occur together in nature. In some embodiments, a non-naturally occurring polypeptide is a chimeric polypeptide. In some embodiments, a polypeptide or a polynucleotide is not naturally occurring because the sequence contains a portion (e.g., a fragment) that cannot be found in nature, i.e., a novel sequence. Any of the polynucleotides described herein may be provided as non-naturally occurring sequences, e.g., having sequences which are altered relative to native sequences or provided as polynucleotides which are linked to other polynucleotides in a manner that does not exist in nature. Any of the polypeptides described herein may be provided as non-naturally occurring sequences, e.g., having sequences which are altered relative to native sequences or provided as polypeptides which are linked to other polypeptides in a manner that does not exist in nature.

### Therapeutic Methods

In some embodiments of the disclosure, the term “therapeutically effective” may refer to imparting a beneficial effect on the recipient, e.g., providing some alleviation, mitigation, or decrease in at least one clinical symptom in the subject. Therapeutic effects of the disclosure need not be complete or curative, as long as some benefit is provided to the subject. For example, a therapeutic regimen that incorporates the polynucleotides, gene therapy vectors or cells of the disclosure with the small molecules of the disclosure may be structured such that the regimen is therapeutically effective as a whole.

In some embodiments of the disclosure, the term “therapeutically effective amount” refers to a dose or an amount of a nucleic acid, vector, polypeptide, composition, pharmaceutical composition or cell of the disclosure sufficient to impart a therapeutically effective benefit on the recipient. For example, polynucleotides, gene therapy vectors or cells of the disclosure may be administered in a therapeutically effective amount. A subject who has been administered polynucleotides, gene therapy vectors or cells of the disclosure may subsequently be administered a therapeutically effective amount of a small molecule of the disclosure, i.e., an amount sufficient to impart a beneficial effect on the recipient given the previous administration of polynucleotides, gene therapy vectors or cells.

### Cell Therapy

In some embodiments of the disclosure, the term “stem cell” may refer to an undifferentiated or partially differentiated cell that can differentiate into various types of cells and proliferate indefinitely to produce more of the same stem cell.

In some embodiments of the disclosure, the term “Pluripotent stem cell” (PSC) may refer to a cell that can maintain an undifferentiated state indefinitely and can differentiate into most, if not all cells of the body.

In some embodiments of the disclosure, the term “Induced pluripotent stem cell” (iPS or iPSC) may refer to a pluripotent stem cell that can be generated directly from a somatic cell. This includes, but is not limited to, specialized cells such as skin or blood cells derived from an adult.

In some embodiments of the disclosure, the term “multipotent” may refer to a cell that can develop into more than one cell type but is more limited than a pluripotent cell. For example, adult stem cells and cord blood stem cells may be considered as multipotent.

In some embodiments of the disclosure, the term “hematopoietic cell” may refer to a cell that arises from a hematopoietic stem cell (HSC). Hematopoietic cells of the disclosure include, but is not limited to, myeloid progenitor cells, lymphoid progenitor cells, megakaryocytes, erythrocytes, mast cells, myeloblasts, basophils, neutrophils, eosinophils, macrophages, thrombocytes, monocytes, natural killer cells, T lymphocytes, B lymphocytes and plasma cells.

In some embodiments of the disclosure, the term “T-lymphocyte” or “T-cell” may refer to a hematopoietic cell that normally develops in the thymus. T-lymphocytes or T-cells include, but are not limited to, natural killer T cells, regulatory T cells, helper T cells, cytotoxic T cells, memory T cells, gamma delta T cells, and mucosal invariant T cells.

In some embodiments of the disclosure, the term “mesenchyme” may refer to a type of animal tissue comprising loose cells embedded in a mesh of proteins and fluid, i.e., the extracellular matrix. Mesenchyme directly gives rise to most of the body's connective tissues including bones, cartilage, lymphatic system, and circulatory system.

In some embodiments of the disclosure, the term “mesenchymal cell” may refer to a cell that is derived from a mesenchymal tissue. In some embodiments, cells of the disclosure may be mesenchymal cells.

In some embodiments of the disclosure, the term “mesenchymal stromal cell” (MSC) may refer to a spindle shaped plastic-adherent cell isolated from bone marrow, adipose, and other tissue sources, with multipotent differentiation capacity in vitro. For example, a mesenchymal stromal cell can differentiate into osteoblasts (bone cells), chondrocytes (cartilage cells), myocytes (muscle cells), and adipocytes (fat cells which give rise to marrow adipose tissue). The term mesenchymal stromal cell is suggested in the scientific literature to replace the term “mesenchymal stem cell”. In some cases, cells of the disclosure may be mesenchymal stromal cells.

In some embodiments of the disclosure, an “autologous cell” is a cell obtained from the same individual to whom it may be administered as a therapy (the cell is autologous to the subject). Autologous cells of the disclosure include, but are not limited to, hematopoietic cells and stem cells, such as hematopoietic stem cells.

In some embodiments of the disclosure, an allogeneic cell is a cell obtained from an individual who is not the intended recipient of the cell as a therapy (the cell is allogeneic to the subject). Allogeneic cells of the disclosure may be selected from immunologically compatible donors with respect to the subject of the methods of the disclosure. Allogeneic cells of the disclosure may be modified to produce “universal” allogeneic cells, suitable for administration to any subject without unintended immunogenicity. Allogeneic cells of the disclosure include, but are not limited to, hematopoietic cells and stem cells, such as hematopoietic stem cells.

In some embodiments of the disclosure, the term “Transfect” or “transform” or “transduce” may refer to a process by which exogenous nucleic acid is transferred or introduced into a host cell. In some embodiments, a “transfected” or “transformed” or “transduced” cell is one which has been transfected, transformed or transduced with exogenous nucleic acid or progeny of the cell.

In some embodiments of the disclosure, the term “Cell therapy” may refer to the provision or delivery of cells into a recipient for therapeutic purposes.

### Formulations

In some embodiments of the disclosure, “pharmaceutically acceptable” refers to compounds, materials, compositions, and/or dosage forms which are, within the scope of sound medical judgment, suitable for use in contact with the tissues of human beings and animals without excessive toxicity, irritation, allergic response, or other problem or complication commensurate with a reasonable benefit/risk ratio. For example, the small molecules, polynucleotides, polypeptides, gene therapy vectors or cells of the disclosure may be administered as part of a composition together with other pharmaceutically acceptable components, including pharmaceutically acceptable carriers.

In some embodiments of the disclosure, the term “pharmaceutically acceptable salts” refers to derivatives of the small molecules of the disclosure wherein the specified compound is converted to an acid or base salt thereof. Such pharmaceutically acceptable salts include, but are not limited to, mineral or organic acid salts of basic residues such as amines; alkali or organic salts of acidic residues such as carboxylic acids; and the like. The pharmaceutically acceptable salts include the conventional non-toxic salts or the quaternary ammonium salts of the parent compound formed, for example, from non-toxic inorganic or organic acids. For example, such conventional non-toxic salts include those derived from inorganic acids such as hydrochloric, hydrobromic, sulfuric, sulfamic, phosphoric, nitric and the like; and the salts prepared from organic acids such as acetic, propionic, succinic, glycolic, stearic, lactic, malic, tartaric, citric, ascorbic, pamoic, maleic, hydroxymaleic, phenylacetic, glutamic, benzoic, salicylic, sulfanilic, 2-acetoxybenzoic, fumaric, toluensulfonic, methanesulfonic, ethanedisulfonic, oxalic, isethionic, and the like. For example, the small molecules of the disclosure may be provided as pharmaceutically acceptable salts.

In some embodiments of the disclosure, the term “excipients” refer to pharmacologically inert ingredients that are not active in the body. See, for example, Hancock, B. C., Moss, G. P., &amp; Goldfarb, D. J. (2020). Handbook of pharmaceutical excipients. London: Pharmaceutical Press, the entire disclosure of which is incorporated herein by reference. The small molecules of the disclosure may be mixed with pharmaceutically acceptable carriers, diluents, adjuvants, excipients, or vehicles, such as preserving agents, fillers, polymers, disintegrating agents, glidants, wetting agents, emulsifying agents, suspending agents, sweetening agents, flavoring agents, perfuming agents, lubricating agents, acidifying agents, and dispensing agents, depending on the nature of the mode of administration and dosage forms. Such ingredients, including pharmaceutically acceptable carriers and excipients that may be used to formulate oral dosage forms. Pharmaceutically acceptable carriers include water, ethanol, polyols, vegetable oils, fats, waxes polymers, including gel forming and non-gel forming polymers, and suitable mixtures thereof. Examples of excipients include starch, pregelatinized starch, Avicel, lactose, milk sugar, sodium citrate, calcium carbonate, dicalcium phosphate, and lake blend. Examples of disintegrating agents include starch, alginic acids, and certain complex silicates. Examples of lubricants include magnesium stearate, sodium lauryl sulfate, talc, as well as high molecular weight polyethylene glycols. For example, the small molecules, polynucleotides, gene therapy vectors or cells of the disclosure may be provided and administered in compositions that include pharmaceutically acceptable excipients.

### Sequences

TABLE 16
Exemplary Consensus Concatemer Sequences

| Gene | Consensus Motif |
|---|---|
| JUNB | RATGAWTCAT |
| JUND | NNTGAWTCATSNNNRRANT |
| BATF | TGAWTCATSNYNRRA |
| FOS | GATGAWTCAYNNN |
| BATF | DATGAWTCAT |
| ATF3 | DATGAWTCATHN |
| NFE2L1 | ATGACTCAY |
| JUNB | NNRTGAWTCATNNNN |
| FRA2 | GGATGACTCATC |
| IRF4 | NNWNNNGAANTGANSWSN |
| FOSL2 | NATGAWTCABNN |
| FOS | NDATGAWTCAYN |
| ZSCAN5C | GNTTTRTTNACTCAC |
| ZBTB37 | RTCGGCYAANSCGGCAN |
| ZNF442 | ACTCTTTCTCTGCTGCAAYACCTGCTGTY |
| ZBTB20 | NMYATACRTINN |
| SMAD3 | YGTCTAGACA |
| FRA1 | NNATGAWTCATH |
| AICE | GAAATGAGTCA |
| JUN-AP1 | GATGACTCATCN |
| JDP2 | ATGASTCAT |
| HOXD11 | RTCGTAAAAN |
| FOSL2 | NRTGASTCATCCNN |
| ZBTB7C | NCGACCACCGNN |
| POU1F1 | NNNATGMATASTTAATN |
| GRHL2 | NNNAACTGGTTTNNN |
| ZBTB7B | NCGACCACCGAA |
| NR2E3 | AAAGTCAAAAGTCA |
| HOXC9 | GTCGTAAAAN |
| TBX3 | NAGGTGTSAN |
| ZNF266 | NNNRCTCACAGGYCC |
| ZNF669 | GTCATCR |
| HOXC11 | NTTTWACGACN |
| HOXA10 | RGTCGTAAAAN |
| FOSL1 | NGGTGAWTCAT |
| ZNF587 | CANNTGGCGCCCAACATKGGNCNN |
| ATF1 | TKRCGTCAYNN |
| ZNF652 | TTAACCCTTTVNKKN |
| ATF2 | ATGACGTCAY |
| ZNF440 | ARTKGTTCTGCTNNN |
| PKNOX2 | TGACAGGTGTCA |
| SMAD5 | YGTCTAGACA |
| RFX1 | GGTTGCCATGGCAA |
| HOXB9 | GTCGTAAAAN |
| ZNF433 | ACYAWTGTRRTMATT |
| PKNOX1 | TGACAGWTGTCA |
| PBX2 | NTGATTGACAGN |
| ATF1 | GATGACGTCA |
| MYCN | NNCCACGTGGNN |
| SOX7 | AACAATRTNCAKTGTT |
| ZBTB22 | NKCACTAYNRTAGTGMN |
| BACH2 | NNTGACTCAGCANN |
| NFKB2 | NGGGGASTCCCCN |
| OSR2 | NCNGCTNCTGN |
| BACH2 | TGCTGAGTCA |
| TGIF2LY | TGACAGCTGTCA |
| DUX4 | TAAYYYAATCA |
| MAFK | TGCTGAWTCAGCA |
| VDR | GRGTTCATYGRGTTCA |
| IRF:BATF | CTTTCANTATGACTV |
| GLI3 | GACCACCCANG |
| JUN | CATNCCSNNNNGRTGAWKYM |
| CREB1 | NNTGACGTCANN |
| PBX3 | CCTGTCAATCAN |
| GLI1 | GACCACCCAMG |
| MAFG | NSSNTGCTGASTCAGCANSSN |
| MAFA | TGCTGACTCA |
| ATF7 | NGRTGACGTCAY |
| HOXA11 | GGTCGTAAAAN |
| PRDM14 | AGGTCTCTAACC |
| RXRA | NRRGGTCA |
| ZNF350 | CNGCYYTTTTSTNACCNNNCN |
| NRF | GTGCGCATGCGC |
| CREB3L4 | NRTGACGTCAYN |
| NF-E2 | GATGACTCAGCA |
| MYB | GGCVGTTR |
| ZBTB43 | NGTGCCNNNNNNNYAGCACN |
| NFE2L2 | ATGACTCAGCANSS |
| GATA:SCL | CGGCTGCNGNNNNCAGATAA |
| PPARD | NRRGGTCAAAGGTCAN |
| IRF4 | ACTGAAACCA |
| SPDEF | AMCCGGATGTS |
| BZIP:IRF | NAGTTTCABTHTGACTNS |
| ISL2 | YTAAKTGC |
| TGIF2 | TGTCANYT |
| KLF5 | GCCCCRCCCH |
| RELB | NNATTCCCCNN |
| AMYB | TGGCAGTTGG |
| KLF10 | GGGGGTGTGTCC |
| MAFF | TGCTGACTCAGCANS |
| ELF1 | NNNASCCGGAAGYGW |
| NR2F1 | RRGGTCAAAGGTCAN |
| ESR1 | NNNCCAAGGTCANN |
| ZNF736 | AAAARCCCCAGA |
| COUP-TFII | AGRGGTCA |
| ZNF26 | TGATGAC |
| TCF7 | AWATCAAAG |
| BACH1 | ASSNTGCTGAGTCAT |
| POU3F3 | SSTATGCSAATTW |
| GATA3 | NSGATARNNATCSWN |
| TFAP4 | ASCAGCTGAT |
| NFE2L2 | ASSSTGCTGAGTCAT |
| BCL11A | NTTTCAWTTCCYCNT |
| ANHX | TTGACASG |
| ZNF467 | TGGGGAAGGGCM |
| NKX2-3 | NNNAAGTGWN |
| GATA | NAGATSNBNATCTNN |
| ZNF282 | CTCCCW |
| ZNF561 | TTTCTGC |
| MAFB | SNTGCTGAWTCAGCASWTTY |
| GLI2 | GACCACCCANG |
| IRF6 | RGTSTCGNNNNNNYGASACT |
| MAFB | NNTGCTGAWTCAGCANSS |
| ATOH1 | AACAGCTGTC |
| NRF2 | HTGCTGAGTCAT |
| ASCL1 | NNAGCAGCTGWNN |
| ZIM3 | NNTRGNTTTCTGTINNYN |
| SALLA | CCCSCCCNNC |
| NFE2 | NATGACTCAGCANSTNN |
| STATI | NYCNTTTCCNGGAANNCNNNN |
| ZKSCAN5 | TCACCTCCN |
| LYL1 | NNGRNARCAGMTGN |
| TIISRE | ACTTTCGTTTCT |
| ZKSCAN7 | NYAAACTGTNRAGYGCN |
| KLF14 | GGCCMCGCCCMCYN |
| GATA5 | WGATAANN |
| ZNF439 | CAGTCACCYTCSGG |
| CREB3 | NNRTGACGTCAYCM |
| EAR2 | NRBCARRGGTCA |
| FOXP1 | NNGTAAACAGNN |
| STAT3 | CNTTTMCYGGAARTWANNNN |
| NR1H4 | NNGGTCATINN |
| PATZ1 | CCCCKCCC |
| FOXK2 | WCHTGTTTACAT |
| OVOL2 | NNSCCGTTANNNN |
| RXR | TAGGGCAAAGGTCA |
| AP4 | NAHCAGCTGD |
| MOUSE_ | ACTYKNATTCGTGNTACTTC |
| RECOMBINATION_ | |
| HOTSPOT | |
| GATA1 | CAGATAAGGN |
| ZNF141 | NNNNNNNNGGKGGGRGCGTCCCCC |
| HIC1 | TGCCAGCB |
| ZNF821 | NTGTCYGTCTGTCCN |
| ZNF174 | NGNCRRTCACTYGCCN |
| MAF | NGTCAGCAN |
| MYOD | AGCAGCTGCTGC |
| CHR | CGGTTTCAAA |
| C-JUN-CRE | ATGACGTCATCC |
| GATA2 | NNSGATAAGNN |
| STAT6 | NTTCCWWAGAA |
| ELK1 | ACCGGAAGTN |
| MSX1 | TAATTGWNNTTTAATTGN |
| HAND2 | TGACANARRCCAGRC |
| MSX2 | TAATTGWSSTTTAATTGC |
| PBX3 | NNNNGCCAATWRNNN |
| ZNF12 | YNYTYWTYNCNGCNGCNW |
| KLF1 | VDGGGYGGGGCY |
| GATA2 | NCCTTATCTC |
| SP5 | RGKGGGCGGAGC |
| RUNX2 | NRACCGCAAACCGCAN |
| MYB | NNCAACTGNN |
| ETS:RUNX | ACAGGATGTGGT |
| BMYB | NHAACBGYYV |
| ESRRB | TCAAGGTCASN |
| ZNF681 | YCNNCGTCCTTN |
| HNF4A | NRGNNCAAAGKYCAN |
| NRL | GTCAGCANNNN |
| ZNF77 | ACTYCAC |
| AP-2ALPHA | ATGCCCTGAGGC |
| NKX2-5 | NTYAAGTGGN |
| ZNF223 | YGCCMYCTDWTGGC |
| RFX6 | TGTTKCCTAGCAACM |
| ZSCAN22 | WMCAGTCSGAKGGAGGAGGC |
| ZNF740 | NCCCCCCCAC |
| NF1:FOXA1 | NNTGTTTATTTTGGCA |
| GATA1 | NWAGATAAGNN |
| BARX2 | NTAATKRTTSSN |
| TCF3 | ACATCAAAGG |
| ZKSCAN3 | GGCTAGCCC |
| STAT5B | NNTTCYMRGAAN |
| ZNF75D | GTGGGAAAGCCT |
| WT1 | CYCCNCCYCCTCCCCCNCCY |
| MAZ | GGGGGGGG |
| MEIS1 | VGCTGWCAVB |
| ZNF431 | NNGNCTTCYAGCCYN |
| ZNF708 | NNGCTGTACCTNCYNNNNNNN |
| STAT2 | NTNAGTTTCRNTTTC |
| NFIX | YTGGCNNNNTGCCAA |
| FOXL1 | RTAAACA |
| IRF2 | WGAAAGYGAAASCNSSNC |
| ZNF398 | CTSYYTCCCTCCCT |
| CREB5 | VVATGACGTCAT |
| BARX1 | TAATNGNTSTTTAATNG |
| PRDM9 | ADGGYAGYAGCATCT |
| TAL1 | NCTTATCTWNNNNNNWCAG |
| IRF1 | AAANNGAAAGTGAAAWTRNNN |
| USF1 | GGTCACGTGA |
| FXR | AGGTCANTGACCTN |
| ZBTB2 | NTTTCCGGTAAN |
| ZNF304 | CCCCCKCCCCRSTCWRGCCYNNNN |
| OLIG3 | AMCATATGNY |
| KLF9 | NNCCACACCCACN |
| ZNF232 | CTTAATCTACRTTTAACAY |
| TFEB | RTCACGTGAY |
| ZNF468 | CRCCCCCTCCCN |
| ZNF684 | ACAGTCCACCCCTT |
| FEV | ACCGGAAGTN |
| ASCL1 | NNVVCAGCTGBN |
| MLX | ATCACGTGAY |
| ALX3 | NNTAATTRNN |
| UNKNOWN-ESC- | CACAGCAGGGGG |
| ELEMENT | |
| ZNF302 | AGTTGAGTGACTGYRGTT |
| ZNF263 | CYCCYCCYCNNCCTCCTCCYY |
| ZNF649 | TTATAT |
| ETV4 | ACCGGAAGTN |
| KLF12 | CCNNGCCCCGCCCCN |
| MYF6 | AACARCTGTT |
| OVOL1 | NNSACCGTTATNYN |
| FOXC2 | NGTMAATATTKACN |
| NEUROG2 | ACCATCTGTT |
| INSM1 | YGCCCCCTGRCA |
| ZNF695 | TTCCTCY |
| ZNF41 | CCTCATGGTGYCYTSYTCCCTTGTG |
| ZNF563 | NTCCNNNCNGGCARCTGY |
| GATA | NNNNNBAGATASYATCTVHN |
| ZNF200 | AYTTCC |
| BACH1 | GTCACGTG |
| ZNF250 | YNNTGCANTTTTTNN |
| IRF5 | CCGAAACCGAAACY |
| ATF3 | GGTCACGTGRW |
| RBPJ1 | HTTTCCCAWG |
| ZNF285 | GCCAATTYCYRTGGTGTAAAT |
| IRF3 | AGTTTCAKTTTC |
| SCL | ANCAGCTG |
| KLF14 | RGKGGGCGKGGC |
| TGIF1 | YTGWCADY |
| GATA4 | WGATAANN |
| ELK4 | CCGGAAGYGN |
| KLF6 | MKGGGYGTGGCC |
| MITF | RTCATGTGAC |
| ZNF140 | CAATTCCGCTCMNNNNNNNNNNNN |
| ELF2 | NANSCGGAAGTN |
| PPARA | VNAGGKCAAAGGTCA |
| TFEC | RNCACRTGAY |
| RORC | AAYTAGGTCA |
| GATA4 | NBWGATAAGR |
| COUP-TFII | GKBCARAGGTCA |
| ZNF662 | RKCTAGCCCNNKCCTACC |
| RORA | AASCTAGGTCARDNN |
| PGR | NGNACANNNYGTNCY |
| TBX20 | GGTGYTGACAGW |
| ZSCAN31 | NNNTTAATTGNTTKTN |
| ZNF707 | ACCCCACTCCTGGTM |
| IRF8 | NCGAAACCGAAACY |
| NFATC3 | WNTTTCCRTN |
| ZBTB45 | NMTATAGGNGN |
| ATF2 | NRRTGACGTCAT |
| ZNF766 | GGTTTMT |
| FOXP2 | NWGTAAACARN |
| GABPA | NRCCGGAAGTG |
| ASCL2 | NRCAGCTGYN |
| ELF3 | ANCAGGAAGT |
| SPDEF | ACATCCTGGT |
| EHF | AVCAGGAAGT |
| PRDM10 | TGGTACATTCCA |
| REL | SGGRNWTTCC |
| ZNF605 | NNNYNKGGYNTCTNNNNCCCY |
| ZNF419 | ASTMCAGCCCTAKACTCTCCY |
| GATA3 | AGATWINDNNDWAGATAAWN |
| PPARE | TGACCTTTGCCCCA |
| MEF2D | GCTATTTTTAGC |
| ETS | AACCGGAAGT |
| MAZ | WCCCCKCCC |
| GCM2 | NATGCGGGTN |
| GATA6 | YCTTATCTBN |
| KLF17 | MMCCACGCACCCMTY |
| ZNF341 | GCTWTTCCNNCNNCCNCCCNN |
| ERF | ACCGGAAGTR |
| ETV2 | AACCGGAAATR |
| SP9 | NCCACGCCCMCN |
| ELF5 | ANSMGGAAGTN |
| ELF1 | AACCGGAAGT |
| ELF5 | ACVAGGAAGT |
| NFAT:AP1 | GAATGGAAAAAATGAGTCAT |
| MEIS3 | NTGACAGN |
| RBPJ:EBOX | GGGRAARRGRMCAGMTG |
| SP8 | RCCACGCCCMCY |
| NEUROD1 | GCCATCTGTT |
| ZNF891 | NNNGNNCNWNGGCTTCYAGCC |
| RORA | AWNNAGGTCA |
| TBOX:SMAD | AGGTGHCAGACA |
| ERRG | GTGACCTTGRVN |
| MAFK | GCTGASTCAGCA |
| ZNF157 | TRTAYTGCSNNCCYTNCCANGSCM |
| AP-2GAMMA | WCCTWAGGWCAS |
| IRX3 | SACATGNSSSSNCATGTS |
| ZNF71 | NAGTITCRTTTTCY |
| IRF8 | GRAAWTGAAAWT |
| NFATC1 | ATTTTCCATT |
| PU.1:IRF8 | GGAAGTGAAAWT |
| ETS1-DISTAL | AACAGGAAGT |
| RXRG | GGTCAAAGGTCA |
| SP3 | NCCACGCCCMC |
| ETV2 | NNAYTTCCTGHN |
| ZFP82 | AGAATTAGTGAACTGGAAGAT |
| RORC | SAABTAGGTCAV |
| ISRE | AGTTTCAGTTTC |
| NFATC4 | WNTTTCCRYN |
| TLX? | CTGGCAGGCTGCCA |
| ELK4 | NRYTTCCGGY |
| IRF7 | NCGAAARYGAAANT |
| ELF4 | ACTTCCKGKT |
| ELK1 | HACTTCCGGY |
| REL | GGAAATTCCC |
| IRF9 | AACGAAACCGAAACT |
| IRF2 | GAAAWYGAAAWY |
| ESRRB | GTGACCTTGA |
| EWS:FLI1- | AACAGGAAAT |
| FUSION | |
| RUNX1 | AAACCACAAA |
| EWS:ERG-FUSION | ATTTCCTGTN |
| ETV4 | ACCGGAAGTG |
| REL | AGGGGATTTCCC |
| IRF1 | GAAAGTGAAAGT |
| KLF16 | GMCACGCCCCC |
| EGR4 | NNMCGCCCACGCANNN |
| RELA | NGGGRMTTTCCMNN |
| RUNX1 | CAAACCACAG |
| RUNX3 | AACCRCAA |
| RUNX2 | NWAACCACADNN |
| GABPA | AACCGGAAGT |
| NR2F2 | NNAAGGTCANN |
| NR4A1 | NAAAGGTCAN |
| RUNX1 | NWAACCACARN |
| ERG | ACAGGAAGTG |
| FLI1 | NRYTTCCGGH |
| PU.1 | AGAGGAAGTG |
| EGR1 | NNNNNNNNCCGCCCMCGCNN |
| RUNX1 | GCTGTGGTTT |
| ETV1 | AACCGGAAGT |
| ETS1 | ACAGGAAGTG |
| WT1 | MCTCCCMCRCAB |
| EGR3 | CKCCCCCNC |
| EGR2 | NGCGTGGGCGGR |
| EGR2 | CGCCCMCNC |
| EGR1 | TGCGTGGGYG |
| NUR77 | TGACCTTINCNT |

The identified consensus motifs of Table 16 comprise some variability within the identified sequence. In some embodiments, the identified consensus motifs of Table 16 may allow for one or more nucleotide substitutions within the identified consensus motifs. For example, within the consensus motifs of Table 16, “N” may allow for any nucleotide to be substituted at that position including A, G, C, T; “S” may allow for either G or C to be substituted at that position; “R” may allow for either A or G to be substituted at that position; and “W” may allow for A or T to be substituted at that position.

Table 18 below lists specific motifs that were identified that reduce the variability of the consensus motifs. Table 18 lists exemplary specific motifs that exist within the identified consensus motifs of Table 16. The transcription initiator and the regulators of the disclosure may comprise any one or more of the specific exemplary motifs of Table 16.

Lengthy table referenced here
US20250223330A1-20250710-T00001
Please refer to the end of the specification for access instructions.

Lengthy table referenced here
US20250223330A1-20250710-T00002
Please refer to the end of the specification for access instructions.

Lengthy table referenced here
US20250223330A1-20250710-T00003
Please refer to the end of the specification for access instructions.

### Definitions

In some embodiments of the disclosure, the term “subject” refers to any mammal, including without limitation, humans.

The terms “a”, “an” and “the” include their plural forms unless the context clearly dictates otherwise.

The term “and” is used interchangeably with “or” unless expressly stated otherwise.

The term “And/or” is to be taken as specific disclosure of each of the two specified features or components with or without the other. Thus, “and/or” as used in a phrase such as “A and/or B,” includes “A and B,” “A or B,” “A” (alone), and “B” (alone). Likewise, “and/or,” as used in a phrase such as “A, B, and/or C,” is intended to encompass each of the following embodiments: A, B, and C; A, B, or C; A or C; A or B; B or C; A and C; A and B; B and C; A (alone); B (alone); and C (alone).

Numeric ranges are inclusive of the numbers defining the range. Where a range of values is stated, each intervening integer value, and each fraction thereof, between the recited upper and lower limits of that range is also specifically disclosed, as is each subrange between such values. The upper and lower limits of any range can independently be included in or excluded from the range, and each range where either, neither or both limits are included is also encompassed within the disclosure. Thus, ranges are understood to be shorthand for all of the values within the range, inclusive of the recited endpoints. For example, a range of 1 to 10 is understood to include any number, combination of numbers, or sub-range from the group consisting of 1, 2, 3, 4, 5, 6, 7, 8, 9, and 10.

Where a value is explicitly stated, it is to be understood that values which are about the same quantity or amount as the stated value are also within the scope of the disclosure. Where a combination is disclosed, each subcombination of the elements of that combination is also specifically disclosed and is within the scope of the disclosure. Conversely, where different elements or groups of elements are individually disclosed, combinations thereof are also disclosed. Where any element of a disclosure is disclosed as having a plurality of alternatives, examples of that disclosure in which each alternative is excluded singly or in any combination with the other alternatives are also hereby disclosed; more than one element of a disclosure can have such exclusions, and all combinations of elements having such exclusions are hereby disclosed.

Unless the context clearly requires otherwise, throughout the description and the claims, the words ‘comprise’, ‘comprising’, and the like are to be construed in an inclusive sense as opposed to an exclusive or exhaustive sense; that is to say, in the sense of “including, but not limited to”.

Singular or plural words also include the plural and singular number, respectively. Thus, for example, where the specification describes a gene of interest, the disclosure includes polynucleotides with a single gene of interest or multiple genes of interest.

Units, prefixes, and symbols are denoted in their Systeme International de Unites (SI) accepted form.

Headings are included herein for reference and to aid in locating the various sections. These headings are not intended to limit the scope of the concepts described with respect to the headings. Such concepts may have applicability throughout the present specification.

### EXAMPLES

### Example 1: General Methods

### ELISA Experimental Details

### Materials

| Item | Catalog# | Manufacturer |
|---|---|---|
| Strep-Tactin ®XT coated | 2-4101-001 | iba |
| microplate | | |
| mouse anti-STII | A01732 | Genscript |
| anti-mouse IR detection module | DM-010 | Protein Simple |
| CD25-Fc | 1020-RL-050 | R&D |
| IL12Rb2-Fc (control) | 1959-B2B-050 | R&D |
| Blocker ™ Casein in TBS | 37532 | Thermo |
| Protein GHRP | 18-161 | Millipore |
| 20X Wash Buffer-0.05% | 28352 | Thermo-Fisher |
| PBS-Tween (needs to be diluted | | |
| with dH20 from Milli Q) | | |
| Ultra Pure DNase/RNase-Free | 10977015 | Invitrogen-Thermo |
| Distilled Water | | |
| 1xPBS pH 7.2 | 20012043 | Gibco |
| TMB Ultra | 34028 | Thermo-Fisher |
| Stop Solution | 77316 | BioLegend |
| Recombinant human IL2 | 10453-IL-100 | R&D |
| Normocin | ant-nr-1 | Invivogen |
| Puromycin | ant-pr-1 | Invivogen |
| HEK-Blue CLR selection | hb-csm | Invivogen |
| Normocin | ant-nr-1 | Invivogen |
| Puromycin | ant-pr-1 | Invivogen |
| Blasticidin | ant-bl-05 | Invivogen |
| Hygromycin B Gold | ant-hg-1 | Invivogen |
| Zeocin | ant-zn-05 | Invivogen |
| HEK-Blue IL2R | hkb-il2 | Invivogen |
| HEK-Blue CD122/132 | hkb-il2bg | Invivogen |

Dissolve d50 micrograms (mg) of CD25-Fc (R&D 1020-RL-050) in 500 microliters (mL) of phosphate buffered saline (PBS). For this 100 mg/mL solution, freeze in 20 mL aliquots at −80° C.

Dissolved 50 mg of CD25-His (R&D 10305-RL-050) in 100 mL of PBS. For this 500 mg/mL solution, freeze in 10 mL aliquots at −80° C.

Captured STII-tagged Designed Cytokines from undiluted supernatant (100 mL/well) on streptactin plates for 1 hour at room temperature. Wash plate with 4×200 mL with PBS-T.

Prepared and added Primary antibody to plate: dilution buffer contains casein block and PBS-T (1:1). Added 2 mg/mL CD25-Fc in Casein Block+PBS-T (1:1) and diluted 100 mL of 100 mg/mL CD25-Fc in 5 milliliters (mL) of dilution buffer. Added 0.5 mg/mL CD25-Fc in Casein Block+PBS-T (1:1) and then diluted 25 mL of 100 mg/mL CD25-Fc in 5 mL of dilution buffer. Mixed scIL12-anti-PDL1-scFv-STII+IL12Rb2-Fc at the specified concentration in casein block+PBS-T (1:1). Incubated for 1 hr at room temperature.

Washed plate with 4×200 mL PBS-T.

Prepared and added secondary antibody to plate: Make Secondary Antibody composition by mixing Protein G and HRP (1:5000) in casein block+PBS-T (1:1). Incubate for 45 min at room temperature.

Washed plate with 4×250 mL PBS-T.

Added 75 mL of TMB-Ultra and incubated for approximately 5 min.

Added 75 mL of 2M HCl.

Measured absorbance at 450 nm (A450) on the plate reader using the ELISA program.

### HEK Blue—Experimental Details

| | Cat. | Stock | Volume | | Final | |
|---|---|---|---|---|---|---|
| Item | No. | Conc. | to Add | | Conc. | |
| DMEM (4.5 g/L | | x | 500 | mL | | |
| glucose, 2 mM | | | | | | |
| L-glutamine) | | | | | | |
| Heat Inactivated | | x | 50 | mL | 10% | |
| FBS | | | | | | |
| Penicillin/ | | 100X | 5 | mL | 100 U/mL penicillin + | |
| Streptomycin | | | | | 100 ug/mL streptomycin | |
| (100x) | | | | | | |
| Normocin | ant- | 50 | 1 | mL | 100 | ug/mL |
| | nr-1 | mg/mL | | | | |
| Puromycin | ant- | 10 | 50 | uL | 1 | ug/mL |
| | pr-1 | mg/mL | | | | |
| HEK-Blue CLR | hb- | 250X | 2 | mL | 1X | |
| selection | csm | | | | | |

| | Cat. | Stock | Volume | | Final | |
|---|---|---|---|---|---|---|
| Item | No. | Conc. | to Add | | Conc. | |
| DMEM (4.5 g/L | | | 500 | mL | | |
| glucose, 2 mM | | | | | | |
| L-glutamine) | | | | | | |
| Heat Inactivated | | x | 50 | mL | 10% | |
| FBS | | | | | | |
| Penicillin/ | | 100X | 5 | mL | 100 U/mL penicillin + | |
| Streptomycin | | | | | 100 ug/mL streptomycin | |
| (100x) | | | | | | |
| Normocin | ant- | 50 | 1 | mL | 100 | ug/mL |
| | nr-1 | mg/mL | | | | |
| Puromycin | ant- | 10 | 50 | uL | 1 | ug/mL |
| | pr-1 | mg/mL | | | | |
| Blasticidin | ant- | 10 | 500 | uL | 10 | ug/mL |
| | bl-05 | mg/mL | | | | |
| Hygromycin B | ant- | 100 | 500 | uL | 100 | ug/mL |
| Gold | hg-1 | mg/mL | | | | |
| Zeocin | ant- | 100 | 500 | uL | 100/mL | |
| | zn-05 | mg/mL | | | | |

Detached cells using 10 mL PBS/flask, count cells, spin down 300xg 5 min, seed 1e6 cells into new flasks, resuspend cell pellet in DMEM+10% FBS to a density of 280K/mL, and plate 180 mL cells/well in a flat bottom 96 well plate.

Prepared dilutions of supernatants in a 96 well PCR plate:

Prepared dilution of supernatant for these plates. Added 120 mL/well of undiluted supernatant to a PCR plate

Transferred 20 mL of this to 180 mL of Expi293 media for a 1:10 dilution.

Transferred 40 mL of the 1:10 to 160 mL of Expi293 media for a 1:50 final dilution

Transferred 100 mL of the 1:50 into 100 mL of Expi293 for a 1:100 dilution

Transferred 100 mL of 1:100 into 100 mL of Expi293 for a 1:200 dilution

Transferred 100 mL of 1:200 into 100 mL of Expi293 for a 1:400 dilution

Prepared dilutions of Designed Cytokines: Prepared 100 mM Acetic Acid from glacial acetic acid (17.4M). Diluted 57.5 uL of glacial acetic acid into 10 mL of ultrapure water. Sterile filtered. Dissolved 100 mg of IL-2 (R&D 10453-IL-100) in 200 mL of 100 mM Acetic Acid to make a 500 mg/mL solution.

Used Designed Cytokines over a range from 1,000 nanogram per milliliter (ng/mL)-0.01 ng/mL.

IL2 formulation as at 500 mg/mL. Diluted 1:50 in Expi293 media then 5 fold dilution series (transfered 20 mL into 80 mL Expi293). After diluting 20 mL of this to a final volume of 200 mL for the assay the concentration range is: 1000 ng/ml>200 ng/ml>40 ng/ml>8 ng/mL>1.6 ng/mL>0.32 ng/ml>0.06 ng/ml>0.012 ng/ml.

Incubated plates with dilution series of Designed Cytokines or WT IL-2 overnight at 37° C. Plated 180 mL of Quantiblue reagent per well of a 96 well flat bottom plate. Added 20 mL of culture supernatant to each well. Incubated at 37° C. for 30 min. Measured A640 on the plate reader using the Quantiblue program.

### Octet: Binding to IL2Rα

Octet setup: ProA tips, Octet Buffer (HBS-EP+0.25% BSA), use 2.6 mg/mL IL2Ra-Fc for capture; use zeba columns (0.5 mL, 7 kDa) to buffer-exchange proteins into HBS-EP (No BSA).

Prepped protein Octet dilutions in octet buffer (HBS-EP+0.25% BSA)

Hydrated tips for >10 min during experiment setup, in 200 mL buffer/well (need one column of tips).

Set-up assay plate (black 96 well plate) with protein dilutions and buffer. Use 200 mL sample/buffer per well (add octet buffer for baseline step).

| | | 3 | 4 | 5 | 6 | 7 | | |
|---|---|---|---|---|---|---|---|---|
| | 2 | Designed | Designed | Designed | Designed | Internal | | |
| | IL2Ra-Fc | Cytokine | Cytokine | Cytokine | Cytokine | WT | 11 | 12 |
| 1 | (2.6 | (nM) | (nM) | (nM) | (nM) | IL-2 | Regen- | Neutral- |
| Baseline | ug/mL) | 3000 | 3000 | 3000 | 3000 | (nM) | eration | ization |
| | | 600 | 600 | 600 | 600 | 926 | | |
| | | 120 | 120 | 120 | 120 | 185.2 | | |
| | | 24 | 24 | 24 | 24 | 37.04 | | |
| | | 4.8 | 4.8 | 4.8 | 4.8 | 7.408 | | |
| | | 0.96 | 0.96 | 0.96 | 0.96 | 1.4816 | | |
| | Buffer | 300 | 300 | 300 | 300 | 463 | | |
| | | 0 (Reference) | 0 (Reference) | 0 (Reference) | 0 (Reference) | 0 (Reference) | | |

Thawed two 20 mL aliquots of 100 mg/mL IL2Ra-Fc. Made up 1.5 mL of the loading solution in Octet buffer, so the IL2Ra-Fc concentration was at 2.6 mg/mL.

For the Designed Cytokine columns, performed a 5-fold dilution series according to the layout table above.

Prepped 300 mL of top concentration in plate, transfer 50 mL down into 200 mL buffer; at final concentration remove 50 mL so final volumes are equal across samples.

For the 300 nM sample in row G-transferred 20 mL of the top concentration sample (3 mM, row A) into 180 mL of Octet buffer. Then removed 30 mL more of 3 mM, row A sample so final volumes were equal across samples.

| | | Extinction | | Volume IL2 | |
|---|---|---|---|---|---|
| | | coefficient | Concentration | (μL) for | Volume Octet |
| Construct | A280 | (M−1cm−1) | (μM) | 3 μM | buffer (μL) |
| Designed | 0.33 | 11000.00 | 30 | 30 | 270 |
| Cytokine | | | | | |
| Designed | 0.15 | 11000.00 | 13.6363636364 | 66 | 234 |
| Cytokine | | | | | |
| Designed | 0.30 | 11000.00 | 27.2727272727 | 33 | 267 |
| Cytokine | | | | | |
| Designed | 0.48 | 15595.00 | 30.7790958641 | 29.240625 | 270.759375 |
| Cytokine | | | | | |
| Internal | 0.37 | [symbol] | 23.7255530619 | 37.9337837838 | 262.0662162162 |
| IL-2 ** | | | | | |

| Step | Time (sec) |
|---|---|
| Baseline | 60 |
| Loading | 1200 |
| Baseline_2 | 120 |
| Association | 360 |
| Dissociation | 60 |

Octet: Binding to IL2Rb/g (beta/gamma)

Octet setup info: ProA tips, Octet Buffer (HBS-EP+0.25% BSA), use 12 μg/mL IL2Rb/g-Fc for capture, Use zeba columns (0.5 mL, 7 kDa) to buffer-exchange proteins into HBS-EP (No BSA).

Prepped protein Octet dilutions in octet buffer (HBS-EP+0.25% BSA).

Hydrated tips for >10 min during experiment setup, in 200 mL buffer/well (need 5 columns of tips-new set of tips for each construct)

Set up assay plate (black 96 well plate) with protein dilutions and buffer. Use 200 mL sample/buffer per well (add octet buffer for baseline step).

Used the Evap cover for this run.

| | | 3 | 4 | 5 | 6 | 7 | | |
|---|---|---|---|---|---|---|---|---|
| | 2 | Designed | Designed | Designed | Designed | Internal | | |
| | IL2Rb/g-Fc | Cytokine | Cytokine | Cytokine | Cytokine | WT IL2 | 11 | 12 |
| 1 | (12 | (nM) | (nM) | (nM) | (nM) | (nM) | Regen- | Neutral- |
| Baseline | ug/mL) | 100 | 100 | 100 | 100 | 153.9 ** | eration | ization |
| | | 33.33333333 | 33.33333333 | 33.33333333 | 33.33333333 | 51.3 | | |
| | | 11.11111111 | 11.11111111 | 11.11111111 | 11.11111111 | 17.1 | | |
| | | 3.703703704 | 3.703703704 | 3.703703704 | 3.703703704 | 5.7 | | |
| | | 1.234567901 | 1.234567901 | 1.234567901 | 1.234567901 | 1.9 | | |
| | | 0.4115226337 | 0.4115226337 | 0.4115226337 | 0.4115226337 | 0.6333333333 | | |
| | Buffer | 100 | 100 | 100 | 100 | 153.9 | | |
| | | 0 (Reference) | 0 (Reference) | 0 (Reference) | 0 (Reference) | 0 (Reference) | | |

Thawed two 15 mL aliquots of 600 mg/mL IL2Rb/g-Fc. Made up 1.5 mL of the loading solution in Octet buffer, so the IL2Rb/g-Fc concentration was at 12 mg/mL.

For Designed Cytokine columns did a 3-fold dilution series according to the layout table above.

Prepped 1 mL of top concentration in eppendorfs and then transfer 300 mL to top well in plate. For 3-fold dilutions, transfer 100 mL down into 200 mL buffer; at final concentration remove 100 mL so final volumes are equal across samples.

| | | Extinction | | Volume IL2 | |
|---|---|---|---|---|---|
| | | coefficient | Concentration | (μL) for | Volume Octet |
| Construct | A280 | (M−1cm−1) | (μM) | 0.1 μM | buffer (μL) |
| Designed | 0.33 | 11000.00 | 30 | 3.3333333333 | 996.6666666667 |
| Cytokine | | | | | |
| Designed | 0.15 | 11000.00 | 13.6363636364 | 7.3333333333 | 992.6666666667 |
| Cytokine | | | | | |
| Designed | 0.30 | 11000.00 | 27.2727272727 | 3.6666666667 | 996.3333333333 |
| Cytokine | | | | | |
| Designed | 0.48 | 15595.00 | 30.7790958641 | 3.2489583333 | 996.7510416667 |
| Cytokine | | | | | |
| Internal | 0.37 | | 23.7255530619 | 4.2148648649 | 995.7851351351 |
| WT IL2 | | | | | |

| Step | Time (sec) |
|---|---|
| Baseline | 60 |
| Loading * | 1500 |
| Baseline_2 | 120 |
| Association | 120 |
| Dissociation | 480 |

There was an issue with the Evap cover for the last two constructs above which caused the run to fail, so we need to set up the experiment again for these.

Hydrated tips for >10 min during experiment setup, in 200 mL buffer/well (need 2 columns of tips-new set of tips for each construct)

Used buffer-exchanged Designed Cytokine proteins (in HBS-EP).

Used the same setup as for a replicate, except didn't use the Evap cover.

| | | | | | |
|---|---|---|---|---|---|
| | 2 | 3 | 4 | | |
| 1 | IL2Rb/g-Fc | WT IL-2 (nM) | Internal IL-2 (nM) | 11 | 12 |
| Baseline | (12 ug/mL) | 100 | 153.9 ** | Regeneration | Neutralization |

| | | |
|---|---|---|
| | 33.33333333 | 51.3 |
| | 11.11111111 | 17.1 |
| | 3.703703704 | 5.7 |
| | 1.234567901 | 1.9 |
| | 0.4115226337 | 0.6333333333 |
| Buffer | 100 | 153.9 |
| | 0 (Reference) | 0 (Reference) |

### Intrinsic Tryptophan Fluorescence

### Materials:

| Item | Vendor | Cat No |
|---|---|---|
| PBS pH 7.2 | Gibco | 20012-043 |
| Guanidine HCl 8M Buffered | VWR | BDH7427-1 |

### Experimental Details:

Diluted Designed Cytokine proteins to 0.2 mg/mL in Gibco PBS pH 7.2 (with one exception in grey)

| | | Protein Conc | Volume for | |
|---|---|---|---|---|
| Protein batch | mg/mL stock | (uM) | GndHCl | Volume PBS |
| Designed | 0.517 | 37.909 | 106.3829787234 | 168.6170212766 |
| Cytokine | | | | |
| Designed | 0.295 | 17.377 | 186.4406779661 | 88.5593220339 |
| Cytokine | | | | |
| Designed | 0.3208719091 | 23.636 | 171.4079619941 | 103.5920380059 |
| Cytokine | | | | |
| Designed | 0.5009110036 | 36.545 | 109.799943712 | 165.200056288 |
| Cytokine | | | | |
| Designed | 0.1861 | 13.545 | 275 | 0 |
| Cytokine | | | | |
| Designed | 0.2634730145 | 19.182 | 208.7500312105 | 66.2499687895 |
| Cytokine | | | | |
| Designed | 0.2145168971 | 15.640 | 256.3900594477 | 18.6099405523 |
| Cytokine | | | | |

*Because of the low concentration, use undiluted (this sample will be less than 0.2 mg/mL)

Added 25 μL of diluted protein to each well of a round-bottom black 96 well plate

Added 75 μL of GndHCl diluted from 8M GndHCl as shown below, with the pre- and post-mixing concentrations listed below

| | | | | Final GndHCl after | | |
|---|---|---|---|---|---|---|
| | | | GndHCl after | Diluting 75 uL of | | |
| | mL of Stock | mL of | Dilution | Stock + 25 uL | | |
| Well | GndHCl | PBS | with PBS | of Protein | | |
| 1 | 1 | 0 | 8 | 6 | M | 6M |
| 2 | 0.9 | 0.1 | 7.2 | 5.4 | M | 5.4M |
| 3 | 0.8 | 0.2 | 6.4 | 4.8 | M | 4.8M |
| 4 | 0.7 | 0.3 | 5.6 | 4.2 | M | 4.2M |
| 5 | 0.6 | 0.4 | 4.8 | 3.6 | M | 3.6M |
| 6 | 0.5 | 0.5 | 4 | 3 | M | 3M |
| 7 | 0.4 | 0.6 | 3.2 | 2.4 | M | 2.4M |
| 8 | 0.3 | 0.7 | 2.4 | 1.8 | M | 1.8M |
| 9 | 0.2 | 0.8 | 1.6 | 1.2 | M | 1.2M |
| 10 | 0 | 1 | 0 | 0 | M | 0M |

Cover, and incubate plate at 37 C for 45 min

Measure fluorescence on a BioTek Synergy H1 plate reader as follows:
37C
Excitation 280 nm
30 sec orbital mixing
Scan 310-450 nm with 5 nm step

### Example 2: Designed Cytokine Signaling

Designed Cytokines were engineered to not bind to CD25 (IL2Ra), but to activate IL2R through IL2Rb and common gamma chain receptors. This study demonstrates the ability of Designed Cytokines to express in transiently transfected Expi293 cells, to bind to CD25 (IL2Ra) by ELISA, and to activate HEK-Blue reporter lines that express [IL2Ra, IL2Rb, and the gamma subunit common to IL-2 and IL-15] OR [IL2Rb and the gamma subunit common to IL-2 and IL-15].

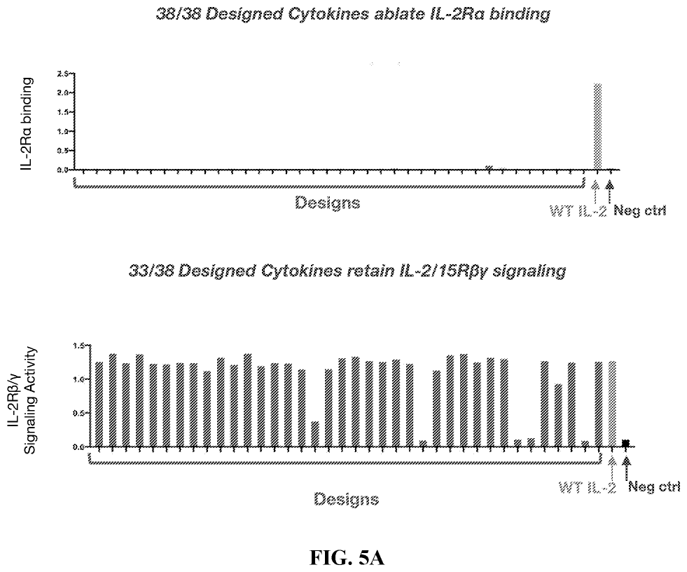

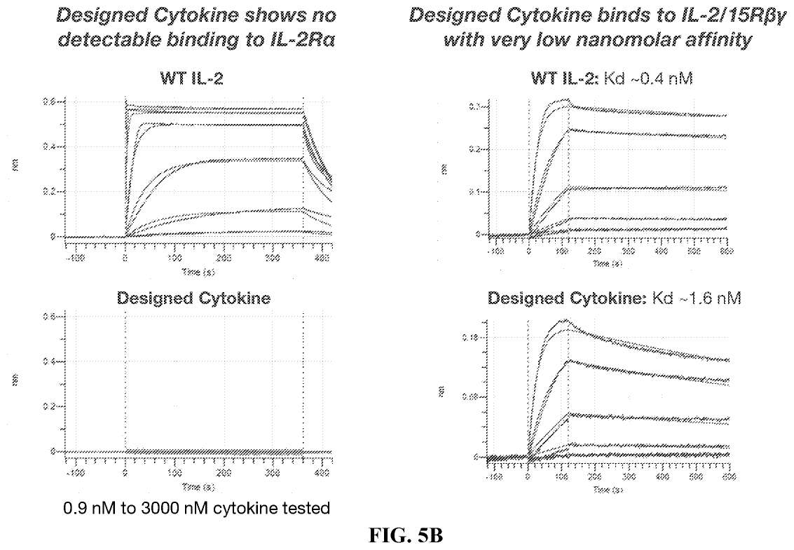

With reference to FIG. 5A and to Designed Cytokines having SEQ ID Nos 1-38, respectively, the data demonstrate that, when compared to WT IL-2, the exemplary Designed Cytokines of the disclosure do not bind or signal through the alpha subunit of the IL-2 Receptor (IL-2R).

With reference to FIG. 5A and to Designed Cytokines having SEQ ID Nos 1-38, respectively, the data demonstrate that, when compared to WT IL-2, 33 of the 38 exemplary Designed Cytokines of the disclosure retain IL-2/15Rβγ signaling.

Based on this initial screen, number of Designed Cytokines were identified that 1) express in Expi293 cells, 2) activate IL2R and CD122/132 only in HEK-Blue assays even at relatively high dilutions of the transfection supernatant, and 3) no longer bind to CD25 (despite relatively good expression). These designs were marked for further development. Among these, Designed Cytokine No. 39 and Designed Cytokine No. 40 appear to be of particular interest because these were among the highest expressing proteins by anti-STII jess. Designed Cytokine No. 39 and Designed Cytokine No. 40 showed good activation in HEK-Blue assays and they did not bind to CD25. These proteins are also closely related, so the fact that their behavior is similar confirms the structure-function relationship.

With respect to binding to IL2Rb/g, we compared Designed Cytokines to WT IL2 (both “internal” IL-2 and commercial IL2).

With reference to FIG. 5B, the data demonstrate that, the Designed Cytokines bind to IL2Rb/g with nanomolar affinities.

IL2Ra binding: No detectable binding of the Designed Cytokines to IL2Ra was observed, even up to 3 uM. WT IL2, in contrast, binds to IL2Ra and has a Kd of ˜15 nM. This experiment therefore confirms that our WT IL2 binds to IL2Ra as expected, while the Designed Cytokines show no binding.

IL2R b/g binding: The Designed Cytokines bind to IL2Rb/g with nanomolar affinity, similar to WT IL2 binding. Designed Cytokine No. 169 has a Kd of ˜1.6 nM, while WT IL2 has a Kd of ˜0.4 nM. Designed Cytokine No. 153 has a lower affinity, at 3.9 nM, which is consistent with its lower activity in functional assays.

### Example 3: Designed Cytokines have Increased Stability Compared to WT IL-2

In order to test whether the Designed Cytokines are stabilized relative to WT IL2, changes in intrinsic tryptophan fluorescence was measured at increasing concentrations of the denaturant GndHC1. Tryptophan fluorescence is sensitive to its environment, and solvent exposure causes a ‘red-shift’ in the emission spectrum of tryptophan with excitation at 280 nm.

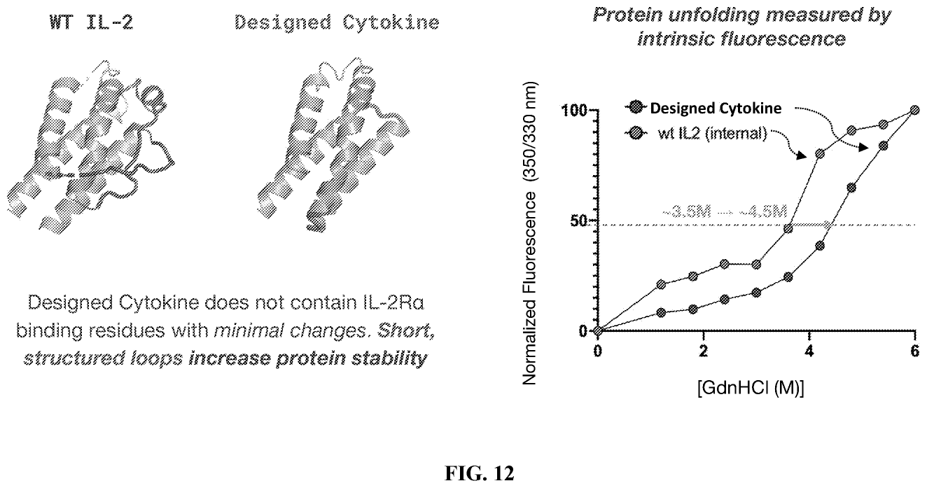

With respect to FIG. 12, the Designed Cytokines demonstrate lower intrinsic fluorescence, and therefore, greater stability upon contact with a denaturant, when compared to the intrinsic fluorescence of WT IL-12.

### OTHER EMBODIMENTS

Although the disclosure is described in some detail by way of illustration and example for purposes of clarity and understanding, it will be apparent to those skilled in the art that certain changes and modifications may be practiced. Reference to “the disclosure” or the like is intended as a reference to any of a wide variety of embodiments of, or aspects of, the disclosure, and not as limiting the disclosure to a single embodiment or aspect. As used throughout the disclosure, the terms “aspect” and “embodiment” are interchangeable. Features discussed in the context of “certain”, “some”, or “other” aspects or embodiments of the disclosure may be found in any embodiment of the disclosure, however, in these instances, the feature may be considered a preferred feature in these highlighted embodiments.

The description and examples should not be construed as limiting the scope of the disclosure to the embodiments and examples described herein, but rather as encompassing all modifications and alternatives falling within the true scope and spirit of the disclosure.

LENGTHY TABLES
The patent application contains a lengthy table section. A copy of the table is available in electronic form from the USPTO web site (https://seqdata.uspto.gov/?pageRequest=docDetail&DocID=US20250223330A1). An electronic copy of the table will also be available from the USPTO upon request and payment of the fee set forth in 37 CFR 1.19(b)(3).

We claim:

1. A designed cytokine comprising alpha helices H1, H2, H3, and H4, wherein:
from an amino terminus to a carboxy terminus,
a first loop (L1) connects H1 and H4;
a second loop (L2) connects H4 and H2;
a third loop (L3) connects H2 and H3; and
wherein the polypeptide binds to IL-2 receptor βγ (IL-2Rβγ).

2. The designed cytokine of claim 1, wherein the polypeptide does not bind to IL-2 receptor alpha (IL-2Rα).

3. The designed cytokine of claim 1 or 2, wherein the polypeptide comprises one or more of:
(a) a sequence of SEQ ID NO: 1-350 and
(b) a sequence having at least 70% identity to a sequence of (a).

4. The designed cytokine of claim 1 or 2, wherein the polypeptide comprises a sequence of SEQ ID NO: 1-38 or 150-350.

5. The designed cytokine of claim 1 or 2, wherein the polypeptide comprises the sequence of
APTSSSTKKTQLQLEHLLLDLQMILNGINNMNADPELVEFLNRWI
TFCQSIISTGSLEDLKHLQALEEELKPLEEVLNLAQSKNFHLNPR
DLISNINVLVLELK.

6. The designed cytokine of claim 1 or 2, wherein the polypeptide comprises the sequence of
APTSSSTKKTQLQLEHLLLDLQMILNGINNMNADPELVEFLNRWI
TFCQSIISTGSLEDLKHLQALEEELKPLEEVLNLAQSKNFHLRPR
DLISNINVIVLELK.

7. The designed cytokine of claim 1 or 2, wherein the polypeptide comprises the sequence of
APTSSSTKKTQLQLEHLLLDLQMILNGINNMNADPELVEFLNRWI
TFCQSIISTGSLEDLKHLQALEEELKPLEEVLNLAQSKNFHLRPR
DLISNINVIVLELK.

8. The designed cytokine of claim 1 or 2, wherein the polypeptide comprises the sequence of
APTSSSTKKTQLQLEHLLLDLQMILNGINNMNADPELVEFLNRWI
TFCQSIISTLTAGGSLSGDLKHLQNLSEELKPLEEVLNLAQSKNF
HLRPRDLISNINVIVLELK.

9. The designed cytokine of claim 1 or 2, wherein the polypeptide comprises the sequence of
APTSSSTKKTQLQLEHLLLDLQMILNGINNMNADPELVEFLNRWI
TFCQSIISTGSVDPEELAKELQKLEEELKPLEEVLNLAQSKNFHL
RPRDLISNINVIVLELK.

10. The designed cytokine of any one of claims 1-9, wherein the polypeptide is operably linked to a targeting moiety.

11. The designed cytokine of any one of claims 1-9, wherein the polypeptide comprises a targeting moiety.

12. The designed cytokine of any one of claims 1-9, wherein a fusion protein comprises the polypeptide and a targeting moiety.

13. The designed cytokine of any one of claims 10-12, wherein the targeting moiety binds to a component of a tumor microenvironment (TME).

14. The designed cytokine of any one of claims 10-13, wherein the targeting moiety binds to one or more of T-cell surface glycoprotein CD8 (also known as cluster of differentiation 8), Programmed cell death protein 1 (PD-1), Programmed death-ligand 1 (PD-L1; also known as cluster of differentiation 274 (CD274) or B7 homolog 1 (B7-H1) polypeptide), T-cell immunoreceptor with Ig and ITIM domains (TIGIT), Cytotoxic T-lymphocyte protein 4 (CTLA4), Lymphocyte activation gene 3 protein (LAG3), T-cell immunoglobulin mucin receptor 3 (TIM3).

15. The designed cytokine of any one of claims 10-13, wherein the targeting moiety binds CD8.

16. The designed cytokine of any one of claims 10-13, wherein the targeting moiety binds PD-1.

17. The designed cytokine of any one of claims 10-13, wherein the targeting moiety binds PD-L1.

18. The designed cytokine of any one of claims 10-17, wherein the targeting moiety comprises an antibody, an antibody mimetic, or a functional fragment thereof.

19. The designed cytokine of claim 18, wherein the targeting moiety comprises one or more of a monoclonal antibody, an antigen-binding fraction (Fab), a single-chain variable fraction (scFv), a domain antibody, one or more of a heavy chain (VH) and a light chain (VL) domain of an immunoglobulin (Ig) polypeptide or gene encoding the same, a heavy-chain antibody (a VH or a VHH), a camelid or camelid-like structured antibody, and a nanobody.

20. The designed cytokine of claim 18, wherein the targeting moiety comprises a scFv.

21. The designed cytokine of claim 18, wherein the targeting moiety comprises a VHH.

22. The designed cytokine of any one of claims 1-21, wherein the polypeptide is operably linked to a tether.

23. The designed cytokine of any one of claims 1-21, wherein the polypeptide comprises a tether.

24. The designed cytokine of any one of claims 1-21, wherein a fusion protein comprises the polypeptide and a tether.

25. The designed cytokine of any one of claims 22-24, wherein the tether comprises one or more of a nucleic acid sequence, an amino acid sequence, a small molecule.

26. The designed cytokine of any one of claims 22-25, wherein the tether comprises a sequence isolated or derived from a transmembrane sequence.

27. The designed cytokine of any one of claims 22-26, wherein the tether comprises the sequence PLFIPVAVMVTAFSGLAFIIWLARRLKKGKK.

28. The designed cytokine of any one of claims 1-27, wherein the polypeptide is operably linked to a second cytokine or a second Designed Cytokine.

29. The designed cytokine of any one of claims 1-27, wherein the polypeptide comprises a a second cytokine or a second Designed Cytokine.

30. The designed cytokine of any one of claims 1-27, wherein a fusion protein comprises the polypeptide and a second cytokine or a second Designed Cytokine.

31. The designed cytokine of any one of claims 28-30, wherein the second cytokine comprises a sequence isolated or derived from one or more of an IL-2 polypeptide, an IL-12 polypeptide, an IL-15 polypeptide, an IL-18 polypeptide, an IL-21 polypeptide, an IL-23 polypeptide, an interferon alpha polypeptide, an interferon beta polypeptide, an interferon gamma polypeptide, and an interferon omega polypeptide.

32. The designed cytokine of claim 31, wherein the polypeptide comprises a first targeting moiety and the second cytokine comprises a second targeting moiety.

33. The designed cytokine of claim 32, wherein the first targeting moiety and the second targeting moiety are identical.

34. The designed cytokine of claim 32, wherein the first targeting moiety and the second targeting moiety are not identical.

35. The designed cytokine of any one of claims 28-30, wherein the second Designed Cytokine comprises a sequence of any one or more of SEQ ID NO: 1-38 or 150-350.

36. The designed cytokine of claim 35, wherein the polypeptide comprises a first targeting moiety and the second Designed Cytokine comprises a second targeting moiety.

37. The designed cytokine of claim 36, wherein the first targeting moiety and the second targeting moiety are identical.

38. The designed cytokine of claim 36, wherein the first targeting moiety and the second targeting moiety are not identical.

39. The designed cytokine of claim 31, wherein the polypeptide comprises a first tether and the second cytokine comprises a second tether.

40. The designed cytokine of claim 39, wherein the first tether and the second tether are identical.

41. The designed cytokine of claim 39, wherein the first tether and the second tether are not identical.

42. The designed cytokine of claim 35, wherein the polypeptide comprises a first tether and the second Designed Cytokine comprises a second tether.

43. The designed cytokine of claim 36, wherein the first tether and the second tether are identical.

44. The designed cytokine of claim 36, wherein the first tether and the second tether are not identical.

45. A nucleic acid encoding the designed cytokine of any one of claims 1-44 or a fusion protein comprising the designed cytokine of any one of claims 1-44.

46. The nucleic acid of claim 45, further comprising a regulatory element capable of driving expression of the designed cytokine.

47. The nucleic acid of claim 46, wherein the regulatory element comprises a promoter.

48. The nucleic acid of claim 47, wherein the promoter comprises a minimal promoter.

49. The nucleic acid of claim 48, wherein the minimal promoter comprises a sequence isolated or derived from one or more of minimal promoter-1 (“minP1”), YB-TATA and human beta globin.

50. The nucleic acid of claim 49, wherein the minP1 comprises a sequence of
AGAGGGTATATAAAAGCTCGACTTCCAG.

51. The nucleic acid of claim 49, wherein the minimal promoter comprises the sequence of
TAGAGGGTATATAATGGGGGCCACTAGTCTACTACCAGAAAGCTT
GGTACCGAGCTCGGATCCAGCCACC.

52. The nucleic acid of claim 49, wherein the minimal promoter comprises the sequence of
CTAGAGGGTATATAATGGGGGCCACTAGTCTACTACCAGAAAGCT
TGGTACCGAGCTCGGATCCAGCCACC.

53. The nucleic acid of any one of claims 47-52, wherein the promoter is inducible.

54. The nucleic acid of any one of claims 47-53, wherein the regulatory element comprises a response element.

55. The nucleic acid of claim 54, wherein the regulatory element comprises a noncoding or an untranslated sequence isolated or derived from one or more of NFAT, NFkB, REL, RELA, IRF2, GATA3 and ATF3.

56. The nucleic acid of claim 54, wherein the regulatory element comprises a noncoding or an untranslated sequence isolated or derived from a GATA3 gene and, optionally, wherein the GATA3 sequence comprises GTTATCTCTCACGAGATCT.

57. The nucleic acid of claim 54, wherein the regulatory element comprises a noncoding or an untranslated sequence isolated or derived from RELA and, optionally, wherein the RELA sequence comprises GGGGATTTCCA.

58. The nucleic acid of any one of claims 47-57, wherein the response element comprises a repeated sequence.

59. A vector comprising a nucleic acid of any one of claims 45-58.

60. The vector of claim 59, wherein the vector comprises an expression vector.

61. The vector of claim 59, wherein the vector comprises a delivery vector.

62. The vector of any one of claims 59-61, wherein the vector further comprises a sequence encoding an exogenous receptor.

63. The vector of claim 62, wherein the exogenous receptor comprises an antigen binding moiety.

64. The vector of claim 63, wherein the exogenous receptor comprises a T Cell Receptor (TCR).

65. The vector of claim 63, wherein the exogenous receptor comprises a chimeric antigen receptor (CAR).

66. The vector of any one of claims 62-65, wherein the antigen is expressed on or secreted within one or more of a tumor cell, a cancer cell, a component of a TME, and a TME.

67. A cell comprising a designed cytokine of any one of claims 1-44.

68. A cell comprising a nucleic acid of any one of claims 45-58.

69. A cell comprising a vector of any one of claims 59-66.

70. The cell of any one of claims 67-69, wherein the cell is a mammalian cell.

71. The cell of claim 70, wherein the cell is a human cell.

72. The cell of any one of claims 67-71, wherein the cell is a primary cell.

73. The cell of any one of claims 67-71, wherein the cell is a cultured cell.

74. The cell of claim 73, wherein the cultured cell is an immortalized cell.

75. The cell of any one of claims 67-74, wherein the cell is ex vivo or in vitro.

76. The cell of any one of claims 67-71, wherein the cell is in vivo.

77. The cell of any one of claims 67-76, wherein the cell is an immune cell.

78. The cell of claim 77, wherein the cell is a stem cell or a precursor cell capable of producing the immune cell.

79. The cell of claim 78, wherein the stem cell is a hematopoietic stem cells (HSC), an induced pluripotent stem cell (iPSC) or a dedifferentiated immune cell.

80. The cell of any one of claims 67-79, wherein the immune cell is a T lymphocyte (T cell), a B lymphocyte (B cell), a macrophage or a natural killer (NK) cell.

81. The cell of any one of claims 67-79, wherein the immune cell is a T cell.

82. The cell of claim 81, wherein the T cell is an alpha beta T cell.

83. The cell of claim 81, wherein the T cell is a gamma delta T cell.

84. The cell of any one of claims 67-79, wherein the immune cell is a NK cell.

85. A composition comprising a designed cytokine of any one of claims 1-44.

86. A composition comprising a nucleic acid of any one of claims 45-58.

87. A composition comprising a vector of any one of claims 59-66.

88. A composition comprising a cell of any one of claims 67-84.

89. A pharmaceutical composition comprising one or more of (1) a designed cytokine of any one of claims 1-44, a nucleic acid of any one of claims 45-58, a vector of any one of claims 59-66, and a cell of any one of claims 67-84 and (2) a pharmaceutically acceptable carrier.

90. The use of a designed cytokine of any one of claims 1-44, a nucleic acid of any one of claims 45-58, a vector of any one of claims 59-66, a cell of any one of claims 67-84 or a pharmaceutical composition of claim 71 in the manufacture of a medicament for the treatment of a disease or condition.

91. A designed cytokine of any one of claims 1-44, a nucleic acid of any one of claims 45-58, a vector of any one of claims 59-66, a cell of any one of claims 67-84 or a pharmaceutical composition of claim 89 for use in the treatment of a disease or condition.

92. The use of claim 90 or 91, wherein the disease or disorder comprises a cancer or a subtype thereof.

93. The use of claim 92, wherein the cancer or the subtype thereof comprises a liquid cancer.

94. The use of claim 92, wherein the cancer or the subtype thereof comprises a hematological cancer.

95. The use of claim 92, wherein the cancer or the subtype thereof comprises a solid cancer.

96. A method of treating a disease or disorder comprising administering to a subject an effective amount of a designed cytokine of any one of claims 1-44, a nucleic acid of any one of claims 45-58, a vector of any one of claims 59-66, a cell of any one of claims 67-84 or a pharmaceutical composition of claim 89, wherein a severity of a sign or symptom of the disease or disorder is decreased, thereby treating the disease or disorder.

97. A method of preventing a disease or a disorder, comprising administering to a subject an effective amount of a designed cytokine of any one of claims 1-44, a nucleic acid of any one of claims 45-58, a vector of any one of claims 59-66, a cell of any one of claims 67-84 or a pharmaceutical composition of claim 89, wherein an onset or a relapse of a sign or symptom of the disease or disorder is delayed or inhibited, thereby preventing the disease or disorder.

98. The method of claim 96 or 97, wherein the disease or disorder comprises a cancer or a subtype thereof.

99. The method of claim 98, wherein the cancer or the subtype thereof comprises a liquid cancer.

100. The method of claim 98, wherein the cancer or the subtype thereof comprises a hematological cancer.

101. The method of claim 98, wherein the cancer or the subtype thereof comprises a solid cancer.
